(19) United States
(12) Patent Application Publication
(10) Pub. No.: US 2022/0332789 A1
(43) Pub. Date: Oct. 20, 2022

(54) ANTI-IgE CONSTRUCT

(71) Applicants: Combikine Biotechnology Limited, Haywards Heath (GB); King's College London, Greater London (GB)

(72) Inventors: Charles Owen, Haywards Heath (GB); Andrew Beavil, Greater London (GB); Rebecca Beavil, GreaterLondon (GB)

(73) Assignees: Combikine Biotechnology Limited, Haywards Heath (GB); King's College London, Greater London (GB)

(21) Appl. No.: 17/616,376

(22) PCT Filed: Jun. 5, 2020
(86) PCT No.: PCT/GB2020/051377

§ 371 Date: Dec. 3, 2021

(30) Foreign Application Priority Data
Jun. 6, 2019 (GB) 1908108.2

(51) Int. Cl. C07K 14/705; C07K 16/42

(52) U.S. Cl. CPC C07K 14/7056; C07K 16/4291; C07K 2319/30; C07K 2317/52; C07K 2317/92; C07K 2317/76; C07K 2317/77

(57) ABSTRACT

The present invention provides a protein construct comprising: a) at least two monomers each of which comprises a C-type lectin domain of CD23, wherein each monomer can bind to IgE; and b) an entity which can bind to the neonatal Fc receptor (FcRn); wherein said protein construct comprises a linker, and wherein said linker is used to link said monomer comprising a C-type lectin domain of CD23 to said entity which can bind to FcRn. Therapeutic uses of the constructs, for example in anti-IgE therapy or for use in the treatment or prevention of an IgE related disease or condition are also provided.

The present invention relates to a novel type of protein construct which has the ability to bind to IgE and also to bind to FcRn. Such constructs or molecules will have therapeutic uses in anti-IgE therapies and potentially provide significant benefits over existing therapies.

The therapeutic antibody field has been transformative to the life of patients, many previously without effective clinical options. Monoclonal antibody therapeutics have a number of significant advantages compared with traditional drugs, including outstanding target specificity and relatively long half lives in patients. However, at present there remain a large number of diseases, potential targets and therapeutic opportunities that neither monoclonal antibodies, nor novel formats are capable of addressing, for example due to high target turnover or targets expressed at high levels. The next generation of antibody-based therapeutics must overcome a number of these current constraints, do so at reasonable cost and be acceptable to patients.

Target-mediated drug disposition (TMDD) is a well known property of antibody therapeutics in patients. In a healthy subject, the pharmacokinetic behaviour of a therapeutic monoclonal antibody is much the same as a normal IgG, with a half-life of 21-23 days (Lowe et al., Basic Clin Pharm Tox 106; 195-209, 2009). In the presence of a soluble ligand, the half-life of a therapeutic monoclonal antibody shifts towards the half-life of the target ligand. Hence, for soluble target ligands with a short half-life, the antibody-ligand complex is cleared from the circulation relatively quickly (Lowe et al., supra). This phenomenon creates a problem for monoclonal antibodies. For target ligands with a rapid turnover (e.g. chemokines, some immunoglobulins like IgE, and cytokines), and also for targets expressed at high levels, the amount of monoclonal antibody available to sequester ligand rapidly becomes limiting and it means that the monoclonal antibodies have to be administered at correspondingly high quantities or at increased frequency in order to maintain appropriate antibody to ligand ratios to achieve the desired effect.

For antibody therapeutics, this is particularly problematic, as they are usually administered by either subcutaneous or intravenous injection. For patients, the volume of drug needing to be administered can become an unacceptable burden and has a significant effect on patient compliance and persistence on therapy. In this regard, the inherent solubility of most commercial preparations of monoclonal antibodies is between 100-150 mg/mL, whilst the maximum tolerable injection volume is about 1 mL per injection site for subcutaneous administration. These properties set a natural limit for the dose of administered drug for monoclonal antibodies without resorting to intravenous infusions.

However, for target ligands with even moderately high levels or moderately rapid turnover in patients of normal body weight, this can mean multiple high volume injections (e.g. multiple 1 ml injections) at frequent intervals (e.g. every 2 to 4 weeks) to sequester the target ligand to the extent required for efficacy. This becomes highly burdensome for the patient and the health care system.

Thus, the recent trend for therapeutic antibodies has been to develop very high affinity antibodies to achieve neutralisation at antibody:ligand ratios approaching 1:1, as a means to achieve superior efficacy and lower doses for patients. However, often such high affinity antibodies suffer from other problems such as loss of solubility or a reduction in other physiochemical properties.

Thus, alternative solutions to the problems encountered for target ligands with a rapid turnover and also for targets expressed at high levels are desired. In addition, for all targets, alternative means by which dose volumes or frequency of administration could be reduced would represent a welcome advance in the art and would potentially be transformative for therapy.

As mentioned above, IgE is an example of one of these difficult targets. IgE plays a key role in allergy, which, in general terms, occurs when the body responds to an otherwise innocuous substance. Allergic diseases such as asthma, rhinitis, eczema and food allergy are becoming more prevalent worldwide and pose a significant burden to the healthcare system. IgE plays a central role in allergy and interacts with two receptors; the so-called “high” affinity receptor, FcεRI and the so-called “low” affinity receptor, CD23 (also referred to as FcεRII).

The high affinity IgE receptor, FcεRI, is found on cell types such as mast cells and basophils (Sutton and Gould, 2008, Nat. Rev. Immunol., 8, 205; Sutton and Davies, 2015, Immunol. Rev. 268:222-235). Cross-linking of FcεRI-bound IgE by allergen results in mast cell and basophil degranulation, and the release of inflammatory mediators such as histamine, cytokines/chemokines and proteases.

The low affinity IgE receptor, CD23 (FcεRII), which binds to free IgE with low μM affinity, is found on a variety of cell types including B cells, activated macrophages, eosinophils, monocytes, dendritic cells, platelets and endothelial cells. CD23 has a C-type lectin “head” domain connected to the cell membrane by a “stalk” domain, followed by a short intracellular/cytoplasmic “tail” domain at the N-terminus. The membrane-bound form of CD23 (also referred to as mCD23) is a type II transmembrane glycoprotein of approximately 45 kDa and is usually found as a trimer in which three of the head domains are connected to the membrane by three individual stalk domains, which together form a trimeric a helical coiled-coil stalk. CD23 is believed to have multiple biological roles, including a role in transcytosis of allergens across epithelia by the formation of IgE immune complexes. CD23 also plays a role in antigen presentation as well as regulation of the IgE response via CD21 binding. Human CD23 has two isoforms: CD23a (endocytosis) and CD23b (phagocytosis), which differ in their cytoplasmic domains and hence signalling properties.

Soluble CD23 (also referred to as sCD23) is formed by cleavage of mCD23 from the cell surface. sCD23 is a freely soluble protein which can still participate in biological processes and functions, for example ligand binding, in particular binding to IgE. A range of freely soluble CD23 (sCD23) proteins are found naturally, e.g. proteins of 37 kDa, 33 kDa, 25 kDa and 16 kDa, all of which bind IgE and have cytokine-like activities. A protease which can be responsible for CD23 release from cells is the metalloprotease ADAM10, which cleaves at the C-terminal side of Alanine 80 (A80) in human CD23 to generate the 37 kDa sCD23 molecule, or cleaves at the C-terminal side of Arginine 101 (R101) to generate the 33 kDa species (Lemieux et al., J. Biol. Chem., 2007, 282:14836-14844). A further naturally occurring sCD23 fragment is derCD23, which is produced by action of the der p1 protease found in the faeces of the house dust mite Dermatophagoides pterronysinus. The der p1 protease cleaves between Serine 155 (S155) and Serine 156 (S156) and between Glutamic acid 298 (E298) and Serine 299 (S299) in human CD23 to yield the 16 kDa derCD23 fragment, which is monomeric rather than trimeric (Schultz et al., 1997, Eur. J. Immunol. 27:584-588).

FcεRI and CD23 (FcεRII) bind to IgE at distinct sites (Dhaliwal et al., 2017, Sci. Rep. 7, 45533). The binding is allosterically regulated such that IgE cannot bind both types of receptors simultaneously (Dhaliwal et al., 2017, supra). The constant region 3 of IgE (Cε3) is key to the binding of both FcεRI and CD23 to IgE. In this regard, FcεRI binds to IgE when the Cε3 domains have adopted a so-called “open” conformation, whereas CD23 binds to IgE when the Cε3 domains have adopted a so-called “closed” conformation. Importantly, CD23 binding to IgE can lock IgE in the closed conformation thus preventing binding of IgE to FcεRI. In general, two separate molecules of CD23 (e.g. two CD23 monomers or two CD23 trimers) bind to the closed conformation of IgE; one CD23 molecule binding to the Cε3 domain in each of the two chains of the IgE Fc dimer.

The current benchmark in terms of approved drugs for anti-IgE therapy is Omalizumab (Xolair®, Novartis), which is an anti-IgE monoclonal IgG1 antibody (Holgate et al., 2005, J. Allergy Clin. Immunol. 115, 459-465). Omalizumab acts by binding to free IgE and preventing it from binding to FcεRI (i.e. by competitive inhibition), thereby preventing mast cell degranulation and basophil activation. Omalizumab has been approved for the treatment of severe persistent allergic asthma and chronic idiopathic urticaria. It is administered according to a dosing table based on body weight and baseline level of IgE in blood. However, its moderate binding affinity requires significant drug excess to achieve effective suppression of IgE, leading to complex dosing and sub-optimal clinical outcomes. Ligelizumab is another anti-IgE monoclonal antibody which is in late stage development and has been developed for its high affinity binding to IgE. However, the mechanism of action is otherwise the same as Omalizumab and the high affinity binding has not addressed all of the issues. For example, as set out above, the high affinity binding likely leads to rapid consumption of the drug via target mediated drug disposition (TMDD), and therefore a need for higher and more frequent doses than might be anticipated (Arm et al., 2014, Clinical and Experimental Allergy, 44:1371-1385). The mechanisms responsible for the rapid clearance of IgE itself are poorly understood. It is likely that IgE receptor uptake of IgE plays a role in its consumption, as well as endocytic uptake and degradation through the lysosomal degradation pathway, since IgE does not possess FcRn binding (Lawrence et al., J. Allergy Clin. Immunol., 2017, 139(2):422-428).

FcRn is a type 1 membrane glycoprotein which is largely expressed within acidic intracellular compartments such as endosomes (Sand et al., 2015, Frontiers in Immunol. 5, article 682; Grevys et al., 2018, Nat. Commun. 9:621-635). One of the known roles for FcRn is in recycling of certain molecules such as IgG or albumin back to the serum following endocytosis. For example, FcRn interacts with the Fc region of IgG at the CH2-CH3 domain interface with 2:1 stoichiometry (i.e. one molecule of IgG-Fc binds to two molecules of FcRn). Recycling is facilitated by pH-dependent binding of IgG-Fc to FcRn. In this regard, IgG-Fc binds FcRn with high affinity at pH 6.0/6.5, but not at pH 7.4. In this way, FcRn binds to IgG in the acidified endosomes (via the IgG-Fc region), but IgG then dissociates from FcRn at physiological/neutral pH, e.g. when the recycling endosomes containing FcRn-IgG complexes fuse with the cell membrane thereby releasing IgG back into the serum (Roopenian and Akilesh, 2007, Nat. Rev. Immuno. 7:715-725; Sand et al., 2015, supra; Grevys et al., 2018, supra).

In this way, IgG sub-types of therapeutic antibodies such as Omalizumab are to a certain extent recycled back into the circulation. However, as discussed above, there are certain disadvantages with current anti-IgE therapies which need to be solved. For example, although Omalizumab therapy is effective, antibody therapies are expensive and high doses of Omalizumab are needed to target and block all the free IgE molecules present. Although this problem can potentially be alleviated somewhat by for example generating antibodies with higher affinity, such as Ligelizumab, the amount of free IgE in allergy still represents a significant problem. In addition, as discussed above, much of the therapeutic anti-IgE antibody is cleared rapidly, by virtue of TMDD. Thus, repeated doses of drug are required as it is cleared from the body. Although some of the drug is recycled via FcRn as described above, some of the recycled drug may still be bound to the IgE target resulting in elevated levels of complexes of IgE-anti-IgE in serum (Lawrence et al., 2017, supra), so this does not alleviate the problem with repeated doses of drug being required to maintain levels of free drug.

Thus, there is a clear need for alternative and improved anti-IgE therapies. Advantageously, the present invention provides the means for such an alternative therapy which, in addition, has clear advantages over prior therapies. In this regard, the protein constructs of the present invention combine FcRn mediated recycling of the protein construct (biologic) with the removal of IgE within the cell by degradation. The recycling of the construct (biologic) means that the biologic is returned to the serum in order to bind to further molecules of IgE target. Importantly however, IgE is additionally removed or “mopped up” from the body by way of it being degraded inside cells rather than remaining in the body to cause further disease. The constructs of the present invention thus provide a novel route for destroying IgE, for example in the tissues and cells that bear FcRn in addition to the endogenous IgE clearance mechanisms, with the additional advantage that the biologic constructs are recycled rather than destroyed alongside the IgE.

The recycling element is achieved by virtue of the protein constructs of the invention comprising an entity which can bind to FcRn, such as an IgG Fc region. The degradation of IgE is surprisingly achieved by virtue of the protein constructs of the present invention comprising soluble CD23 (or a fragment or variant thereof), in particular comprising at least one molecule of a C-type lectin head domain (CTLD) of soluble CD23 (or a fragment or variant thereof), which can bind to IgE under physiological conditions observed in the tissues or serum, such as high calcium levels/calcium ion concentrations (of ˜2 mM) or neutral pH (e.g. ˜pH 7.4), but which show significantly reduced binding to IgE under endosomal conditions, such as low calcium (3-30 μM) or a reduced (or low) pH of around 5.0 to 6.5. This means that the protein constructs can bind to IgE in serum (or in the tissues) but then, when the IgE containing complexes are internalised or pinocytosed (e.g. micro-pinocytosed) or endocytosed into the cells and reach the early endosomal compartment where calcium levels are low and the environment is more acidic, the IgE is released and then enters the lysosomal pathway where it can be degraded. On the other hand, the FcRn binding part of the construct can bind to FcRn under the endosomal conditions such as reduced (low) pH or reduced (low) calcium to allow recycling of the unloaded biologic back into the serum (or tissues) to bind to more IgE target.

As will be explained in more detail elsewhere herein, the use of soluble CD23 (or a fragment or variant thereof), in particular comprising at least one molecule of a C-type lectin head domain of soluble CD23 (or a fragment or variant thereof) also advantageously provides a different mechanism of action from anti-IgE therapeutics such as Omalizumab and Ligelizumab, as it offers allosteric inhibition of IgE binding to FcεRI as opposed to competitive inhibition which blocks IgE binding to both FcεRI and FcεRII.

It can be seen that the molecules of the present invention thus offer several advantages over prior anti-IgE therapies. Firstly, the anti-IgE construct (biologic) of the present invention has significantly increased useful longevity in the body. Put another way it has a significantly increased half-life by virtue of the recycling of unloaded biologic back to the serum. This has a number of advantages, including the possibility to administer lower doses of drug and/or less frequent administrations of drug, with the corresponding positive and convenient experience for patients, such as smaller injection volumes and less frequent visits to health professionals. In this regard, the release of IgE and recycling of the drug can be used to overcome issues with TMDD and to maintain high serum drug levels.

Secondly, the protein constructs of the present invention, unlike other anti-IgE therapies, actually enable the removal of significant amounts of IgE from the body, for example by facilitating degradation in the lysosomes. It is believed that this will allow the possibility of complete elimination of IgE at acceptable doses and will also allow the treatment of subjects with IgE levels that are too high for existing treatments such as Omalizumab and Ligelizumab (in other words there should be no theoretical upper limit of IgE levels in potential patients). The efficient blockade by the biologic of the present invention of IgE binding to both IgE receptors, CD23 and FcεRI, should allow for its broad use in the treatment of IgE mediated diseases such as chronic spontaneous urticaria, asthma, allergic rhinitis, etc.

Thirdly, the constructs of the present invention are generally significantly smaller than whole antibodies such as Omalizumab and Ligelizumab, which gives rise to advantages in terms of drug product properties and tissue distribution. Fourthly, there are potential safety benefits as the constructs of the present invention have been shown not to induce cross-linking of IgE sensitized effector cells. In contrast, Omalizumab and Ligelizumab have high levels of circulating IgE-anti-IgE complexes which increase the risk of adverse events. These should not exist for the biologic of the present invention as the target can be rapidly destroyed. In addition, as will be described in more detail elsewhere herein, the biologic of the present invention allows for allosteric inhibition and the blocking of IgE binding to both receptors independently of affinity. By contrast, Ligelizumab and Omalizumab, are both pharmacological competitive inhibitors of IgE binding to its receptors, such that there is a requirement to maintain anti-IgE concentration above a minimum threshold, in excess of serum free IgE concentration, in order to maintain inhibition during patient treatment.

Thus, at its broadest the present invention provides a protein construct comprising:
a) at least one molecule of soluble CD23 or a fragment or variant thereof, in particular comprising at least one molecule of a C-type lectin domain (CTLD) of soluble CD23 (or a fragment or variant thereof), which can bind to IgE; and
b) an entity which can bind to the neonatal Fc receptor (FcRn).

Thus, in some embodiments, constructs may contain a single (one) molecule of soluble CD23 or a fragment or variant thereof, in particular comprising at least one molecule of a C-type lectin domain (CTLD) of soluble CD23 (or a fragment or variant thereof), which can bind to IgE.

In some embodiments it is preferred that at least two molecules of soluble CD23 (or a fragment or variant thereof), in particular comprising at least one molecule of a CTLD of soluble CD23 (or a fragment or variant thereof) are present.

In some embodiments it is preferred that the molecules of soluble CD23 (or a fragment or variant thereof), in particular comprising at least one molecule or at least two molecules of a CTLD of soluble CD23 (or a fragment or variant thereof) are monomers.

Viewed alternatively, in some embodiments it is preferred that the molecules of soluble CD23 (or a fragment or variant thereof), in particular comprising at least one molecule of a CTLD of soluble CD23 (or a fragment or variant thereof) do not have the ability to homodimerise or homotrimerise or form homooligomers.

Thus, in preferred embodiments the present invention provides a protein construct comprising:
a) at least two monomers each of which comprises a C-type lectin domain (CTLD) of CD23 or a fragment or variant thereof, wherein each monomer can bind to IgE; and
b) an entity which can bind to the neonatal Fc receptor (FcRn).

The presence of linkers/linker molecules, in particular between part a) and part b) of the constructs of the invention, is believed to confer significant advantages in terms of functionality of the constructs. Thus, in other preferred embodiments, such linkers, e.g. as described in more detail elsewhere herein, are present in the constructs.

In a preferred embodiment the present invention provides a protein construct comprising:
a) at least two monomers each of which comprises a C-type lectin domain (CTLD) of CD23 or a fragment or variant thereof, wherein each monomer can bind to IgE; and
b) an entity which can bind to the neonatal Fc receptor (FcRn); wherein said protein construct comprises a linker, and wherein said linker is used to link said monomer comprising a C-type lectin domain of CD23 to said entity which can bind to FcRn.

CD23 is found in two isoforms in humans, CD23 isoform a (CD23a, SEQ ID NO:1, NCBI NP_001207429.1, which is 321 amino acids in length) and CD23 isoform b (CD23b, SEQ ID NO:2, NCBI NP_001193948.2, which is 320 amino acids in length).

| (SEQ ID NO: 1) | |
|---|---|
| 1 | meegqyseie elprrrccrr gtqivllglv taalwagllt llllwhwdtt qslkqleera |
| 61 | arnvsqvskn leshhgdqma qksqstqisq eleelraeqq rlksqdlels wnlnglqadl |
| 121 | ssfksqelne rneasdller lreevtklrm elqvssgfvc ntcpekwinf qrkcyyfgkg |
| 181 | tkqwvharya cddmegqlvs ihspeeqdfl tkhashtgsw iglrnldlkg efiwvdgshv |
| 241 | dysnwapgep tsrsqgedcv mmrgsgrwnd afcdrklgaw vcdrlatctp pasegsaesm |
| 301 | gpdsrpdpdg rlptpsaplh s |
| (SEQ ID NO: 2) | |
| 1 | mnppsqeiee lprrrccrrg tqivllglvt aalwaglltl lllwhwdttq slkqleeraa |
| 61 | rnvsqvsknl eshhgdqmaq ksqstqisqe leelraeqqr lksqdlelsw nlnglqadls |
| 121 | sfksqelner neasdllerl reevtklrme lqvssgfvcn tcpekwinfq rkcyyfgkgt |
| 181 | kqwvharyac ddmegqlvsi hspeeqdflt khashtgswi glrnldlkge fiwvdgshvd |
| 241 | ysnwapgept srsqgedcvm mrgsgrwnda fcdrklgawv cdrlatctpp asegsaesmg |
| 301 | pdsrpdpdgr lptpsaplhs |

These two isoforms are identical in sequence, apart from the residues shown in italics at the N-terminus which are part of the cytoplasmic domain. The sequence of the N-terminal cytoplasmic domain can also vary between species. For example, the sequences are different in murine and human CD23 molecules. However, species differences in CD23 can be found throughout the molecules, not just the cytoplasmic region, although overall there are significant homologies between different species, for example in the CTLD.

From N-terminus to C-terminus, CD23 is made up of a cytoplasmic tail region, a transmembrane domain, a neck region, a stalk region and the head region (which includes a lectin head domain/C-type lectin head domain or C-type lectin domain (CTLD) and C-terminal tail that contains a CD21 binding site). There are a number of structural features in human CD23. For example, CD23 contains an MHC class II binding domain, an integrin binding site, a CD21 binding site and an IgE binding domain. The integrin binding site, CD21 binding site and IgE binding domain are all located in the head region. In addition, CD23 contains target sites for proteases, which are shown underlined in SEQ ID NO:1 above. The sequences are located at A80, R101, S155 and E298. A80 and R101 in native CD23 are believed to be cleaved by proteases in the ADAM family, in particular ADAM 10. S155 and E298 in native CD23 are believed to be the sites of cleavage for the der p1 protease found in dust mites. Generally the proteases cleave to the C-terminal side of the indicated residues.

Although these native protease sites are preferred cleavage sites for the formation of soluble CD23 (and soluble CD23 molecules produced or formed by cleavage at these sites or CD23 molecules corresponding to such soluble CD23 molecules are also preferred for use in the invention), any soluble CD23 molecule (or CTLD molecule) as defined herein can be used in or to produce the protein constructs of the present invention.

The term “soluble” as used herein with reference to CD23 molecules refers to CD23 molecules or forms of CD23 molecules that are not bound to or otherwise associated with the membrane of a cell or which can circulate freely or be freely soluble. Such soluble CD23 molecules thus include all or part of the extracellular domains of CD23 molecules, or fragments or variants thereof.

CD23 can be cleaved from cell surfaces to yield a range of soluble CD23 (sCD23) proteins/molecules and any of these can be used in the present invention. Equally however soluble CD23 molecules for use in the constructs of the present invention can be engineered or produced recombinantly. For example, an exemplary soluble CD23 for use in the constructs of the present invention could comprise or correspond to the whole extracellular domain of CD23, or a fragment or variant thereof. For example, for human CD23 an exemplary soluble CD23 molecule comprises or corresponds to the sequence D48 to S321 of SEQ ID NO:1 (SEQ ID NO:3), or a fragment or variant thereof (or a corresponding or equivalent sequence in other forms of CD23, e.g. other species of CD23). Such sCD23 molecules might contain the head plus the stalk domain of CD23, or the head plus stalk plus neck domain of CD23.

(SEQ ID NO: 3)
dtt qslkqleera
arnvsqvskn leshhgdqma qksqstqisq eleelraeqq rlksqdlels wnlnglqadl
ssfksqelne rneasdller lreevtklrm elqvssgfvc ntcpekwinf qrkcyyfgkg
tkqwvharya cddmegqlvs ihspeeqdfl tkhashtgsw iglrnldlkg efiwvdgshv
dysnwapgep tsrsqgedcv mmrgsgrwnd afcdrklgaw vcdrlatctp pasegsaesm
gpdsrpdpdg rlptpsaplh s

Another exemplary soluble CD23 for use in the constructs of the present invention could comprise or correspond to the A80 fragment of CD23, which is obtained or obtainable by cleavage (for example by ADAM10 protease cleavage) at A80 of SEQ ID NO:1, or a fragment or variant thereof. For example, for human CD23 an exemplary soluble CD23 molecule comprises or corresponds to the sequence Q81 to S321 of SEQ ID NO:1 (SEQ ID NO:4), or a fragment or variant thereof (or a corresponding or equivalent sequence in other forms of CD23, e.g. other species of CD23).

(SEQ ID NO: 4)
qksqstqisq eleelraeqq rlksqdlels wnlnglqadl
ssfksqelne rneasdller lreevtklrm elqvssgfvc ntcpekwinf qrkcyyfgkg
tkqwvharya cddmegqlvs ihspeeqdfl tkhashtgsw iglrnldlkg efiwvdgshv
dysnwapgep tsrsqgedcv mmrgsgrwnd afcdrklgaw vcdrlatctp pasegsaesm
gpdsrpdpdg rlptpsaplh s

Another exemplary soluble CD23 for use in the constructs of the present invention could comprise or correspond to the R101 fragment of CD23, which is obtained or obtainable by cleavage (for example by ADAM10 protease cleavage) at R101 of SEQ ID NO:1, or a fragment or variant thereof. For example, for human CD23 an exemplary soluble CD23 molecule comprises or corresponds to the sequence L102 to S321 of SEQ ID NO:1 (SEQ ID NO:5), or a fragment or variant thereof (or a corresponding or equivalent sequence in other forms of CD23, e.g. other species of CD23).

(SEQ ID NO: 5)
lksqdlels wnlnglqadl
ssfksqelne rneasdller lreevtklrm elqvssgfvc ntcpekwinf qrkcyyfgkg
tkqwvharya cddmegqlvs ihspeeqdfl tkhashtgsw iglrnldlkg efiwvdgshv
dysnwapgep tsrsqgedcv mmrgsgrwnd afcdrklgaw vcdrlatctp pasegsaesm
gpdsrpdpdg rlptpsaplh s

Constructs comprising the C-type lectin domain (CTLD) or C-type lectin head domain of CD23 are preferred, in particular constructs comprising at least 2 monomers comprising the C-type lectin domain (CTLD) or C-type lectin head domain of CD23.

The term head domain (or the C-type lectin head domain or C-type lectin domain or CTLD) of CD23 as referred to herein preferably refers to the sequence V159 to P290 of SEQ ID NO:1 (SEQ ID NO:6) or a fragment or variant thereof (or a corresponding or equivalent sequence in other forms of CD23, e.g. other species of CD23). A preferred CTLD refers to the sequence C160-C288 of SEQ ID NO:1 (SEQ ID NO:7) or a fragment or variant thereof (or a corresponding or equivalent sequence in other forms of CD23, e.g. other species of CD23). Another preferred CTLD refers to the sequence F170-L277 of SEQ ID NO:1 (SEQ ID NO:8) or a fragment or variant thereof (or a corresponding or equivalent sequence in other forms of CD23, e.g. other species of CD23).

(SEQ ID NO: 6)
vc ntcpekwinf qrkcyyfgkg
tkqwvharya cddmegqlvs ihspeeqdfl tkhashtgsw iglrnldlkg efiwvdgshv
dysnwapgep tsrsqgedcv mmrgsgrwnd afcdrklgaw vcdrlatctp
(SEQ ID NO: 7)
c ntcpekwinf qrkcyyfgkg
tkqwvharya cddmegqlvs ihspeeqdfl tkhashtgsw iglrnldlkg efiwvdgshv
dysnwapgep tsrsqgedcv mmrgsgrwnd afcdrklgaw vcdrlatc
(SEQ ID NO: 8)
f qrkcyyfgkg
tkqwvharya cddmegqlvs ihspeeqdfl tkhashtgsw iglrnldlkg efiwvdgshv
dysnwapgep tsrsqgedcv mmrgsgrwnd afcdrkl

Another exemplary soluble CD23 or molecule comprising the C-type lectin head domain of CD23 for use in the constructs of the present invention could comprise or correspond to the S155 fragment of CD23, which is obtained or obtainable by cleavage (for example by der p1 protease cleavage) at S155 of SEQ ID NO:1, or a fragment or variant thereof. For example, for human CD23 an exemplary such molecule comprises or corresponds to the sequence S156 to S321 of SEQ ID NO:1 (SEQ ID NO:9), or a fragment or variant thereof (or a corresponding or equivalent sequence in other forms of CD23, e.g. other species of CD23).

(SEQ ID NO: 9)
sgfvc ntcpekwinf qrkcyyfgkg
tkqwvharya cddmegqlvs ihspeeqdfl tkhashtgsw iglrnldlkg efiwvdgshv
dysnwapgep tsrsqgedcv mmrgsgrwnd afcdrklgaw vcdrlatctp pasegsaesm
gpdsrpdpdg rlptpsaplh s

Such constructs contain all or part of the head domain (or the C-type lectin head domain or C-type lectin domain or CTLD) of CD23 and preferably contain all of the head domain. Preferred constructs contain no or only a few additional residues of CD23, e.g. the stalk domain, e.g. up to 40, 35, 30, 25, 20, 15 or 10 additional residues of CD23, e.g. the stalk domain, e.g. up to 9, 8, 7, 6, 5, 4, 3, 2 or 1 additional residues of CD23, e.g. the stalk domain. Such additional residues would generally correspond to up to 40, 35, 30, 25, 20, 15, 10, etc., additional residues (consecutive residues) of CD23 located to the N-terminal side of (or before) S156, i.e. up to 10, 15, 20, 25, etc., of the residues immediately adjacent to S156 on the N-terminal side. It is preferred to avoid residues of the α-helical stalk as these may lead to self association and monomers are preferred. Thus, preferred constructs do not contain sufficient stalk residues to allow self association (e.g. dimerisation or trimerisation).

Preferred constructs containing some additional residues of the stalk domain have E133 of SEQ ID NO:1 (or a corresponding or equivalent sequence in other forms of CD23, e.g. other species of CD23) as the first CD23 residue used in such constructs (although other exemplary constructs may start at amino acids 134, 135, 136, 137, 138, 139, 140, 141, 142, 143, 144, 145, 146, 147, 148, 149, 150, 151, 152, 153, 154 or 155 of SEQ ID NO:1). In other words, in some embodiments, additional CD23 residues to the N-terminus of E133 (or before E133) of SEQ ID NO:1 (or a corresponding or equivalent sequence in other forms of CD23, e.g. other species of CD23), or before the other residues 134, 135, etc., are not included in the constructs. In other words E133 (or an amino acid after E133), or a corresponding residue, is the first CD23 residue used in such constructs.

Thus, for human CD23 another exemplary soluble CD23 or molecule comprising the C-type lectin head domain of CD23 and some of the stalk domain for use in the constructs of the present invention could comprise or correspond to the sequence E133 to A292 of SEQ ID NO:1 (SEQ ID NO:10), or a fragment or variant thereof (or a corresponding or equivalent sequence in other forms of CD23, e.g. other species of CD23).

(SEQ ID NO: 10)
easdller lreevtklrm elqvssgfvc ntcpekwinf qrkcyyfgkg
tkqwvharya cddmegqlvs ihspeeqdfl tkhashtgsw iglrnldlkg efiwvdgshv
dysnwapgep tsrsqgedcv mmrgsgrwnd afcdrklgaw vcdrlatctp pa

Thus, for human CD23 another exemplary soluble CD23 or molecule comprising the C-type lectin head domain of CD23 and some of the stalk domain for use in the constructs of the present invention comprises or corresponds to the sequence E133 to E298 of SEQ ID NO:1 (SEQ ID NO:11), or a fragment or variant thereof (or a corresponding or equivalent sequence in other forms of CD23, e.g. other species of CD23).

(SEQ ID NO: 11)
easdller lreevtklrm elqvssgfvc ntcpekwinf qrkcyyfgkg
tkqwvharya cddmegqlvs ihspeeqdfl tkhashtgsw iglrnldlkg efiwvdgshv
dysnwapgep tsrsqgedcv mmrgsgrwnd afcdrklgaw vcdrlatctp pasegsae

Thus, for human CD23 another exemplary soluble CD23 or molecule comprising the C-type lectin head domain of CD23 and some of the stalk domain for use in the constructs of the present invention comprises or corresponds to the sequence E133 to S321 of SEQ ID NO:1 (SEQ ID NO:12), or a fragment or variant thereof (or a corresponding or equivalent sequence in other forms of CD23, e.g. other species of CD23).

(SEQ ID NO: 12)
easdller lreevtklrm elqvssgfvc ntcpekwinf qrkcyyfgkg
tkqwvharya cddmegqlvs ihspeeqdfl tkhashtgsw iglrnldlkg efiwvdgshv
dysnwapgep tsrsqgedcv mmrgsgrwnd afcdrklgaw vcdrlatctp pasegsaesm
gpdsrpdpdg rlptpsaplh s

In other embodiments, additional CD23 residues to the N-terminus of S156 (or before S156) of SEQ ID NO:1 (or a corresponding or equivalent sequence in other forms of CD23, e.g. other species of CD23) are not included in the constructs. In other words S156 (or an amino acid after S156) or a corresponding residue is the first CD23 residue used in such constructs.

A preferred naturally occurring form of soluble CD23 or molecule comprising the C-type lectin head domain of CD23 for use in the invention comprises or corresponds to the derCD23 fragment which is obtained or obtainable by cleavage (for example by der p1 protease cleavage) at S155 and E298 of SEQ ID NO:1, or a fragment or variant thereof. For example, for human CD23 an exemplary such molecule comprises or corresponds to the sequence S156 to E298 of SEQ ID NO:1 (SEQ ID NO:13), or a fragment or variant thereof (or a corresponding or equivalent sequence in other forms of CD23, e.g. other species of CD23). The derCD23 fragment is monomeric in its native form and as stated elsewhere herein such monomeric fragments are preferred.

(SEQ ID NO: 13)
sgfvc ntcpekwinf qrkcyyfgkg
tkqwvharya cddmegqlvs ihspeeqdfl tkhashtgsw iglrnldlkg efiwvdgshv
dysnwapgep tsrsqgedcv mmrgsgrwnd afcdrkigaw vcdrlatctp pasegsae

Although such naturally occurring fragments of CD23 are convenient and in some embodiments preferred for use in the present invention, any appropriate fragments of soluble CD23 can be used in the constructs of the present invention.

For fragments (and variants thereof) as described herein, although preferred end (C-terminal) residues are given, such fragments (or variants) can end at any appropriate amino acid in the CD23 molecule, for example can end at any amino acid including, or after, L277, C288 or P290.

For fragments (and variants thereof) as described herein, although preferred start (N-terminal) residues are given, such fragments (or variants) can start at any appropriate amino acid in the CD23 molecule, for example in preferred embodiments can start at any amino acid including, or after, E133 or S156.

As described above, CD23 is the low affinity receptor for IgE and thus has the ability to bind to IgE, for example has the ability to bind or interact with the Cε2-4 part of the IgE Fc region, in particular the Cε3 part of the IgE Fc region. Any appropriate form of soluble CD23 or molecule comprising the CTLD of CD23 (or fragment or variant thereof) can be used in the constructs of the present invention providing that the ability to bind to IgE is retained or present. Thus, a preferred feature of soluble CD23 molecules or CTLDs of CD23 (or fragment or variant thereof) for use in the present invention is the presence of an IgE binding domain. IgE binding domains have been mapped in various CD23 molecules known in the art. For example, a preferred IgE binding domain is located between amino acids W184 to A279 in the human CD23a isoform as represented by SEQ ID NO:1. Thus, soluble CD23 molecules or CTLDs of CD23 or fragments or variants thereof comprising an IgE binding domain, for example comprising an IgE binding domain comprising or corresponding to the sequence located at amino acids W184 to A279 of SEQ ID NO:1 (SEQ ID NO:14), or IgE binding fragments or variants thereof (or a corresponding or equivalent sequence in other forms of CD23), are preferred.

Thus, soluble CD23 molecules or CTLDs of CD23 comprising these residues (wvharya cddmegqlvs ihspeeqdfl tkhashtgsw iglrnldlkg efiwvdgshv dysnwapgep tsrsqgedcv mmrgsgrwnd afcdrklga, SEQ ID No:14), or residues corresponding to these residues as found in other forms of CD23, e.g. in other (non-human) species of CD23, or fragments or mutated (or variant) versions thereof which retain the ability to bind IgE, are preferred.

Particularly key residues for IgE binding in the IgE binding domain of SEQ ID NO:1 have been identified as W184, R188, Y189, A190, L198, H202, I221, G222, R224, N225, L226, W234, V235, A271, C273, D274, K276 and A279 These residues are all located on a continuous surface on the lectin head which forms an IgE binding surface and thus preservation of enough of these residues such that the binding surface remains functional is likely to be important. Thus, any fragment, mutant or variant forms of soluble CD23 or CTLD of CD23 for use in the present invention preferably contain one or more, for example 2, 3, 4, 5, 6, 7, 8, 9, 10, 11, 12, 13, 14, 15, 16, 17, or more, and preferably all of these residues, or the equivalent residues in CD23 sequences other than SEQ ID NO:1, such that the soluble CD23 molecule can bind IgE.

Native monomeric CD23, for example the derCD23 fragment, can bind to IgE with an affinity of around 0.1-3 μM. Thus, it is preferred that any soluble CD23 molecule or molecule comprising the CTLD of CD23 (in particular monomers of such CD23 molecules) for use in the present invention can (e.g. individually) bind to IgE with a similar or improved affinity, for example an affinity of less than 20 μM, for example less than 15 μM, 10 μM, 5 μM, 4 μM, 3 μM, 2 μM or 1 μM. Forms of soluble CD23 which can bind to IgE with higher affinity are also contemplated, for example with affinities of less than 500, 400, 300, 200, 100, 50, 40, 30, 20, 10 or 1 nM. For example mutated or variant forms of soluble CD23 molecules as described herein can be selected to have such improved affinities for IgE.

Exemplary soluble CD23 molecules or molecules comprising the CTLD of CD23 (in particular monomers of such CD23 molecules), or fragments or variants thereof, for use in the present invention can bind to IgE with a high enough affinity/avidity to form a complex stable enough to prevent (or reduce or significantly reduce) binding of IgE to FcεRI, e.g under physiological conditions such as in serum. In embodiments where at least two monomers of soluble CD23 molecules or molecules comprising the CTLD of CD23 (or fragments or variants thereof) are present in the constructs, it is preferred that an improvement or increase in avidity of binding of the construct of the invention to IgE of at least (or up to) 1.5 fold, 2 fold, 5 fold, 10 fold, 50 fold, 100 fold, 200 fold, 300 fold, 400 fold, 500 fold, 750 fold, or 1000 fold, compared to that of a single monomer or the sum of the binding affinities of the individual monomers, is observed.

In some embodiments, it is preferred that the molecules (or monomers) of part a) of the construct, e.g. the CD23 based part of the construct, bind (e.g. individually) to IgE with a similar affinity to native monomeric CD23 (around native affinity), for example, with an affinity of between 0.1 μM and 20, 15, 10, 5, 4, 3, 2.5, 2, 1.5, 1 or 0.5 μM, e.g. 0.1-3 μM or 0.1-2 or 2.5 μM, or with an affinity between 0.5 μM and 20, 15, 10, 5, 4, 3, 2.5, 2, 1.5, or 1 μM, or with an affinity of between 1 μM and 20, 15, 10, 5, 4, 3, 2.5, or 2 μM, or with an affinity of between 2 μM and 20, 15, 10, 5, 4, or 3 μM, or with an affinity of between 3 or 4 μM and 20, 15, 10, or 5 or 4 μM. In other words molecules (or monomers) which bind IgE with μM affinities (low affinities) are sometimes preferred.

Any appropriate method of determining binding affinity (K_{D}) may be used. However, conveniently the K_{D} may be determined in a Surface Plasmon Resonance (SPR) assay (e.g. a BIAcore assay). Such assays can be designed in any appropriate way, for example an assay in which IgE-Fc is captured (or immobilised) to the chip (solid support), for example via an antibody to Fc (e.g. an anti-Fc Fab, e.g. an anti-IgE Fc Fab), and various concentrations (e.g. a dilution series, e.g. a doubling dilution series) of the relevant form of CD23 added to assess binding. Thus, the binding affinity (K_{D}) values as described above may be as determined in an SPR assay, for example as described above or elsewhere herein. A particularly preferred method is described in the Examples section herein.

In addition, soluble CD23 molecules or molecules comprising a CTLD of CD23 (or a fragment or variant thereof) for use in the invention should preferably have the ability not only to bind to IgE, for example bind in the Cε2-4 part, in particular the Cε3 part, of the IgE Fc region, or to recognise or interact with the CD23 binding site in the Cε2-4, in particular Cε3 part of the IgE Fc region, but also have the ability to inhibit, e.g. prevent or hinder or reduce, the binding of IgE to its high affinity receptor, FcεRI. Such inhibition can be by any mechanism, e.g. by steric hindrance. Preferably such inhibition involves the induction of an allosteric (conformational) change in IgE such that when it is bound to the soluble CD23 or molecule comprising a CTLD of CD23 (or fragments or variants thereof) in the constructs of the present invention it can no longer bind to high affinity receptor FcεRI (e.g. such binding is prevented or absent or undetectable or unmeasurable), or such binding is at least significantly or measurably reduced or inhibited, e.g. compared to when no construct is present. Thus, preferred soluble CD23 molecules or molecules comprising a CTLD of CD23 (or fragments or variants thereof) for use in the constructs of the present invention have the ability to bind to IgE (for example the Cε3 domain of the IgE Fc region) when it is in the closed conformation. Such binding can preferably then prevent or reduce the formation of the open conformation of IgE, or in other words can lock or maintain IgE in the closed conformation (Wurzburg et al., 2000, Immunity, 13(3):375-385). It is the open conformation of IgE (for example open conformation of the Cε3 domain of the IgE Fc region) which allows binding to the high affinity receptor FcεRI. Thus, the locking or maintaining of IgE into its closed conformation using the CD23 based parts of the constructs of the invention can prevent binding to FcεRI. Both steric hindrance and allosteric (or conformational) changes can be involved. Viewed alternatively the binding of IgE to FcεRI or FcεRII (CD23) can be regarded as mutually exclusive binding, i.e. a single molecule of IgE cannot bind to FcεRI and CD23 (FcεRII) at the same time.

Such allosteric changes, e.g. to inhibit IgE binding to FcεRI, can be induced by native CD23 molecules, including soluble CD23 molecules, and thus preferably this ability is retained or present in the soluble CD23 molecules or molecules comprising a CTLD of CD23 (or fragments or variants thereof) for use in the constructs of the present invention. It is also relevant to note that in its free-form, IgE maintains a closed conformation which is accessible to binding by CD23, including the CD23 based molecules present in the constructs of the invention. Thus, the soluble CD23 molecules or molecules comprising a CTLD of CD23 (or fragments or variants thereof) have the ability to bind to free (or free-form or circulating) IgE.

Such allosteric changes induced by CD23 and therefore by the constructs of the invention provide key differences over other anti-IgE therapeutics in the art. For example, many of these, such as Omalizumab and Ligelizumab, competitively block the binding of IgE to both CD23 and the high affinity receptor FcεRI by targeting the FcεRI binding site on IgE, whereas the constructs of the present invention target the CD23 binding site on IgE.

Thus, viewed alternatively, preferred soluble CD23 molecules or molecules comprising a CTLD of CD23 (or fragments or variants thereof) for use in the present invention have the ability to bind to (or interact with) the native CD23 binding site on IgE Fc. This binding site is described in the art (Borthakur et al., 2012, J. Biol. Chem. 287:31457-31461) and comprises residues from three discontinuous sequences (amino acids 405-407, 409-411 and 413 from the E-F helix, amino acids 377-380 from the C-D loop, and residue 436 from the C-terminal region, see Uniprot P01854). The ability of CD23 based molecules (or fragments or variants) to bind to this site could be tested by a person skilled in the art, for example by repeating the NMR-HSQC mapping study by Borthakur et al., 2012, supra., using 15N-labelled IgE-Cε3 domain and unlabelled CD23 or by using HDX (hydrogen-deuterium exchange) mass spectrometry.

Viewed alternatively, preferred soluble CD23 molecules or molecules comprising a CTLD of CD23 (or fragments or variants thereof) for use in the invention do not have the ability to bind IgE (or show insignificant or undetectable binding to IgE) when IgE is already bound to the high affinity receptor FcεRI. This is important not just from an efficacy standpoint, but also from a safety perspective, as, if the protein constructs of the invention retained the potential to bind to IgE when it was bound to its high affinity receptor FcεRI, then there might be the potential to cross-link IgE already bound to the high affinity receptor FcεRI on mast cells (or other cell types) and thereby cause a highly pro-inflammatory degranulation reaction. Thus, protein constructs of the invention (and CD23 based parts of the protein constructs of the invention) should preferably not be able to cross-link FcεRI when bound to IgE (for example should not be able to cross-link IgE, or bind to IgE, when it (IgE) is bound to FcεRI on cells). Indeed, this advantageous property is demonstrated by the constructs of the invention in the attached experimental Examples.

In some embodiments, the soluble CD23 molecules or molecules comprising a CTLD of CD23 (or fragments or variants thereof) for use in the constructs of the present invention also comprise or contain an integrin binding site and/or a CD21 binding site, for example one or more native integrin binding sites and/or CD21 binding sites. Put another way, the soluble CD23 molecules or molecules comprising a CTLD of CD23 (or fragments or variants thereof) for use in the constructs of the present invention can have the ability to bind to integrin and/or CD21 depending on the binding sites which are present. For human CD23, the essential amino acid residues for the integrin binding site are believed to be located at residues 172 to 174 of SEQ ID NO:1 and has the sequence RKC. However, equivalent or corresponding integrin binding sites in alternative forms of CD23, e.g. other species of CD23, will be readily identified or determined by a person skilled in the art. Similarly, the CD21 binding site in human CD23 is believed to be located at residues 294 to 298 or 293 to 298 of SEQ ID NO:1 and has the sequence EGSAE (SEQ ID NO:30) or SEGSAE (SEQ ID NO:24). However, equivalent or corresponding CD21 binding sites in alternative forms of CD23, e.g. other species of CD23, will be readily identified or determined by a person skilled in the art.

In other embodiments, the soluble CD23 molecules or molecules comprising a CTLD of CD23 (or fragments or variants thereof) for use in the constructs of the present invention will not comprise or contain an integrin binding site and/or a CD21 binding site, for example will not contain one or more of the native integrin binding sites and/or CD21 binding sites. Put another way, these soluble CD23 molecules or molecules comprising a CTLD of CD23 (or fragments or variants thereof) for use in the constructs of the present invention will not have the ability to bind (e.g. will show undetectable or insignificant binding) to integrin and/or CD21. Such forms of CD23 will be preferred in some circumstances, for example to prevent unwanted binding interactions. Thus, preferred part a) components of the constructs of the present invention do not comprise or contain a CD21 binding site. Other preferred part a) components of the constructs of the present invention do not comprise or contain an integrin binding site. Other preferred part a) components of the constructs of the present invention do not comprise or contain a CD21 binding site or an integrin binding site.

In some embodiments, the soluble CD23 molecules or molecules comprising a CTLD of CD23 (or fragments or variants thereof) for use in the constructs of the present invention also comprise or contain all or part of the C-terminal tail region of CD23. For human CD23, in addition to the CD21 binding site/binding region, the C-terminal tail region of CD23 is believed to comprise residues S299 to S321 of SEQ ID NO:1 and has the sequence as shown in SEQ ID NO:25. However, equivalent or corresponding C-terminal tail regions in alternative forms of CD23, e.g. other species of CD23, will be readily identified or determined by a person skilled in the art. Any number of amino acids of the C-terminal tail region may be included, for example at least 1, 2, 3, 4, 5, 10, 15 or 22 amino acids might be included, for example of SEQ ID NO:25. In other embodiments, the soluble CD23 molecules or molecules comprising a CTLD of CD23 (or fragments or variants thereof) for use in the constructs of the present invention will not contain any residues from the C-terminal tail region of CD23, e.g. will not contain any residues from or corresponding to SEQ ID NO:25. In other words the C-terminal tail region of CD23 is absent.

Soluble CD23 molecules or molecules comprising a CTLD of CD23 (or fragments or variants thereof) which do not comprise such sites can readily be developed or engineered by a person skilled in the art. For example, all or part of such sites can be removed by deletion of one or more residues making up the site or by mutation of one or more residues making up the site, such that the biological function (integrin binding or CD21 binding as appropriate) are disrupted, reduced or removed, preferably without affecting other functional properties of the starting or parent molecule, such as the various desired properties as described elsewhere herein. Thus, such molecules will be examples of variant or mutant CD23 molecules, or CD23 molecules which are substantially homologous to native soluble CD23 sequences.

In particular, for the CD21 site (or C-terminal tail region), a convenient way to produce a CD23 based molecule without a CD21 site (or C-terminal tail region) for use in the constructs of the present invention is to use a fragment of CD23 which is or corresponds to a CD23 molecule that has been truncated before the CD21 site (or C-terminal tail region). For example, for human CD23, the CD23 based molecules can be truncated at (and including), or before, S293 or A292 or P291 or P290 or C288. Truncation at (and including A292 or C288) is sometimes preferred. Thus, a preferred CD23 based molecule is or corresponds to S156 to A292 of SEQ ID NO:1 (SEQ ID NO:15). Another preferred CD23 based molecule is or corresponds to S156 to C288 of SEQ ID NO:1 (SEQ ID NO:31). Equally truncation within the CD21 binding site can be envisaged providing the ability to bind CD21 is removed. Thus, for human CD23, the CD23 based molecules can be truncated at (and including) E294, G295, S296 or A297. Truncations within the C-terminal tail region, e.g. between S299 and S321 for human CD23, or anywhere within the sequence corresponding to SEQ ID NO:25, are also contemplated.

As in other embodiments of the invention corresponding or equivalent sequences in other forms of CD23, e.g. other species of CD23, can equally be used. For example, a canine sequence which corresponds to S156 to C288 of SEQ ID NO:1 is outlined below (SEQ ID NO:32). Thus, preferred constructs of the invention can comprise this canine sequence, or a fragment or variant thereof as described elsewhere herein, including for example an amino acid sequence with a sequence identity of at least 70%, etc., thereto as described elsewhere herein.

(SEQ ID NO: 15)
sgfvc ntcpekwinf qrkcyyfgkg
tkqwvharya cddmegqlvs ihspeeqdfl tkhashtgsw iglrnldlkg efiwvdgshv
dysnwapgep tsrsqgedcv mmrgsgrwnd afcdrklgaw vcdrlatctp pa
(SEQ ID NO: 31)
sgfvc ntcpekwinf qrkcyyfgkg
tkqwvharya cddmegqlvs ihspeeqdfl tkhashtgsw iglrnldlkg efiwvdgshv
dysnwapgep tsrsqgedcv mmrgsgrwnd afcdrklgaw vcdrlatc
(SEQ ID NO: 32)
NGSECNTCPEKWLNFQRKCYYFGEEPKKWIQARFACSKLQGRLASIHSQEEQDFLA
RYANKKGTWIGLRDLDREGEFIWMDENPLNYSNWRPGEPNNGGQGEDCVMMQGS
GQWNDAFCGSSLDGWVCDRLATC

The removal of such sites is optional and such sites may not even be present in all forms of CD23 that are contemplated for use in the present invention. For example murine CD23 does not have the ability to bind to CD21 and does not contain the residues required for binding to CD21 (does not contain a CD21 binding site).

As described in more detail elsewhere herein, CD23 binding to IgE is calcium dependent/calcium sensitive (Yuan et al., 2013, J. Biol. Chem. 288(30): 21667-21677). Thus, binding (e.g. good or stable binding) of CD23 to IgE takes place in conditions of high calcium, e.g. high physiological calcium, for example at levels of calcium found extracellularly or interstitially, e.g. within tissues or in serum or blood. Such levels of calcium (or calcium ion concentration) are generally around 2 mM, e.g. 1.0 to 2.5 mM or 1.0 to 2.0 mM. In contrast, binding of CD23 to IgE is highly reduced or not present or absent (e.g. essentially absent) in conditions of low calcium, e.g. low physiological calcium, for example at levels found in acidic compartments of the body such as intracellular acidic compartments such as endosomes. Such low levels of calcium (calcium ion concentrations) are generally between 30 to 1000 times lower than the high calcium levels (calcium ion concentrations), for example around 3-30 μM, although levels as low as 100 or 500 nm have been reported. Thus, when CD23 that is bound to IgE (e.g. complexes of CD23-IgE) transitions from a high calcium to a low calcium environment, for example when taken up into endosomes from the serum or tissues, the IgE is released, e.g. rapidly released, from CD23.

This calcium dependence of CD23 binding to IgE is a highly important and advantageous feature behind the present invention. Thus, any soluble CD23 molecule or molecules comprising a CTLD of CD23 (or fragments or variants thereof) used in the constructs of the present invention should preferably retain or have the ability to bind calcium and also retain or have the ability to bind, e.g. stably bind, IgE under conditions of high calcium or high physiological calcium, for example serum calcium levels as described above, and show lower, e.g. significantly or measurably lower, or no binding, or no significant binding, of IgE under conditions of low calcium or low physiological calcium, for example endosomal calcium levels as described above. References herein to the presence of calcium or calcium levels, e.g. high calcium levels or low calcium levels, etc., also include reference to the presence of calcium ions or calcium ion concentrations.

Thus, in some protein constructs of the invention said binding of part a) of the construct to IgE is reduced at endosomal calcium levels compared to serum calcium levels.

In human CD23, the residues believed to be involved in calcium dependent binding to IgE are Thr251, Ser252, Glu249, Asp270, Asn269 in loop 4 and Asn225 and Asp258 in loop 1. Thus, in any variant CD23 based molecules used in the constructs of the present invention, it is preferred that one or more of these residues, preferably all of these residues are retained or present.

In other variant CD23 based molecules for use in the constructs of the present invention it may be possible to increase the binding affinity of CD23 for IgE by altering or mutating the calcium binding site and increasing the binding affinity for IgE. However, as low or moderate affinity interactions (individual affinity interactions) between IgE and CD23 are preferred in some embodiments, equally it is preferred that variant or mutated CD23 molecules do not show increased calcium binding which, for example, results in calcium induced increased affinity for IgE. Thus, in general, in some embodiments, mutants or variant CD23 based molecules with increased affinity to IgE (e.g. high affinity mutants or variants, e.g. compared with a native or wild-type or starting molecule) or increased calcium binding (e.g. compared with a native or wild-type or starting molecule) are not used in the constructs of the invention. For example, preferred CD23 based molecules for use in the invention do not contain or comprise a D to E mutation at residue 258 of human CD23 (or corresponding residues in other forms of CD23, e.g. in other species of CD23), which is thought to increase calcium binding and hence increase affinity for IgE). Preferred CD23 based molecules for use in the invention thus show similar calcium binding (e.g. not significantly different levels of calcium binding) as native or wild-type CD23 molecules. Other preferred CD23 based molecules for use in the invention thus show similar IgE binding (e.g. not significantly different levels of IgE binding) as native or wild-type CD23 molecules, e.g. with affinity levels as described elsewhere herein.

Other preferred features of the construct could be readily conceived by a person skilled in the art and might for example include the removal (or non-inclusion in the constructs) of glycosylation sites, or other sites subject to post-translational modification, for example to improve production in non-mammalian hosts, and also the removal (or non-inclusion in the constructs) of protease cleavage sites, for example to avoid unwanted cleavage or processing (e.g. proteolytic cleavage or processing) of the construct when producing or administering the construct. Such sites can be readily identified and removed by a person skilled in the art using standard techniques.

Preferred protein constructs of the present invention comprise or contain one or more, two or more, three of more, or all of the following features as described in more detail elsewhere herein:
i) No CD21 binding site;
ii) No integrin binding site;
iii) No glycosylation sites;
iv) No protease cleavage sites.

These features can be present in the constructs in addition to the preferred functional features as described elsewhere herein such as calcium sensitive binding of part a) of the constructs to IgE in the serum versus endosomes, together with FcRn binding as mediated by part b) of the constructs.

It can be seen from the above that fragments (functional fragments) or variants (functional variants) of CD23 molecules are also appropriate soluble CD23 molecules or molecules comprising a CTLD of CD23, for use in the present invention.

Appropriate fragments or variants (appropriate soluble CD23 molecules or molecules comprising a CTLD of CD23), for use in the present invention can be of any length provided one or more of the appropriate described functional features, e.g. IgE binding, etc., as described elsewhere herein are retained. Fragments are generally shorter in length than the original or parent sequence. Exemplary lengths/fragment lengths might be at least 50, 60, 70, 80, 100, 125, 140,150, 175, 180, 185, 190, 195, 200, 205, 210, 215, 220, 225 or 250 amino acids in length. Alternatively viewed, exemplary lengths/fragment lengths might be up to 60, 70, 80, 100, 125, 130, 135, 140, 145, 150, 155, 160, 165, 170,175, 200, 225, 250, 275, 300 or 350 amino acids in length. Thus, exemplary lengths/fragment lengths might be 50, 60, 70, 80, 90, 100, 110, 120, 125, or 130 amino acids to 140, 145, 150, 155, 160, 165, 170, 175, 180, 185, 190, 195, 200, 225, 250, 275 or 300 or 350 amino acids long. As will be clear from other disclosures herein, full length native or wild-type CD23 molecules (e.g. of SEQ ID NO:1), or equivalents in other species, are not used in the constructs of the invention, for example extracellular regions of CD23 are generally preferred for use in the constructs of the present invention. In particular, the cytoplasmic region and transmembrane region of CD23 are not desirable for inclusion. A full length stalk region is also not desirable. However, in some embodiments the use of CD23 sequences (e.g. fragments of CD23) corresponding to sequences as found in native or wild-type CD23 is preferred.

Appropriate variants (functional variants) of soluble CD23 molecules or molecules comprising a CTLD of CD23, for use in the present invention can conveniently be defined by sequence homology and CD23 sequences that are substantially homologous to the various sequences of CD23 molecules as defined herein can readily be used in the invention providing that the appropriate functional characteristics of the original (or parent) CD23 molecule are retained.

Appropriate variants (or mutated sequences or substantially homologous sequences) might comprise or consist of an amino acid sequence with a sequence identity of at least 70%, 75% or 80% to the above-mentioned CD23 sequences, such as at least 80%, 81%, 82%, 83%, 84%, 85%, 86%, 87%, 88%, 89%, 90%, 91%, 92%, 93%, 94%, 95%, 96%, 97%, 98%, or 99% identity. These variant sequences should retain or have the appropriate functional properties of CD23 molecules as defined elsewhere herein. Functional truncations or fragments of these sequences (or these homologous sequences) could also be used providing the appropriate functional properties are retained. Other preferred examples of mutated or variant soluble CD23 molecules or molecules comprising a CTLD of CD23, are sequences containing up to 20, e.g. up to 18, 15, 12, 10, 9, 8, 7, 6, 5, 4, 3, 2, or 1 altered amino acids in the above CD23 sequences.

% identity may be assessed by any convenient method. However, for determining the degree of homology between sequences, computer programs that make multiple alignments of sequences are useful, for instance Clustal W (Thompson et al., Nucleic Acids Res., 22:4673-4680, 1994). Other methods to calculate the percentage identity between two amino acid sequences are generally art recognized and include, for example, those described by Carillo and Lipton (SIAM J. Applied Math., 48:1073, 1988).

Generally, computer programs will be employed for such calculations. Programs that compare and align pairs of sequences, like ALIGN (Myers and Miller, CABIOS, 4:11-17, 1988), FASTA (Pearson, Methods in Enzymology, 183:63-98, 1990) and gapped BLAST (Altschul et al., Nucleic Acids Res., 25:3389-3402, 1997), or BLASTP (Devereux et al., Nucleic Acids Res., 12:387, 1984) are also useful for this purpose.

By way of providing a reference point, sequences according to the present invention having at least 70%, etc., identity may be determined using the ALIGN program with default parameters (for instance available on Internet at the GENESTREAM network server, IGH, Montpellier, France).

In all aspects of the invention as described herein, reference to a soluble CD23 molecule or a molecule comprising a CTLD of CD23, can equally refer to a fragment or variant of soluble CD23 or a fragment or variant of molecules comprising a CTLD of CD23 (as appropriate), as described herein.

The soluble CD23 component (or fragment or variant thereof) or molecules comprising a CTLD of CD23 (or fragments or variants thereof), of the protein construct of the present invention provides the ability of the construct to bind to IgE. One molecule of CD23 (or fragment or variant thereof) can confer this ability, for example if the binding affinity for IgE is sufficient. In this regard, it is known in the art that monomeric CD23 can bind to IgE with affinity (K_{d}) in the region of 0.1-3 μM. Thus, single (or monomeric) CD23 molecules that have the ability to bind IgE with a K_{d} of about or less than 20, 15, 10, 5, 4, 3, 2, or 1 μM, or 500, 400, 300, 200, 100, 50, 40, 30, 20, 10 or 1 nM can be used, for example as described elsewhere herein. In embodiments of the invention where a fragment or variant of CD23 (or CTLD of CD23) is used, for example a sequence with a certain sequence identity to CD23 (or CTLD), then native soluble CD23 molecules might be modified such that they have an improved binding affinity but retain endosomal sensitivity (e.g. to calcium). Equally however, in some embodiments, native (or near native, or low) binding affinities are preferred and exemplary such affinities are described elsewhere herein.

Preferably however, two or more, for example two, molecules of soluble CD23 or molecules comprising a CTLD of CD23 (or fragments or variants thereof), are provided in the constructs of the invention. More preferably, two or more, for example two, monomers, or more than two monomers (e.g. 4, 6, 8 or 10 monomers) are provided. Thus, preferred protein constructs have two (or at least 2) binding sites for IgE, preferably provided by two (or at least two) monomers.

Where two or more molecules (or monomers) of CD23 are present then the protein constructs are generally and preferably designed such that the individual CD23 based molecules or monomers are separated such that each molecule or monomer can bind to IgE, thereby allowing an overall increase in binding affinity for IgE by virtue of an avidity effect (for example where two molecules or monomers of CD23 bind to one molecule of IgE, for example by way of one CD23 molecule or monomer binding to one chain of an IgE Fc and the other CD23 molecule or monomer binding to the other chain of the same IgE Fc, or, put another way, where two (or both) CD23 molecules (or monomers), or CD23 heads, can engage both CD23 binding domains from each chain of a single IgE Fc), e.g. by co-operative binding resulting in improved binding avidity. This type of interaction between the constructs of the invention and IgE molecules, is also referred to herein as cis-binding. Alternatively, such constructs of the invention where two or more molecules (or monomers) of CD23 are present can allow more than one IgE molecule to be bound to the construct of the invention, for example by a construct of the invention binding to two individual IgE molecules. This type of interaction between the constructs of the invention and IgE molecules is also referred to herein as trans-binding, which can in turn permit the formation of higher order structures or complexes such as higher order oligomers. Such interactions where higher order structures are formed can also result in improved binding avidity. Preferred interactions have both (in embodiments where two CD23 molecules are present) or all the CD23 molecules in the construct bound to IgE.

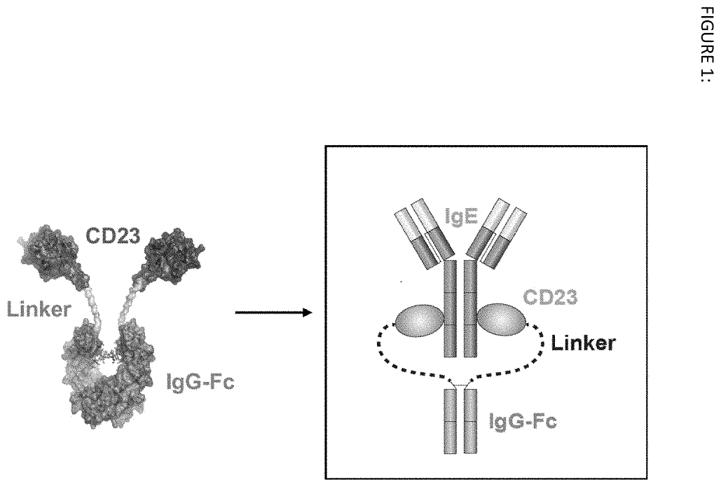

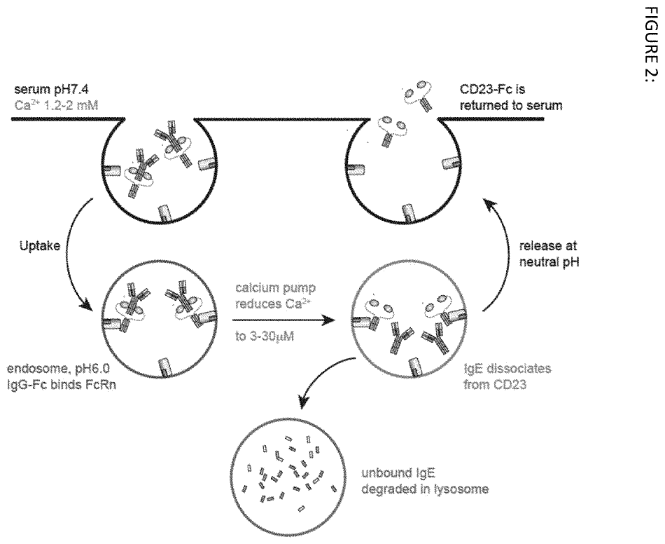

Alternative, or additional modes of co-operative binding may comprise >1 CD23 monomer binding to IgE Fc (e.g. multiple IgE Fc/multiple IgE molecules) such that all the CD23 binding sites on IgE (or the multiple IgEs) are occupied as a higher order form (higher order oligomer) resulting in a high avidity interaction with an apparent affinity (functional affinity, relative affinity or overall affinity) of binding to IgE that is greater, preferably significantly greater, than a monomer alone or the sum of binding affinities of the individual monomers in the structure. For example, it is known that two molecules of CD23 (for example two molecules of derCD23 or other CTLD of CD23) can bind to a single molecule of IgE or the same IgE Fc (see also the FIG. 1 schematic, which illustrates such cis-interactions) and thus, preferred constructs of the invention can replicate this.

Preferably where two or more molecules (preferably monomers) of CD23 are present, this allows for at least one molecule of CD23 to bind to one of the chains making up the IgE Fc region and at least one other molecule of CD23 in the construct to bind to the other chain making up the same IgE Fc region (e.g. via a cis-interaction), thereby allowing an avidity effect to improve binding affinity. Preferred constructs of the invention are bivalent in that they contain two molecules (preferably monomers) of CD23 and hence two IgE binding sites. Simple trans-binding interactions as described above (e.g. one construct of the invention binding to two individual IgE molecules) can also occur. Higher order structures or oligomers are however also contemplated and can also be formed from constructs containing two molecules of CD23, for example by way of co-operative binding of CD23 monomers to IgE Fc such that all CD23 binding sites on IgE are occupied. Such structures can for example form a ring-like or closed structure where there are no free CD23 binding sites in the IgE molecules present in the structure as they are all bound to CD23. The formation of such structures would also result in a high avidity interaction with a functional affinity of binding to IgE that is greater, preferably significantly greater, than a monomer alone or the sum of binding affinities of individual monomers in the structure. For example, two or three protein constructs of the invention, each with 2 monomers (or molecules) of CD23, can interact (link) by binding to IgE-Fc regions and form a ring-like structure with two or three molecules of IgE, respectively. Larger ring-like structures may also be formed in which generally an equal number of molecules of the construct of the invention and IgE will be present.

Such structures can for example form when the constructs of the invention have two CD23 molecules (monomers) and all the CD23 binding sites on IgE are occupied.

Where two or more molecules (preferably monomers) of CD23 are used, then preferably they are the same or identical molecules or monomers (for example can be referred to as a “pair” or multiple “pairs”, e.g. multiple identical “pairs”, of molecules or monomers).

Thus, in preferred constructs of the invention, the CD23 molecules, preferably the CD23 monomers, are spatially separated such that they can each bind one chain of the same (a single) IgE Fc dimer, i.e. one of the molecules (preferably monomer) binds to one chain of an IgE Fc dimer and the other molecule (preferably monomer) binds to the other chain of the same IgE Fc dimer. Put another way 1:1 whole molecule stoichiometry is observed. Thus, preferably the overall binding affinity (avidity) of the constructs of the invention for IgE where two molecules (preferably monomers) of CD23 are used, is increased (or improved), preferably significantly increased (or improved), than the binding affinity observed when the same single molecule (or monomer) is used. More preferably, the overall binding affinity (avidity) of the constructs of the invention for IgE where two molecules (preferably monomers) of CD23 are used, is increased (or improved), preferably significantly increased (or improved), than the sum of the binding affinities observed when the same single molecule (or monomer) is used. Similar increases in overall binding affinity also apply to constructs where more than two molecules (preferably monomers) of CD23 are used, e.g. when higher order structures or oligomers are formed as described elsewhere herein.

Preferred constructs of the invention use monomeric forms of CD23. In other words, the constructs of the invention preferably do not comprise dimers or trimers or other oligomers (e.g. homodimers, homotrimers, or other homooligomers or homomultimers) of CD23. The terms dimer, trimer, oligomer, etc., as used herein, refer to molecules which are physically associated or self-associated. Thus, in preferred constructs of the invention using monomers of CD23, the individual CD23 molecules in the construct are not directly physically associated with each other or are not directly physically interacting with each other or are not self-associated, for example in a dimer or trimer, and are present as separate entities which are each free to bind to IgE, in particular to bind to a single IgE molecule such that at least one monomer of CD23 in the construct binds to each of the two chains of the IgE Fc (e.g. by cis-interactions), or to bind to multiple IgE molecules (e.g. linking two free (or soluble) IgE molecules or forming other higher order constructs, e.g. by trans-interactions) such that at least one monomer of CD23 in the construct binds to a chain of IgE Fc and at least one other monomer of CD23 in the construct binds to a chain of a different IgE Fc, as disclosed elsewhere herein.

Preferred constructs of the invention can thus be regarded as biparatopic in that two epitopes on a single IgE molecule (generally two identical epitopes, one on each chain of a single IgE Fc), can be bound by a single construct of the invention when it has two or more monomers (or molecules) of CD23. Other preferred constructs may allow for bivalent binding, for example to two individual IgE molecules, to permit the formation of higher order structures or complexes such as higher order oligomers. Mixtures of these forms, and indeed any of the other forms described herein, may also be formed.

Thus, with the preferred protein constructs of the invention, each individual CD23 monomer of a pair of CD23 monomers present in the construct can simultaneously interact with the same target molecule of IgE. Whilst each single binding interaction between CD23 and IgE may be readily broken (depending on the affinity of interaction, for example when lower affinity (e.g. native) interactions are involved), when both members of the pair are interacting with the IgE antigen at the same time, the overall effect is synergistic, strong binding of the pair of CD23 monomers to IgE, in particular under physiological conditions, e.g. physiological pH, or for example in serum. In addition, when a single binding interaction is broken, the existence of the other interaction means that the IgE target molecule does not diffuse away thereby meaning that the broken binding interaction is likely to be reinstated (e.g. due to avidity). Similar interactions are also envisaged with higher order structures as described elsewhere herein.

Thus, in embodiments of the present invention where the individual CD23 monomers (or molecules) bind to target antigen (IgE) with low or moderate affinity, although the individual interactions of the CD23 monomers with target antigen (IgE) are low affinity or weak, the fact that there is a pair of CD23 monomers each member of which is interacting with the target antigen (IgE) with a low affinity or weak interaction (i.e. there are multiple individual weak interactions in a single construct), means that the overall interaction with a single target IgE molecule has the important advantageous feature of being high avidity, i.e. high overall affinity through avidity, or is of high potency in terms of the ability to inhibit the natural function of the target antigen, e.g. its ability to bind ligand, e.g. the ability of IgE to bind to its high affinity receptor, FcεRI, i.e. can inhibit target-ligand interactions with high potency. Similar (high overall affinity through avidity) interactions are also envisaged with higher order structures as described elsewhere herein.

Thus, in the preferred protein constructs of the present invention (and particularly in embodiments where the individual CD23 monomers bind to target IgE with low or moderate affinity, e.g. around native affinity of monomer binding to IgE as described elsewhere herein), an overall increase or improvement (or preferably a synergistic increase or improvement) in binding affinity (avidity) for IgE is observed when both members of the pair of CD23 monomers bind to the same target IgE molecule, as opposed to a single member of the pair (a single monomeric CD23) being bound. Such an overall increase includes any measurable increase, preferably a significant increase, more preferably a statistically significant increase (e.g. with a probability value of <0.05). For example, the overall binding affinity for IgE may be increased (or improved) by greater than one fold, e.g. at least 1.5 fold, 2 fold, 5 fold, 10 fold, 100 fold, 200 fold, 300 fold, 400 fold, 500 fold, 750 fold, or 1000 fold, e.g. by at least (or up to) 5 fold, 10 fold, 20 fold, 30 fold, 40 fold, 50 fold, 60 fold, 70 fold, 80 fold, 90 fold, 100 fold, 200 fold, 300 fold, 400 fold, 500 fold, 750 fold, or 1000 fold, when two CD23 monomers of a pair of CD23 monomers in a single construct are bound to the same target IgE molecule compared to when a single monomer of the pair is bound (or compared to the sum of binding affinities of the individual monomers). These increases or improvements should be observed at physiological pH (e.g. at or around pH 7.4) and/or at physiological calcium levels, as described elsewhere herein. This overall increase (or improvement) in binding affinity can readily be tested using constructs in which two monomers of the CD23 monomer pair are present versus constructs where only a single monomer (i.e. one member of the pair) is present and measuring and comparing the binding affinity for target antigen. By synergistic increase or improvement it is meant that the overall (combined) binding affinity for target antigen (IgE) when both members of a pair of CD23 monomers bind to the same target antigen simultaneously is greater than the sum of the individual binding affinities of each CD23 monomer of the pair to target antigen.

Viewed another way, by a synergistic increase or improvement it is meant that the overall binding affinity for target antigen (IgE) is increased (or improved) by greater than 1 fold, e.g. at least 1.5 fold, or 2 fold (e.g. with values as described above) when both members of a pair of CD23 monomers are bound to the same target antigen (IgE) compared to when a single CD23 monomer of the pair is bound. Similar increases and improvements in overall binding affinity (avidity) are also envisaged with higher order structures as described elsewhere herein, for example higher order structures which contain at least two molecules of the construct of the invention and at least two molecules of IgE (typically the same (or equal) number of both) and wherein structures, e.g. ring-like structures, are formed in which all the CD23 binding sites on IgE are occupied.

In other preferred protein constructs of the invention, more than one pair of CD23 molecules, preferably monomers, can be used. These multiple pairs may be the same as the first pair, or may be different pairs. Thus, protein constructs of the invention, can for example have or comprise four, six, eight or ten individual CD23 molecules, preferably monomers. These multiple pairs would be arranged appropriately such that for example each pair could bind a separate IgE molecule (e.g. a single IgE molecule, e.g. by cis-binding), or could form higher order structures or oligomers as described elsewhere herein. For example, in embodiments where four (e.g. 2 pairs) of individual CD23 molecules, preferably monomers, are used, two (e.g. 1 pair) individual CD23 molecules, preferably monomers, can be present at one end (e.g. the N-termini) of the construct, and another two (e.g. the second pair) individual CD23 molecules, preferably monomers, can be present at the other end (e.g. the C-termini) of the construct. Alternatively, in other embodiments where four (e.g. 2 pairs) of individual CD23 molecules, preferably monomers, are used, both pairs of individual CD23 molecules, preferably monomers, can be present at the same end (e.g. the N-terminus or C-terminus) of the construct, for example in a spatial configuration (for example where both members of a pair are linked together on the same polypeptide chain, or where both members of a pair are on different polypeptide chains), which allows each pair to interact with a single molecule of IgE. Such constructs can also form higher order structures or oligomers, e.g. by cooperative binding as described elsewhere herein.

In preferred embodiments of the invention, at least one of the individual molecules of sCD23 or sCD23 fragments or variants, or molecules comprising a CTLD of CD23 (or fragments or variants thereof), is engineered or selected so that the binding of the sCD23 or sCD23 fragment or variant, or molecules comprising a CTLD of CD23 (or fragments or variants thereof), to target antigen (IgE) is sensitive to endosomal conditions (conditions found within cellular endosomes). By “sensitive to endosomal conditions”, it is meant that the binding of the molecules of CD23 or CD23 fragments or variants (or pairs or multiple molecules thereof where two or more molecules, preferably monomers, are present in the constructs) to target antigen (IgE) can be disrupted or at least weakened or reduced under conditions found in the cellular endosome. The below discussion focuses on calcium sensitivity, i.e. molecules which bind to IgE under conditions of high calcium or serum calcium or physiological calcium as described elsewhere herein and show reduced binding to IgE under endosomal calcium conditions or low calcium as described elsewhere herein. However, sensitivity to other endosomal conditions could be used, e.g. pH sensitivity, and appropriate molecules for part a) of the constructs could be those which bind to IgE under conditions of physiological pH (e.g. pH 7.4) as described elsewhere herein and show reduced binding to IgE under endosomal pH conditions as described elsewhere herein (e.g. at pH 6.0 or 6.5).

In particular, the interaction between individual molecules of CD23 or CD23 fragments or variants (or pairs or multiple molecules thereof where two or more molecules, preferably monomers, are present in the constructs) and IgE can be sensitive to changes in calcium levels, for example the interaction is strong or stable in serum where the calcium level is ˜2 mM but is less stable or weakened or disrupted or reduced (for example is measurably reduced or significantly reduced, e.g. with probability value of <0.05) when calcium ion (Ca ²⁺) concentration falls to levels substantially less than 2 mM, e.g. to levels typically found in a mammalian endosome, for example between 3 and 30 μM or between 30 and 300 μM calcium. Put another way, the interaction between individual molecules of sCD23 or sCD23 fragments or variants (or pairs or multiple molecules thereof where two or more molecules, preferably monomers, are present in the constructs) and IgE is strong or stable at circulatory (serum) calcium concentrations or tissue/interstitial calcium concentrations in general, but is less stable or weakened or disrupted or reduced (for example is measurably reduced or significantly reduced, e.g. with probability value of <0.05) at low endosomal calcium concentrations, or under endosomal conditions in general.

This feature can advantageously allow recycling of the protein construct of the invention through the endosome. In such embodiments, the loaded protein construct, i.e. the protein construct of the invention when individual molecules of CD23 or CD23 fragments or variants (or pairs or multiple molecules thereof where two or more molecules, preferably monomers, are present in the constructs) are bound to IgE target antigen, enters or is internalised into the endosomal pathway, after which the IgE bound to the individual molecules of CD23 or CD23 fragments or variants (or pairs or multiple molecules thereof where two or more molecules, preferably monomers, are present in the constructs) is released or disassociates from the protein construct under endosomal conditions and the unloaded or empty protein construct is recycled back into the circulation to capture more target antigen (IgE) and thereby greatly enhance the in vivo half-life of the protein construct.

Thus, in these embodiments, the interaction between an individual molecule of CD23 or CD23 fragments or variants (or pairs or multiple molecules thereof where two or more molecules, preferably monomers, are present in the constructs) and its target antigen (IgE) has to be weakened sufficiently upon entry of the protein construct of the invention to the endosomes or endosomal pathway such that at least some of the target antigen (IgE) can be released or dissociates. The individual molecules of CD23 or CD23 fragments or variants (or pairs or multiple molecules thereof where two or more molecules, preferably monomers, are present in the constructs) for use in such protein constructs can be selected accordingly, for example by assaying for the binding to IgE at serum pH, e.g. at or around pH 7.4 and at normal calcium levels found in serum (e.g. at or around 2 mM or 1 mM as described elsewhere herein) or ideological calcium ion concentrations found in serum, and comparing it to the binding of individual molecules of CD23 or CD23 fragments or variants (or pairs or multiple molecules thereof where two or more molecules, preferably monomers, are present in the constructs) to IgE at endosomal pHs and/or calcium concentrations such as those described elsewhere herein, and identifying individual molecules of CD23 or CD23 fragments or variants (or pairs or multiple molecules thereof where two or more molecules, preferably monomers, are present in the constructs) for which the binding to target antigen (IgE) at the higher calcium concentration (or serum pH) is measurably higher (preferably significantly higher, e.g. with a probability value of <0.05) than the binding at the endosomal calcium concentration (or endosomal pH level). However, the use of individual molecules of CD23 or CD23 fragments or variants (or pairs or multiple molecules thereof where two or more molecules, preferably monomers, are present in the constructs) with low or moderate affinity for target antigen (IgE) at physiologically normal serum calcium concentrations are preferred in such embodiments as the individual interactions with target antigens (IgE) are weaker and therefore more readily disrupted or weakened under endosomal conditions, e.g. lower calcium levels (Ca²⁺ ions).

Thus, for this embodiment it is important that the individual molecules of CD23 or CD23 fragments or variants (or pairs or multiple molecules thereof where two or more molecules, preferably monomers, are present in the constructs) can bind to target antigen (IgE) with high avidity (overall affinity, relative affinity, functional affinity) at physiological serum or interstitial tissue calcium concentrations and to release or dissociate from the target antigen (IgE) at calcium concentrations typically found in endosomes, e.g. a mammalian endosome.

Thus, the binding interaction between individual molecules of CD23 or CD23 fragments or variants (or pairs or multiple molecules thereof where two or more molecules, preferably monomers, are present in the constructs) and IgE must be sufficiently stable at serum calcium concentration as discussed above, but the binding must be significantly or sufficiently weakened at endosomal calcium concentration, as discussed above, to allow the release of bound IgE or a proportion (preferably a measurable or significant proportion) of bound IgE.

Appropriate individual molecules of CD23 or CD23 fragments or variants (or pairs or multiple molecules thereof where two or more molecules, preferably monomers, are present in the constructs) will be those for which the binding affinity to IgE is significantly reduced (e.g. with a probability value of <0.05), for example at a calcium concentration of at or around 300 μM, or at a calcium concentration of less than 300 μM and greater than 100 μM, or at a calcium concentration of less than 100 μM and greater than 10 μM, or at a calcium concentration of less than 10 μM and greater than 0.1 μM. Preferably a complete loss of binding (or almost no capability of binding or no significant binding) is observed when endosomal calcium concentrations are used (e.g. 3 to 30 μM). However, more important is that the reduction in binding to IgE at the lower calcium concentration is sufficient to allow at least some and preferably a significant proportion of the target antigen (IgE) to dissociate, preferably rapidly dissociate, from the individual molecules of CD23 or CD23 fragments or variants (or pairs or multiple molecules thereof where two or more molecules, preferably monomers, are present in the constructs).

By way of example, appropriate calcium sensitive individual molecules of CD23 or CD23 fragments or variants (or pairs or multiple molecules thereof where two or more molecules, preferably monomers, are present in the constructs) might be those for which the binding affinity of the individual molecules of CD23 or CD23 fragments or variants (or pairs or multiple molecules thereof where two or more molecules, preferably monomers, are present in the constructs) to target antigen (IgE) at the lower calcium is reduced by at least (or up to) 5 fold, 10 fold, 20 fold, 30 fold, 40 fold, 50 fold, 75 fold, 100 fold, 200 fold, 300 fold, 400 fold, 500 fold, or 1000 fold, or more, compared to the binding affinity to target antigen (IgE) at normal physiological calcium levels as described elsewhere herein (e.g. at or around 2 mM calcium). Ideally the Kd of the individual molecules of CD23 or CD23 fragments or variants (or pairs or multiple molecules thereof where two or more molecules, preferably monomers, are present in the constructs) for target antigen (IgE) at the lower calcium would be in the high μM or mM range, for example 10 to 500 μM, or 500 to 1000 μM, or 1 to 100 mM.

In preferred embodiments the calcium sensitivity is reversible, i.e. the binding affinity is recovered once the calcium is increased back to the physiological conditions (e.g. around 2 mM calcium).

Thus, in some protein constructs of the invention said binding of part a) of the construct to IgE is reduced at endosomal pH levels (e.g. at pH 6.0 or 6.5, or other endosomal pH levels as described elsewhere herein) or endosomal calcium levels compared to serum pH levels (e.g. pH 7.4) or serum calcium levels. In some protein constructs of the invention said binding of part a) of the construct to IgE is reduced at pH 6.0 or 6.5 compared to pH 7.4.

The CD23 molecules for use in the constructs of the present invention can be obtained from or be derived from or can correspond to CD23 from any source or species, or can be a fragment or variant thereof. Preferred sources are mammalian, and any appropriate mammalian source may be used, for example humans or any livestock, domestic or laboratory animal. Specific examples include mice, rats, pigs, cats, dogs, sheep, rabbits, horses, cows and non-human primates (e.g. cynomolgus monkey). Thus, the CD23 molecules (e.g. the CTLDs) for use in the constructs of the present invention are or correspond to mammalian CD23 molecules such as those outlined above, or can be a fragment or variant thereof. Preferably, however, the mammal is a human. Another preferred mammal is canine (e.g. dog). Sequences of CD23 from various species are known in the art and thus appropriate CD23 molecules for use in the invention can be readily generated or produced by standard techniques, e.g. recombinant techniques. A fragment of the canine (e.g. dog) CD23 sequence is provided elsewhere herein (SEQ ID NO:32) and thus preferred constructs of the invention comprise this sequence or a sequence substantially homologous thereto, e.g. a sequence with at least 70%, 75%, 80% etc., identity thereto as described elsewhere herein.

Preferred target antigen for the CD23 (in this case IgE, in particular the IgE-Fc domain) is IgE from the same species or source as the species or source from which the chosen CD23 molecule is obtained or derived or corresponds to. Thus, where the chosen CD23 is human then preferably the target IgE is human IgE. In some embodiments however, IgE from other types of mammals, examples of which are described elsewhere herein, can also be used as target IgE, for example IgE protein from non-human primates such as cynomolgus monkeys are particularly preferred. Also preferred is canine (e.g. dog) IgE. In some embodiments it is desired that CD23 molecules (or fragments or variants) used in constructs show species cross reactivity in binding to target antigen (IgE). For example, the CD23 molecules (or fragments or variants) can specifically bind to both human and non-human primate forms of IgE or to both human and rodent (e.g. mouse or rat) or other non-human mammalian forms of IgE. In some embodiments the binding affinity of CD23 molecules (or fragments or variants) to the different species of target IgE, or the ability of CD23 molecules (or fragments or variants) to perform in functional assays using different species of target IgE, is preferably not substantially different from each other, e.g. is within 5 fold or 10 fold of each other. In particular the binding affinity (or functional activity) for human IgE is preferably not substantially different from, e.g. is within 5 fold or 10 fold of, the binding affinity (or functional activity) to the IgE from another mammalian species, e.g. non-human primate or rodent.

Part b) of the protein constructs of the invention can comprise any entity or molecule which can bind or enable binding to or target the neonatal Fc receptor (FcRn). Such binding to FcRn can be direct, i.e. with no intermediate, or indirect, e.g. via an intermediate entity. This direct or indirect binding to the FcRn can then enable recycling through the endosome providing that the binding is sensitive to endosomal conditions, e.g binding is observed or takes place in the endosomes but not outside the cells or in the extracellular environment, such as in serum or tissues. Thus, such endosomal sensitive binding of part b) is important for the preferred constructs of the invention.

Preferably and conveniently such an interaction or such binding would be direct. In other words, the entity or molecule making up part b) of the constructs can bind or interact directly with FcRn, for example can comprise any protein, peptide or polypeptide which can bind (e.g. specifically bind) to FcRn. Examples of such molecules are known in the art and any of these can be used. For example, molecules (e.g. binding proteins or peptides) which can bind directly to FcRn include albumin from various species, e.g. human serum albumin (HSA). In addition, appropriate Fc regions of antibodies, in particular IgG-Fc regions can also bind directly to FcRn. Thus, albumin, e.g. HSA, or fragments thereof or variants thereof (such as modified albumin molecules, e.g. modified HSA molecules) which bind to FcRn, or IgG-Fc regions, e.g. mouse or human IgG-Fc regions, preferably human IgG-Fc regions, or fragments thereof or variants thereof (such as modified IgG-Fc regions, e.g. modified human IgG-Fc regions) which bind to FcRn, are conveniently used in the constructs of the invention. As described elsewhere herein, other appropriate molecules which can bind or interact directly with FcRn are FcRn antibodies or other FcRn binding proteins or peptides. Such molecules are also referred to herein as FcRn binding proteins or entities.

The sequences of such FcRn binding proteins, such as albumins and IgG-Fc regions, including modified versions thereof which show retained or improved binding to FcRn are well known and described in the art and any of these can be used providing they have the ability to bind to FcRn and, in the context of the present invention, preferably enable recycling of the construct back to the extracellular environment, e.g. the serum or tissues.

IgG-Fc regions or fragments or variants thereof are especially preferred. IgG-Fc regions for use in the present invention are thus conveniently derived from or correspond to Fc regions as present in IgG molecules.

The term Fc region (or Fc fragment) as used herein has its art recognised meaning and comprises or corresponds to the part of an antibody that has the ability to interact with Fc receptors. Generally said Fc regions (or Fc fragments) are made up of two identical chains (are dimers) which comprise the CH2 and CH3 domains of an antibody.

In IgG, IgA and IgD antibody isotypes, the Fc regions (or Fc fragments) are made up of two identical chains (are dimers) which each comprise two heavy chain constant domains (CH2 and CH3) in each polypeptide chain. In IgM and IgE antibody isotypes, the Fc regions (or Fc fragments) are made up of two identical chains (are dimers) which comprise three heavy chain constant domains (CH2, CH3 and CH4) in each polypeptide chain. Within the IgG-Fc the two CH3 domains bind each other tightly, whereas the two CH2 domains have no direct protein-protein contact with one another. It is the tight binding of the CH3 domains that allows the dimers to form. Thus, in the constructs of the invention which comprise an IgG-Fc region (or a fragment or variant thereof), preferably the two identical chains of said region can bind to each other via the CH3 domains, thereby providing a linkage between the two polypeptide chains each containing one chain (or half) of the Fc region, thereby allowing the formation of a dimer. If other types of Fc region are used, then equally the parts of the Fc region which allow dimerisation are preferably included.

Truncated, mutated or modified Fc regions (or Fc fragments), e.g. fragment or variant Fc regions, in particular IgG-Fc regions, may be used provided that the ability to interact with the FcRn is maintained or present, or improved (e.g high or higher affinity binding), e.g compared to the starting, non-mutated or wild-type Fc region. Appropriate mutants with improved binding are well known and described in the art, e.g. YTE or LS mutants, and any of these may be used. Appropriate mutants with (or which confer) improved or increased half-life are well known and described in the art, and any of these may be used (see for example those mutants described in Wang and Brezski, 2018, Protein Cell 9 (1): 63-73, e.g. Table 1).

The IgG-Fc regions used in the constructs of the present invention can be derived from any subtype of IgG antibody, for example IgG1, IgG2, IgG3 or IgG4. In some embodiments, IgG1, IgG2, or IgG3 Fc regions are used, most preferably IgG1. In other embodiments an IgG4 Fc region is not used. In some embodiments, the Fc region may be engineered, or modified, to include additional or modified properties, e.g. additional or modified effector functions that may include the induction of an antibody-dependent cellular cytotoxicity (ADCC) or antibody dependent cellular phagocytosis (ADCP) response, complement-dependent cytotoxicity (CDC), or an increased half-life or increased co-engagement of antigen and/or Fcγ receptors. Modifications which reduce effector function can also be used, e.g. aglycosylated or afucosylated forms, or forms which show reduced Fcγ receptor binding (Fc silencing) and/or reduced C1q binding. Appropriate mutants with (or which confer) these features are well known and described in the art, and any of these may be used (see for example those mutants described in Wang and Brezski, 2018, Protein Cell 9 (1): 63-73, e.g. Table 1).

As mentioned above, appropriate Fc regions (IgG-Fc regions) for use in the constructs of the present invention comprise the CH2 and CH3 domains. In some embodiments, CH4 and/or CH1 domains can be included. However in other embodiments CH2 and CH3 domains (or fragments or variants thereof which have the ability to interact with the FcRn, and preferably the ability to dimerize) are the only parts of IgG antibodies included in the constructs. For example, in some embodiments no light chain antibody domains, in particular no light chain constant domains (CL domains) will be included in part b) of the constructs. In preferred embodiments the IgG-Fc regions are human IgG-Fc regions or canine (e.g. dog) IgG-Fc regions. Sequences of Fc regions and the positions of the CH1, CH2, CH3 and CH4 domains are readily available to the skilled person (see e.g. Wang and Brezski, 2018, supra). For example, exemplary human full IgG Fc sequences are provided in the sequence Tables elsewhere herein, from which exemplary sequences of CH1, CH2, CH3 and/or CH4 domains, as appropriate, preferably CH2 and CH3 domains, can be derived for inclusion in the constructs. In addition, an exemplary canine (e.g. dog) IgG Fc sequence (comprising the CH2 and CH3 domains) is provided elsewhere herein.

As the binding to FcRn can also be indirect, other types of molecule, for example molecules which themselves (or in turn) bind to FcRn binding proteins such as those described above, e.g. IgG Fc or albumin binding proteins, can also readily be used.

For example, part b) of the protein construct could mediate binding, e.g. specific binding, to a serum protein such as albumin, e.g. HSA, which in turn would bind to the FcRn, or would mediate binding, e.g. specific binding, to a circulating immunoglobulin molecule such as IgG. Thus, in embodiments where part b) of the protein construct can bind or specifically bind to albumin, e.g. HSA, or bind or specifically bind to IgG, then the constructs of the invention can also interact with or bind to the FcRn via HSA or IgG. Put another way, part b) of the protein construct binds to or targets a FcRn binding partner, or an agent that interacts with the FcRn receptor. Equally part b) of the protein construct could contain an antibody fragment (e.g. a Fab or other fragments such as sdAbs as described elsewhere herein) or other FcRn binding proteins (or peptides), e.g. binding proteins or single domain binding proteins as described elsewhere herein, which could specifically bind directly to the FcRn. Preferred molecules would be antibodies or antibody-based molecules, such as those containing antibody CDRs (e.g. 1 to 6 antibody CDRs) grafted onto an alternative scaffold. Particularly preferred molecules would be antibodies or antibody fragments (e.g. with 1 to 6 antibody CDRs). Preferred antibody fragments would be Fab fragments or single domain antibodies (sdAbs). In some embodiments sdAbs are used. In some embodiments Fab fragments are not used.

Thus entities, e.g. proteinaceous entities such as polypeptides, peptides, peptidomimetics, that bind to IgG, and recruit IgG-Fc to the construct in order to thereby in turn bind to FcRn can be used. There are many examples of different types of entities, e.g. proteinaceous entities, that can be used for this purpose and which can bind to IgG. Preferred molecules would be antibodies to IgG or antibody-based molecules, such as those containing antibody CDRs (e.g. 1 to 6 antibody CDRs) grafted onto an alternative scaffold. Particularly preferred molecules would be antibodies or antibody fragments (e.g. with 1 to 6 antibody CDRs). Preferred antibody fragments would be Fab fragments or single domain antibodies (sdAbs). In some embodiments sdAbs are used. In some embodiments Fab fragments are not used.

Equally entities, e.g. proteinaceous entities such as polypeptides, peptides, peptidomimetics, can be used which can bind to albumin and thereby recruit albumin, e.g. human serum albumin, to the construct in order to thereby in turn bind to FcRn. Any albumin binding proteins can be used for this purpose. Preferred binding proteins would be antibodies or antibody-based molecules, such as those containing antibody CDRs (e.g. 1 to 6 antibody CDRs) grafted onto an alternative scaffold. Particularly preferred molecules would be antibodies or antibody fragments (e.g. with 1 to 6 antibody CDRs). Preferred anti-albumin antibody fragments would be Fab fragments or single domain antibodies (sdAbs). In some embodiments sdAbs are used. In some embodiments Fab fragments are not used.

Albumin, e.g. HSA, or an albumin fragment or variant which can bind to FcRn, is a particularly preferred molecule to use in the constructs of the present invention (either directly or indirectly) as, like IgG-Fc, it shows pH-dependent binding to FcRn with no or low affinity binding at physiological pH (around pH 7.4) as found in the cytoplasm of the cell or in serum or in tissue (e.g. interstitial tissue) and good or high or higher affinity binding to FcRn at acidic or lower pH (e.g. endosomal pH, e.g. pH 6.5 or lower, e.g. pH 5.0 to 6.5, or a pH of around pH 6.0 or lower, e.g. pH 5.0 to 6.0, e.g. pH 6.0 or pH 6.5) to enable recycling of the construct containing albumin back to the serum from the endosomal compartment within cells.

Equally, any other entity which displays pH-dependent or endosomal-dependent FcRn binding such as that described for albumin or IgG-Fc can be used. Advantageously this pH-dependent or endosomal-dependent binding should allow recovery of the protein construct (or biotherapeutic or biologic) via the recycling pathway. Thus, part b) of the protein construct preferably provides the ability of the protein construct to be recycled, by providing an interaction to FcRn which is stable under endosomal conditions such as low pH and/or low calcium as described elsewhere herein, and less stable or absent under physiological or serum conditions such as pH 7.4 and/or high calcium as described elsewhere herein.

In some protein constructs of the invention said binding of part b) of the construct to FcRn is increased at endosomal calcium levels compared to serum calcium levels, or is increased at endosomal pH levels (e.g. at pH 6.0 or 6.5, or other endosomal pH levels as described elsewhere herein) compared to serum pH levels (e.g. pH 7.4). In some protein constructs of the invention said binding of part b) of the construct to FcRn is increased at pH 6.0 or 6.5 compared to pH 7.4.

Convenient and preferred examples of IgG or albumin binding proteins (or FcRn binding proteins) for use in the constructs of the present invention are single domain binding proteins. The term “single domain binding protein” as used herein (sometimes abbreviated to sdbp) is a monomeric protein which has a single protein domain which can, individually, mediate binding to a specific target antigen (e.g. albumin or IgG or FcRn), or is a single protein unit which is sufficient for a specific interaction with a target antigen. In other words, the single domain binding protein can alone specifically bind to a target antigen (e.g. albumin or IgG or FcRn). Single domain binding proteins for use in the present invention are thus proteins with a single protein domain but which contain an antigen binding site (e.g. a binding site for albumin or IgG or FcRn). In other words, the antigen binding site of a sdbp is formed only by a single domain. Any appropriate antigen binding site can be present. For example, such single domain binding proteins will often contain one or more complementarity determining regions (CDRs) to mediate antigen binding. Three CDR regions may be present, although it is possible for antigen binding to be mediated by even one or two CDR regions, especially if only a low or moderate affinity binding is desired. Thus sdbps containing one or two CDRs are also included. Appropriate sdbps can be naturally produced or derived from natural sources, or can be in the form of engineered or recombinant molecules/binding proteins.

Preferred single domain binding proteins for use in the constructs of the invention are single domain antibodies (sdAbs) which are also referred to herein and in the art as nanobodies or V_{H}H antibodies, or VH antibodies or VL antibodies. Such single domain antibodies only comprise a single variable antibody domain, but, like a whole antibody, are able to bind selectively to a specific antigen (e.g. albumin or IgG or FcRn). Because such single domain antibodies consist of a single monomeric variable antibody domain, they are much smaller than conventional antibodies and are also smaller than Fab or single chain variable fragments (scFvs) or Fvs.

Any sdbp which is capable of specifically binding individually to an epitope on a target antigen (e.g. albumin or IgG or FcRn) can however be used in the constructs of the present invention. For example, as well as immunoglobulin based sdbps which generally comprise CDR regions (and optionally FR regions or an immunoglobulin based scaffold), in some embodiments non-immunoglobulin based single domain binding proteins/scaffold proteins can be used which can be selected for the ability to specifically bind to a particular target antigen (e.g. albumin or IgG or FcRn) in their own right. Such molecules are also referred to as antibody mimics (or antibody mimetics). Examples of appropriate non-immunoglobulin based single domain binding proteins are known and described in the art and include fibronectins (or fibronectin-based molecules), for example based on the tenth module of the fibronectin type III domain, such as Adnectins (e.g. from Compound Therapeutics, Inc., Waltham, Mass.); affimers (e.g. from Avacta); ankyrin repeat proteins (e.g. from Molecular Partners AG, Zurich, Switzerland); lipocalins, e.g. anticalins (e.g. from Pieris Proteolab AG, Freising, Germany); human A-domains (e.g. Avimers); staphylococcal Protein A (e.g. from Affibody AG, Sweden); thioredoxins; and gamma-B-crystallin or ubiquitin based molecules, e.g. affilins (e.g. from Scil Proteins GmbH, Halle, Germany). Such molecules can also be used as scaffolds onto which appropriate CDRs which mediate target antigen binding can be grafted. For example, the CDRs of an appropriate immunoglobulin based sdbp, e.g. a sdAb, can be grafted onto an appropriate non-immunoglobulin scaffold.

Although preferred antibody fragments for use in the constructs of the present invention are sdAbs, other antibody fragments comprising one or more, two or more, or three or more CDRs, e.g. antibody fragments with 6 CDRs, can equally be used, such as scFvs, Fabs or Fab-like molecules. In some embodiments Fabs or Fab-like molecules are not used.

The FcRn binding entities, e.g. albumin or IgG-Fc (or other Fc) or binding proteins (or peptides) for albumin or IgG or FcRn, for use in the constructs of the present invention can be obtained from or be derived from any appropriate source or species, or can correspond to FcRn binding entities from such sources or can be a fragment or variant thereof. Preferred sources are mammalian, and any appropriate mammalian source may be used, for example humans or any livestock, domestic or laboratory animal. Specific examples include mice, rats, pigs, cats, dogs, sheep, rabbits, horses, cows and non-human primates (e.g. monkey, e.g. cynomolgus monkey). Preferably, however, the mammal is a human and the sequences are, or correspond to, human sequences or human derived sequences. Other preferred mammals are canine (e.g. dog). Sequences of FcRn binding entities, e.g. albumin or IgG-Fc (or other Fc), from various species are known in the art and thus such FcRn binding entities can be readily generated or produced by standard techniques, e.g. recombinant techniques. For example an exemplary sequence of a canine IgG-Fc sequence comprising the CH2 and CH3 domains is provided below (SEQ ID NO:33) and thus preferred constructs of the invention (for example for use in canines) comprise this sequence, or any other sequence comprising the canine CH2 and CH3 domains, or a sequence substantially homologous thereto, e.g. a sequence with at least 70%, 75%, 80% etc., identity thereto as described elsewhere herein.

KTKVDKPVPKRENGRVPRPPDCPKCPAPEMLGGPSVFIFPPKPKDTLLIARTPEVTCV
VVDLDPEDPEVQISWFVDGKQMQTAKTQPREEQFNGTYRVVSVLPIGHQDWLKGKQ
FTCKVNNKALPSPIERTISKARGQAHQPSVYVLPPSREELSKNTVSLTCLIKDFFPPDI
DVEWQSNGQQEPESKYRTTPPQLDEDGSYFLYSKLSVDKSRWQRGDTFICAVMHE
ALHNHYTQESLSHSPGK
(Canine IgG Fc, SEQ ID NO: 33, derived from Genbank AAL 35302).

Equally, the FcRn binding entities, e.g. albumin or IgG-Fc or binding proteins for albumin or IgG, for use in the constructs of the present invention can be synthetic or recombinant molecules, for example molecules identified by library screening.

Preferred FcRn target for the FcRn binding entities in part b) of the protein construct can be any desired species, for example is from the same species or source as the species or source from which the chosen FcRn binding entity is obtained or derived or corresponds to. Thus, where the FcRn binding entity is human then preferably the FcRn target is human FcRn. Where for example the FcRn binding entity is canine (e.g. dog) then preferably the FcRn target is canine (e.g. dog) FcRn. The appropriate species of FcRn target will also generally depend on the species (e.g. mammalian species) to which the protein construct of the invention is to be administered. For example, where the administration is to humans then part b) of the construct should be able to bind to human FcRn, etc., depending on the species.

In some embodiments however, as described above for parts a) of the constructs, the FcRn binding entities can cross react with FcRn proteins from other types of mammals (i.e. show species cross reactivity), examples of which are described elsewhere herein, for example cross reactivity with FcRn from non-human primates such as cynomolgus monkeys is particularly preferred. For example, the FcRn binding entities can specifically bind to both human and non-human primate forms of FcRn or to both human and rodent (e.g. mouse or rat) forms of FcRn. In some embodiments the binding affinity of the FcRn binding entities to the different species of target FcRn, or the ability of FcRn binding entities to perform in functional assays using different species of target FcRn, is preferably not substantially different from each other, e.g. is within 5 fold or 10 fold of each other. In particular the binding affinity (or functional activity) for human FcRn is preferably not substantially different from, e.g. is within 5 fold or 10 fold of, the binding affinity (or functional activity) to the FcRn from another mammalian species, e.g. non-human primate or rodent.

The protein constructs of the present invention can be manufactured or generated in any appropriate way. For example, the various components of the constructs can be encoded on a single polypeptide chain or multiple polypeptide chains after which the various components are then joined or linked together.

The various components of the protein constructs of the invention can be attached to or linked to each other in any appropriate way such that each part can carry out their function. In some embodiments of the invention the protein constructs will contain linkers (physical linkers or linker molecules, e.g. at least one physical linker or linker molecule) between different parts of the construct, e.g. between part(s) a) and part(s) b) of the constructs. For example, said linker can be used to join a CD23 molecule (or CTLD) of part a) of the construct to an FcRn binding entity of part b), for example, in preferred embodiments, join a CD23 (or CTLD) monomer to one of the polypeptide chains making up an Fc region, e.g an IgG-Fc region. Any appropriate linker molecules can be used which would be well known to a person skilled in the art. For example, peptide linkers or chemical linkers or other covalent linkers can be used as appropriate.

Peptide (protein) linkers are generally preferred. Such peptide linkers, which may comprise non-natural or natural amino acids, are well known in the art, and appropriate linkers with an appropriate sequence, length, and/or flexibility/rigidity, can thus readily be selected by a skilled person in order to allow the various components of the protein constructs of the invention to be attached together in a stable way but with the correct spatial orientation or spatial optimisation so that the above required functional properties (i.e. the functional properties of each component) are retained once the individual components are attached to each other. For example, in the case of a pair of (two) CD23 molecules (or fragments or variants), for example as shown in the schematic of FIG. 1, both members of the pair (e.g. both monomers) preferably need to be in appropriate proximity to be able to bind a single molecule of target IgE (one CD23 molecule binding to each chain of the IgE Fc dimer, which is also referred to herein as cis-binding) as opposed to for example two molecules of target IgE (sometimes referred to herein as trans-binding), and peptide linkers, or other means of attachment, can be designed appropriately.

However, in addition, or alternatively, constructs with such linkers, or other appropriate linkers, can also link for example two molecules of target IgE (e.g. via two CD23 molecules, preferably monomers, one CD23 molecule binding to each individual IgE molecule). Such linkages can also be referred to as trans-binding linkages where one preferred protein construct of the invention (e.g. comprising at least two CD23 molecules/monomers) is linked to two IgE target molecules via the two CD23 molecules (monomers). Higher order structures, e.g. as described elsewhere herein, can then be formed, for example involving multiple protein constructs of the invention and multiple molecules of target IgE. It is believed that the most stable structures or higher order structures (and therefore preferred in some embodiments) are those in which all the CD23 binding sites on the IgE molecules are occupied as described elsewhere herein. Thus, linkers which allow such conformations and structures to be formed are appropriate.

Although the constructs of the invention do not necessarily have to be provided in the form of a fusion protein, where for example the polypeptides making up part(s) a) and part(s) b) are linked to form a genetic fusion, genetic fusions or constructs in which parts a) and b) are linked together in a single polypeptide or protein chain are preferred. For this reason, preferred linkers are peptide or protein (polypeptide) linkers. Any appropriate peptide linker may be used providing that the linker does not interfere with the function of part a) or part b) of the construct, or indeed any other part of the construct.

Thus, the linker or spacer can aid the folding of the connected proteins, and the spacer or linker length and/or flexibility/rigidity, can be adjusted as appropriate to enable the best or satisfactory functional folding of each component. Appropriate lengths could readily be determined by a person skilled in the art and could be any appropriate number of amino acids. However exemplary lengths might be at least 5, 10, 15, 20, 25, 30, 35, 40, or 45 amino acids long (e.g. at least 6, 7, 8, or 9 amino acids long, or at least 11, 12, 13, or 14 amino acids long), or be between 5 or 10 and 50 amino acids, e.g. 5 or 10 to 15, 20, 25, 30, 35, 40, 45 or 50 amino acids, or 15 to 20, 25, 30, 35, 40, 45 or 50 amino acids, or 20 to 25, 30, 35, 40, 45 or 50 amino acids, or 25 to 30, 35, 40, 45 or 50 amino acids, or 30 to 35, 40, 45 or 50 amino acids. Preferred linkers can be 15 to 30 amino acids long, e.g. can be or be up to 15, 20, 25 or 30 amino acids long (or up to 40 or 50 amino acids long). Exemplary linkers are described in the art and can include GS linkers such as one or more repeats of the G4S linker (GGGGS, SEQ ID NO:16). The linker used in the constructs used in the attached Examples has the sequence GGGGSGGGGSGGGGS (SEQ ID NO: 17), i.e. 3 repeats of GGGGS. Linkers with 4 repeats are also used. This linker is thus preferred for some embodiments of the invention, e.g. when the protein constructs have or are capable of forming the construction such as that shown in FIG. 1, but it will be appreciated that linkers (spacers) with other sequences and lengths, e.g. other GS linkers, or other appropriate linkers with the same, similar, or equivalent lengths can also be used. For example, GGGGS linkers with 4 or 6 repeats have also been shown to be effective and are preferred. Equally linkers with 5, or 7, or more repeats can be used providing the relevant functional properties of the constructs are retained.

Indeed, the presence of linkers, for example peptide linkers, has been shown to provide advantages to the constructs of the invention in terms of functionality. Thus, as can be seen from the data in the Examples, the presence of linkers between parts a) and b) of the construct, e.g. between the CD23 comprising part and the FcRn binding entity (e.g. a polypeptide chain making up an Fc region), has been shown to result in improved (or increased) ability to inhibit the activity of target antigen, IgE, compared to constructs where no linkers were present. Specifically, an improved (or increased) ability to inhibit the binding of IgE to the high affinity receptor, FcεRI, compared to constructs where no linkers were present. In addition, the longest linker length tested (here 20 amino acids) showed the best functional properties. Thus, appropriate linkers for use in the present invention could be any linker which resulted in improved (or increased) ability to inhibit the activity of target antigen, IgE, compared to constructs where no linkers (or short linkers for example with fewer than five amino acids) were present. Specifically, any linker which resulted in an improved (or increased) ability to inhibit the binding of IgE to the high affinity receptor, FcεRI, compared to constructs where no linkers (or short linkers for example with fewer than five amino acids) were present would be appropriate.

Selection of the nature of the linker and other properties such as appropriate linker lengths, for example to achieve the same (or similar) effects to those observed for the linkers used in the exemplified constructs, would be a standard and routine procedure for a person skilled in the art. For example, in certain embodiments of the invention, for example where protein constructs with or capable of forming the structure as shown in FIG. 1 are concerned (e.g. forming a structure with cis-interactions as described herein), an appropriate linker length and/or structure/nature can be selected which allows the two (or pair of) CD23 molecules to bind to each chain of the same IgE Fc, thereby preferably allowing improved overall binding affinity by virtue of an avidity effect. Similar selections can take place for linkers in protein constructs capable of linking different IgE molecules and forming a structure with trans-interactions as described herein, in particular higher order structures or oligomers as described herein, thereby also preferably allowing improved overall binding affinity by virtue of an avidity effect. Other features for appropriate linkers which could be selected would be well-known to a person skilled in the art and could be readily selected. For example, preferably the linkers will not be antigenic or contain protease cleavage sites.

An advantage with using peptide linkers is that it enables production as a single polypeptide (as discussed above). However, any other appropriate means of attachment might also be used, for example any other form of linker, including chemical linkers, providing that the functional properties (as discussed elsewhere herein) of the various components which are linked together are retained once the components are attached to each other.

Thus, the molecules of the invention can be regarded as comprising two main molecular components or modules. Generally, parts a) and b) of the construct are separate components which are attached or linked to each other by any appropriate means to retain functionality of all components. In embodiments where a single polypeptide chain is provided and a single molecule of CD23 (or fragments or variants) is present then the CD23 molecule (or fragment or variant) can be placed at the N-terminus or the C-terminus of the chain, more preferably at the N-terminus. The FcRn binding entity can then conveniently be placed at the other end of the polypeptide chain, preferably at the C-terminus. Linkers can be included as described elsewhere herein. Alternative configurations can of course be conceived providing that all the components retain their biological function.

In embodiments where a single polypeptide chain is provided and two molecules of CD23 (or fragments or variants) are present then the CD23 molecules (or fragment or variant) can be placed at both the N-terminus and the C-terminus of the chain (one at each end), with the FcRn binding entity in between. Linkers can be included as described elsewhere herein. Exemplary FcRn binding entities are described elsewhere herein but preferred in these aspects might be single chain (e.g. single domain) antibodies or single chain (e.g. single domain) binding proteins. Other preferred FcRn binding entities could be albumin molecules as described elsewhere herein. Thus, an exemplary construct might comprise two molecules of CD23 (or fragments or variants) and albumin, or two molecules of CD23 (or fragments or variants) and a single domain antibody (or other single domain binding protein) specific for FcRn. Optionally and preferably linkers can be present, for example between the CD23 molecules and the FcRn binding entities.

Alternative configurations can of course be conceived providing that all the components retain their biological function. Thus, in such single chain embodiments with two molecules of CD23 (or fragments or variants), the two molecules of CD23 (or fragments or variants) can be present together (in tandem or adjacent to each other), at either the N-terminal or the C-terminal half (or end) of the chain and the FcRn binding entity can then conveniently be placed in the other half (or end) of the polypeptide chain. Exemplary components are as described above and elsewhere herein. Thus, an exemplary construct might comprise two molecules of CD23 (or fragments or variants) adjacent to each other and albumin, or two molecules of CD23 (or fragments or variants) adjacent to each other and a single domain antibody (or other single domain binding protein) specific for FcRn. Optionally and preferably linkers can be present, for example between the two individual CD23 molecules and/or to join the two CD23 molecules (which are present together) to the FcRn binding entities.

Conveniently in embodiments of the invention where the protein constructs comprise two polypeptide chains, the CD23 molecules (or fragments or variants) can be placed at the N-terminus or the C-terminus of each of the two chains, more preferably at the N-terminus. The FcRn binding entities can then conveniently be placed at the other end of the polypeptide chains. Constructs with two polypeptide chains are particularly preferred in embodiments where the entity which can bind to FcRn (i.e. part b) of the constructs) is itself made up of two polypeptide chains, e.g. is an IgG-Fc domain. An exemplary structure of such a construct is shown in FIG. 1, where the first chain (one chain) of the construct comprises CD23 (a CTLD of CD23) and one chain of the IgG-Fc and the second chain (other chain) of the construct comprises a second CD23 (a CTLD of CD23) and the other chain of the IgG-Fc. The CD23 molecules are connected to the individual chains of the IgG-Fc by appropriate linkers and the two polypeptide chains are linked together via the natural association of the two chains of the IgG-Fc via the CH3 domains. In preferred constructs, the CD23-based molecules can be placed at the N terminus via a linker to the FcRn binding entity at the C terminus. Similar structures can be used and are preferred for other part a) and part b) molecules as described elsewhere herein. Equally, some constructs can contain four individual CD23 molecules and conveniently, in said constructs, the CD23 molecules can be placed at the N terminus and C terminus of both polypeptide chains, with the FcRn binding entity in between the two CD23 molecules on each chain. Equally, some constructs can contain four, eight, or multiples of four, copies of individual CD23 molecules arranged linearly, and conveniently, in said constructs, the CD23 molecules can be placed at the N terminus and C terminus of both polypeptide chains, with the FcRn binding entity in between the CD23 molecules on each chain. Linkers are preferably used to connect each of the two molecules of CD23 to the FcRn binding entity on each chain.

The term “fusion protein”, “fused”, etc., is used herein to describe the functional joining of two or more protein components in the same polypeptide sequence or in the same open reading frame (ORF). An example of such fusion proteins can also be described as genetic fusions as they are encoded by the same nucleic acid sequence (sometimes called a “fusion gene” or “fusion nucleotide sequence”). Although two (or more) protein components (or encoding nucleic acid sequences) can be directly adjacent to each other in such a fusion protein, equally and preferably the components can be joined by appropriate peptide spacers or linkers. As is well known in the art, spacers or linkers can be important to allow each of the individual protein components to be expressed in a functional manner, e.g. allowing them to form the appropriate three-dimensional structure to perform or maintain their native or desired function.

Thus, in the fusion proteins present in the protein constructs of the invention, a peptide spacer (or linker) is generally included between a CD23 molecule (or fragment or variant), e.g. a molecule comprising a CTLD of CD23 (or fragment or variant), i.e. part a) of the construct and a FcRn binding entity, i.e. part b) of the construct. Thus, such constructs can contain at least one linker. Generally, in embodiments where two (or more) molecules (or monomers) comprising soluble CD23 (or a CTLD of CD23) are included then two (or more), or at least two, linkers are included, such that for example each monomer (or molecule) of CD23 is separately linked (via a linker) to the entity which can bind to FcRn (part b) of the construct), although equally, in embodiments where two or more CD23 monomers/molecules are present together, e.g in tandem (or consecutively), in a construct, then only one of the monomers (or molecules) of CD23 might be linked (via a linker) to the entity which can bind to FcRn (part b) of the construct). Thus, preferably each monomer of CD23 has a linker. For example, when two, four or six etc., CD23 monomers/molecules are present in the constructs then two, four or six etc., respectively, separate linkers can be present (one for each CD23 monomer/molecule). In other embodiments, such linkers or spacers need not be included, or may only be included in between some of the components. The presence of such linkers is however preferred as it has been shown that this results in significant improvement in functionality of the constructs.

Although this discussion focuses on a linker or spacer between parts a) and parts b) of the constructs, linker sequences may be included elsewhere in the constructs of the invention as appropriate, e.g. between other components of the constructs which may be present.

The various components of parts a) and b) of the protein constructs of the invention as described herein can be produced or selected using methods which are standard in the art and then linked or attached together in any appropriate manner such that all the components retain their functional properties as described herein. Thus, in preferred embodiments, single or multiple copies of CD23 (or fragments or variants), e.g. single or multiple copies of a molecule comprising a CTLD of CD23 (or fragment or variant), which recognise one or more molecules of IgE, are attached or linked to one or more entity which can bind to FcRn to form a protein construct which will spatially orientate the sCD23 molecules (or fragments or variants) such that they can bind IgE and the FcRn binding entities such that they can bind FcRn.

In embodiments where single domain binding proteins (sdbps) which can bind to target antigen (e.g. IgG, albumin or FcRn) are used, these can be derived appropriately by methods well known and described in the art. For example, sdAbs for use in the methods of the present invention can be produced by methods which are well known and standard in the art, for example by immunizing camelids such as dromedaries, camels, llama or alpaca, or other species such as rats or mice (e.g. in the form of transgenic animals capable of expressing fully functional human heavy chain antibodies), with the desired antigen and then screening for sdAbs by appropriate methods, e.g. by preparing and screening gene libraries, e.g. phage display libraries, from the lymphocytes of the immunized animals, for antigen specific binders with desired affinity for the target antigen.

Alternatively, sdAbs can be generated or identified by screening naïve gene libraries prepared from appropriate animals which have not been immunized. Alternatively, sdAbs can be made from conventional antibodies or antibody fragments or synthetic libraries by screening for single VH or VL domains which can bind target antigen.

In embodiments where the sdbps with specificity for target antigen (e.g. IgG, albumin or FcRn) are or comprise fibronectins (e.g. adnectins), affimers, ankyrin repeat proteins, lipocalins (e.g. anticalins), human A-domains (e.g. Avimers), staphylococcal Protein A, thioredoxins and gamma-B-crystallin or ubiquitin based molecules, e.g. affilins, again these can be generated or selected using art described methods.

Individual sdbps (e.g. sdAbs) which bind to target antigen (e.g. IgG, albumin or FcRn) with low, moderate or high affinity (as desired) can readily be selected, for example by carrying out screening under appropriate stringency conditions.

In embodiments of the invention where the binding of the various components to target antigen, e.g. sdbps (e.g. sdAbs, or other antibody molecules or antibody fragments) are pH sensitive (or endosomal sensitive), components or molecules selected as described above for individual low, moderate or high affinity binding to target antigen are then tested for pH sensitive (e.g. endosomal pH sensitive) binding by testing their ability to bind target antigen at acidic pH, e.g. pH 6.5 or pH 6.0 (or a lower selected pH) and selecting molecules which have good or high affinity binding at acidic pH, e.g. pH 6.5 or pH 6.0, etc., but reduced binding at neutral pH, e.g. pH 7.4. Similar selections can be done under conditions of low (endosomal) calcium and high (physiological) calcium concentrations as described elsewhere herein, to select molecules which show calcium dependent (or other type of endosomal-dependent) binding.

Exemplary high affinity binders might have a Kd of <1 nM, moderate affinity binders might have a Kd≥1 nM to <50 nM and low affinity binders might have a Kd of 50 nM.

Alternatively, one or more of the individual components or molecules, e.g. sdbps (e.g. sdAbs or other antibody molecules or antibody fragments) can be subjected to protein engineering, e.g. by modifying or mutating CDR or other amino acid residues, before testing as described above in order to produce individual molecules which show pH sensitive, or other types of endosomal-sensitive, binding. A preferred method to produce pH sensitive molecules is to subject the CDRs or other amino acid residues to histidine engineering as is well known and described in the art.

In embodiments where the protein construct of the invention comprises an antigen binding fragment (antibody fragment) such as a Fab fragment, these can be derived appropriately by methods well known and described in the art, for example by immunization of animals with the target entity of interest, followed by the preparation and screening of appropriate libraries of the antibody fragments for fragments which bind to the appropriate target entity, e.g. HSA, IgG or FcRn. Alternatively, existing libraries can be screened for an appropriate antibody fragment which binds to a target entity, or an already known or described antibody fragment which binds to the chosen target entity can be used. Alternatively, appropriate antibody fragments can be made from conventional whole antibodies which can bind to the appropriate target entity and subjecting them to appropriate protein engineering or cleavage.

If necessary, one or more of the components of the protein constructs of the invention can be subjected to humanization before human clinical use or can be modified as appropriate to be compatible with administration to any non-human species to be treated. Again, appropriate methodologies and techniques to do this are well known and described in the art. Preferred components of the protein constructs of the invention for use in the protein constructs of the invention, for example used to bind target antigen, will be free of potential sites for post-translational modification, especially within the CDR regions or other binding sites. Potential sites for post-translational modification can be determined by art recognised and standard defined criteria.

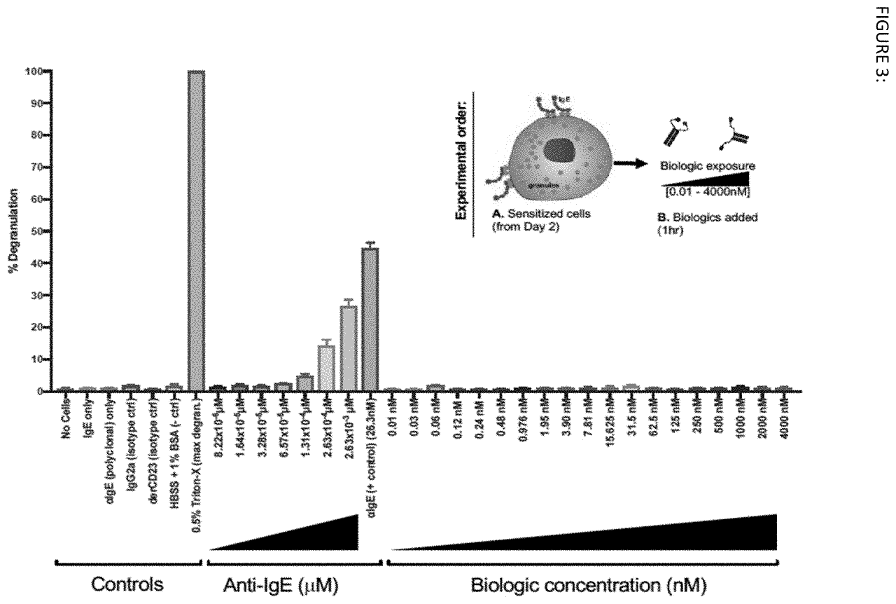

Whilst not wishing to be bound by theory, it is believed that when the constructs of the invention are used, one or more molecules of IgE is captured or binds to part a) of the protein constructs of the invention, e.g. binds to soluble CD23 or a fragment or variant thereof, e.g. a molecule comprising a CTLD of CD23 (or fragment or variant thereof). The complex between the protein construct and IgE (construct-IgE complex) is endocytosed or pinocytosed, taken up by endosomes and enters the endocytosis pathway. As the pH drops in the endosome, part b) of the protein construct of the invention binds to FcRn and is recycled to the cell surface (the construct is released or dissociates from FcRn at neutral pH, e.g. serum pH of around 7.4, and is therefore free to bind more IgE target) whilst the drop in calcium concentration (or pH) in the endosome causes release or dissociation of IgE from part a) of the construct, e.g. the CD23 based part, and IgE (unbound or free IgE) then enters the lysosomal degradation pathway and is destroyed. A schematic demonstrating the mode of action of the constructs of the invention is shown in FIG. 2.

Indeed, the experimental results herein show that extremely high efficiency of IgE uptake, e.g. up to 100% IgE uptake by cells with up to 98% retention or degradation of IgE with no measurable IgE recycling, is observed using the constructs of the invention. In addition, the recycling of the protein construct (biologic) to serum via FcRn binding is also shown to be extremely efficient, for example with up to 98% recovery of the biologic. This is in contrast to results observed for omalizumab where up to 100% IgE uptake by cells is also observed, but with up to 55% of the IgE being recycled.

Thus, preferred constructs of the present invention enable at least 60%, 65%, 70%, 75%, 80%, 85%, 90% or 95% of captured (or bound) IgE to be retained and/or degraded in cells after uptake. The ability of constructs of the invention to do this can be measured by any appropriate assay. Conveniently this can be assessed by way of an in vitro assay using appropriate cells, e.g. a recycling and cell uptake assay, for example as described in Example 3, or a simple IgE uptake or degradation assay. Preferred constructs of the present invention show recycling levels, in particular recycling of unloaded constructs, i.e. constructs no longer bound to IgE target, of at least 60%, 65%, 70%, 75%, 80%, 85%, 90% or 95% after cellular uptake. The ability of constructs of the invention to do this can be measured by any appropriate assay. Conveniently this can be assessed by way of an in vitro assay using appropriate cells, e.g. a recycling and cell uptake assay, for example as described in Example 3.

These two properties of highly efficient empty (unloaded) biologic recycling and IgE uptake/degradation/intracellular retention in combination can advantageously result in deeper IgE suppression, enhanced duration of action of the drug (increased half-life) and lower maintenance doses. The inventors thus believe that the constructs of the present invention provide a novel class of biotherapeutic (biologic) for targeting IgE.

The protein constructs of the invention can readily be manipulated to include other components or functions as desired. For example, a component can be included which can confer cell killing activity into the molecule, or the constructs can be modified or adapted so that for example ADCC, CDC or ADCP activity is present. In such cases, the constructs of the invention could be used to for example target membrane IgE expressed on the surface of cells via part a) of the construct, e.g. via the CD23 part of the construct, and the cells expressing membrane IgE on the surface could then be killed. Thus, this would allow direct targeting and killing of IgE expressing cells, e.g. B cells, e.g. B cells that have been stimulated to express membrane IgE/IgE expressing B cells, plasma blasts or plasma cells. It should be noted that there is a distinction between cells expressing membrane IgE, which would be targeted by the above approach, and cells which have surface IgE, e.g. because IgE has become bound to a cell surface receptor, e.g. FcεRI, which would preferably not be targeted. In this regard, cells expressing the membrane form of IgE contain a C-terminal extension not present on soluble IgE (or on IgE bound to for example FcεRI) comprising a short cytoplasmic tail and a transmembrane domain such that it is presented as part of the B cell receptor at the cell membrane of IgE expressing B cells only.

In all the embodiments of the invention the protein constructs are man-made constructs in that they do not correspond to molecules that occur naturally, although some of the individual components of the constructs may correspond to native proteins or molecules (or parts thereof). In other words the protein constructs of the invention are non-native. Such protein constructs may thus be viewed as recombinant constructs or engineered constructs, e.g. made by genetic or recombinant engineering techniques which are well known in the art.

Although the above discussion is focused on describing the protein constructs of the invention, conveniently said protein constructs will be prepared or produced using appropriate nucleic acid molecules encoding all or part of such protein constructs.

Thus, it can be seen that nucleic acid molecules, e.g. one or more nucleic acid molecules (e.g. a set of nucleic acid molecules), comprising nucleotide sequences that encode the protein constructs, preferably the recombinant protein constructs, of the present invention as defined herein, or parts (for example single chains or the first or second chains of the protein constructs) or fragments thereof, form yet further aspects of the invention. Expression vectors comprising such nucleic acid molecules, e.g. one or more nucleic acid molecules, and host cells comprising said expression vectors or nucleic acid molecules or protein constructs form yet further aspects.

Typically, the one or more nucleic acid fragments encoding the protein constructs of the invention are incorporated into one or more appropriate expression vectors in order to facilitate production of the protein constructs, e.g. the recombinant protein constructs, of the invention.

The invention therefore contemplates an expression vector, e.g. one or more expression vectors, e.g. one or more recombinant expression vectors, containing or comprising a nucleic acid molecule of the invention, and the necessary regulatory sequences for the transcription and translation of the protein sequence encoded by the nucleic acid molecule of the invention. The vectors may also contain sequences to enable antibiotic resistance and replication of the vector. Suitable vectors and regulatory sequences would be well known to a person skilled in the art.

Expression vectors, e.g. recombinant expression vectors, of the invention, or nucleic acid molecules of the invention, can be introduced into host cells to produce a transformed host cell. The terms “transformed with”, “transfected with”, “transformation” and “transfection” are intended to encompass introduction of nucleic acid (e.g. a vector) into a cell by one of many possible techniques known in the art. Suitable methods for transforming and transfecting host cells can be found in Sambrook et al., 1989 (Sambrook, Fritsch and Maniatis, Molecular Cloning: A Laboratory Manual, 2nd Ed., Cold Spring Harbor Press, Cold Spring Harbor, N.Y., 1989) and other laboratory textbooks.

Suitable host cells include a wide variety of eukaryotic host cells and prokaryotic cells. For example, the molecules of the invention may be expressed in yeast cells, or mammalian cells, or prokaryotic cells such as Escherichia coli or Pichia pastoris.

A yet further aspect of the invention provides a method of producing the protein constructs of the invention, comprising a step of culturing the host cells of the invention. Preferred methods comprise the steps of (i) culturing a host cell comprising one or more of the recombinant expression vectors or one or more of the nucleic acid sequences of the invention under conditions suitable for the expression of the encoded protein construct; and optionally (ii) isolating or obtaining the expressed protein construct from the host cell or from the growth medium/supernatant. Such methods of production may also comprise a step of purification of the protein product and/or formulating the protein product into a composition including at least one additional component, such as a pharmaceutically acceptable carrier or excipient.

As the preferred recombinant molecules/protein constructs of the invention are made up of two (or more) identical polypeptide chains (e.g. each chain contains a CD23 molecule connected by a peptide linker to a chain of the IgG-Fc), then, in such embodiments, a single appropriate polypeptide chain is expressed in the host cell, so that the complete protein constructs of the invention can assemble in the host cell and be isolated or purified therefrom.

The protein constructs of the invention can be produced, purified or isolated by standard methods which would be well known to a person skilled in the art. However, a yet further aspect of the invention comprises the use of an affinity matrix (for example an affinity column or other solid-phase) to which IgE Fc (or a fragment, e.g. functional fragment, or variant thereof) has been immobilised (and which can then be used to capture the CD23 molecules in the constructs) for such steps of purification or isolation. Such an affinity matrix can thus also be used in methods of manufacture or production of the protein construct.

In such methods, such production, purification or isolation can be carried out by contacting an affinity matrix (for example an affinity column or other solid-phase) to which IgE Fc has been immobilised with the constructs of the invention under conditions such that the constructs (in particular the CD23 molecules in the constructs) bind to the IgE Fc on the affinity matrix. Such conditions could conveniently and preferably be those corresponding to serum (or physiological) calcium or pH levels (e.g. calcium levels of 1 to 2 mM or a pH of at or about pH 7.4) as described elsewhere herein. Such a binding step can then be followed by an elution step (i.e. a step of eluting the constructs from the affinity matrix) under conditions such that the constructs (in particular the CD23 molecules in the constructs) no longer bind to the IgE Fc on the affinity matrix (or are released from the IgE Fc on the affinity matrix). Such conditions could conveniently and preferably be those corresponding to endosomal calcium or pH levels (e.g. calcium levels of 3-30 μM or a pH of at or about pH 5.0 to 6.5, e.g. a pH of 6.0 or 6.5) as described elsewhere herein. Such an elution step in turn enables the isolation, purification, production or manufacture of the protein construct of the invention.

Compositions comprising a protein construct of the invention (or nucleic acid molecules or expression vectors of the invention) constitute yet further aspects of the present invention. Formulations (compositions) comprising one or more protein constructs (or nucleic acids or expression vectors) of the invention in a mixture with a suitable diluent, carrier or excipient constitute a preferred embodiment of the present invention. Such formulations may be for pharmaceutical use (are pharmaceutical compositions) and thus compositions of the invention are preferably pharmaceutically acceptable. Suitable diluents, excipients and carriers are known to the skilled man.

The compositions according to the invention may be presented, for example, in a form suitable for oral, nasal, parenteral, intravenal, topical or rectal administration. Unless otherwise stated, administration is typically by a parenteral route, preferably by injection subcutaneously, intramuscularly, intracapsularly, intrathecally, intraperitoneally, intratumouraly, transdermally or intravenously. In some embodiments subcutaneous administration is preferred.

The protein constructs of the invention defined herein may be presented in the conventional pharmacological forms of administration, such as coated tablets, nasal or pulmonal sprays, solutions, liposomes, powders, capsules or sustained release forms. Conventional pharmaceutical excipients as well as the usual methods of production may be employed for the preparation of these forms.

Injection solutions may, for example, be produced in the conventional manner, such as by the addition of suitable preservation agents or stabilizers. The solutions are then filled into injection vials or ampoules.

Nasal sprays may be formulated similarly in aqueous solution and packed into spray containers, either with an aerosol propellant or provided with means for manual compression.

Parenteral administration may be performed by subcutaneous, intramuscular or intravenous injection by means of a syringe, optionally a pen-like syringe. Alternatively, parenteral administration can be performed by means of an infusion pump. A further option is a composition which may be a powder or a liquid for the administration of the molecule or protein construct in the form of a nasal or pulmonal spray. As a still further option, the molecules or protein constructs of the invention can also be administered transdermally, e.g. from a patch, optionally an iontophoretic patch, or transmucosally, e.g. bucally.

Suitable dosage units can be determined by a person skilled in the art.

The pharmaceutical compositions may additionally comprise further active ingredients in the context of co-administration regimens.

The protein constructs of the invention as defined herein may be used as molecular tools for in vitro or in vivo applications and assays. Thus, yet further aspects of the invention provide a reagent that comprises a molecule or a protein construct of the invention as defined herein and the use of such molecules or protein constructs as molecular tools, for example in in vitro or in vivo assays.

The protein constructs, e.g. recombinant protein constructs of the invention, have clear therapeutic uses. For example, the protein constructs, e.g. recombinant protein constructs, of the invention can be used to treat or prevent any disease which will benefit from treatment with therapeutic molecules which can bind to IgE, i.e. can be used in any anti-IgE therapy. As the molecules of the invention target and preferably eliminate IgE, preferred diseases for treatment with the constructs of the present invention are those diseases or conditions mediated by or associated with, or characterized by, high or abnormal levels of IgE, e.g. high or abnormal levels of free or soluble IgE which are present in the circulation or tissues at high or abnormal (e.g. unusually high or very high or pathological) concentrations, for example concentrations which are too high for effective treatment with conventional anti-IgE antibodies. As described elsewhere herein, the protein constructs of the invention can also be used to target and eliminate, or kill, cells expressing membrane IgE, e.g. membrane IgE expressing B cells, plasmablasts or plasma cells. In such embodiments, conveniently a component, e.g. an additional component, can be included in the construct, or attached to the construct which can confer cell killing activity into the molecule or construct, for example by enabling ADCC activity or other types of cell killing, e.g. using payloads or toxins. Thus, the protein constructs of the invention can be used to treat or prevent any disease which will benefit from the reduction, removal or killing of such cells expressing membrane IgE.

Thus the present invention further provides a protein construct, preferably a recombinant protein construct, of the invention for use in therapy.

Thus the present invention further provides a protein construct, preferably a recombinant protein construct, of the invention for use in the treatment or prevention of any IgE related disease or condition, e.g. for use in the treatment or prevention of any disease or condition which will benefit from reducing levels of IgE, or the treatment or prevention of any disease or condition associated with or characterised by elevated or abnormal (e.g. abnormally increased) levels of IgE or cells expressing membrane IgE. Examples of specific diseases or conditions which might be treated or prevented using the protein constructs of the invention include allergic disease and asthma (including allergic and non-allergic asthma).

The term “allergic disease” is to be understood according to its meaning in the art of medicine. In particular, allergic disease within the meaning of the invention includes a disease that is characterized by an allergic and/or atopic immunological reaction to an antigen, which results in allergic and/or atopic symptoms in the patient suffering from allergic disease. The term “allergic disease” in particular includes a disease which is characterized by elevated circulating IgE levels. An allergic disease often is characterized by the generation of antigen-specific IgE and the resultant effects of the IgE antibodies. As is well-known in the art, IgE binds to IgE receptors on mast cells and basophils. Upon later exposure to the antigen recognized by the IgE, the antigen cross-links the IgE on the mast cells and basophils causing degranulation of these cells.

Preferred examples of allergic disease are allergic asthma, allergic rhinitis, such as seasonal allergic rhinitis and perennial allergic rhinitis, and atopic dermatitis.

Allergic and non-allergic asthma is a clinical disorder that is characterized by airway inflammation; airway obstruction, which is reversible; and increased sensitivity, referred to as hyperreactivity. Obstruction to airflow is measured by a decrement in forced expired volume in one second (FEV I) which is obtained by comparison to baseline spirometry. Hyperreactivity of the airways is recognized by decreases in FEVI in response to very low levels of histamine or methacholine. Hyperreactivity may be exacerbated by exposure of the airways to allergen. Allergy testing can be helpful in identifying allergens in patients with persistent asthma. Common allergens include pet dander, dust mites, cockroach allergens, molds, and pollens. Common respiratory irritants include tobacco smoke, pollution, and fumes from burning wood or gas.

Allergic rhinitis is a clinical disorder characterized by nasal congestion, rhinorrhea, sneezing, and itching. Severity of these symptoms can vary from year to year, with occasional spontaneous remissions. Therefore, allergic rhinitis is classified by whether symptoms occur during certain seasons (SAR or seasonal allergic rhinitis) or year-round (PAR or perennial allergic rhinitis). The seasonal variety is usually caused by pollens from plants that depend on the wind for cross-pollination, such as grasses, trees, weeds, and mold spores. Serious complications, such as nasal polyps, recurrent sinusitis, recurrent ear infections, and hearing loss, can occur if allergic rhinitis is not treated or is undertreated. Psychosocial effects can include frequent absences from work or school, poor performance, poor appetite, malaise, and chronic fatigue.

Atopic dermatitis is a skin disorder involving hypersensitivity reaction within the skin characterized by inflammation, itching, and scaling. Atopic dermatitis can occur in an infantile or adult form. There is often a family history of asthma, hay fever, eczema, psoriasis, or other allergic diseases or allergy-related disorders. In adults, it is generally a chronic condition. Neurodermatitis is also a form of atopic dermatitis and can be treated. It is characterized by a self-perpetuating scratch-itch cycle. Although symptoms increase in times of stress, physiological changes in the nerve fibers are also present. A hypersensitivity reaction occurs in the skin, causing chronic inflammation.

Other diseases suitable for treatment include other hyper-IgE syndromes, allergic bronchopulmonary aspergilliosis and other aspergilliosis related conditions, Idiopathic Anaphylaxis, Anaphylaxis, Bullous pemphigoid, Pemphigus vulgaris, Urticaria, e.g. Chronic urticaria, Nasal polyposis, Chronic sinusitis, Mastocytosis and other mast cell disorders, Atopic keratoconjunctivitis, Eosinophil diseases of the GI tract including Eosinophilic gastroenteritis, Ulcerative colitis, Inflammatory bowel disease, Coeliac Disease and Crohns Disease.

Allergies to foodstuff (food allergies), including but not limited to; peanut, milk, wheat, soy, egg, peach, kiwi, sesame, seafood, fish etc can also be treated, in addition to IgE-related allergic sensitivity to non-food related substances including venoms from insects, wasps, bees or spiders, therapeutic drugs including antibiotic and chemotherapeutic agents, radioactive agents, latex, rubber and other potentially allergic materials.

Autoimmune and inflammatory indications where IgE may play a role can also be treated, including but not limited to lupus nephritis, SLE, multiple sclerosis, Chronic bronchitis, Chronic obstructive pulmonary disorder, Rheumatoid arthritis, Neuroinflammatory disorders. These diseases are thus also suitable for treatment with the constructs of the invention.

The present invention further provides the use of a protein construct, preferably a recombinant protein construct, of the invention in the manufacture of a medicament or composition for use in therapy or for use in the treatment or prevention of any of the above mentioned diseases or conditions.

The present invention further provides a method of treatment or prevention of any of the above mentioned diseases or conditions wherein said method comprises the step of administering to a patient in need thereof a therapeutically effective amount of a protein construct, preferably a recombinant protein construct, of the invention.

Nucleic acid molecules or expression vectors of the invention can equally be used in the therapeutic methods as described herein.

The in vivo methods and uses as described herein are generally carried out in a mammal. Any mammal may be treated, for example humans and any livestock, domestic or laboratory animal. Specific examples include mice, rats, pigs, cats, dogs, sheep, rabbits, horses, cows and monkey (e.g. cynomolgus monkey). Preferably, however, the mammal is a human. Another preferred mammal is canine (e.g. dog).

Thus, the term “patient” or “subject” as used herein includes any mammal, for example humans and any livestock, domestic or laboratory animal as described above. Preferably, however, the patient is a human subject. Thus, subjects or patients treated in accordance with the present invention will preferably be humans. Anther preferred subject or patient is canine (e.g. dog).

A therapeutically effective amount will be determined based on the clinical assessment and can be readily monitored.

The compositions and methods and uses of the present invention may be used in combination with other therapeutics and diagnostics.

The invention further includes kits comprising one or more of the protein constructs or compositions of the invention or one or more of the nucleic acid molecules encoding the protein constructs of the invention, or one or more expression vectors, e.g. recombinant expression vectors, comprising the nucleic acid molecules of the invention, or one or more host cells comprising the expression vectors, e.g. recombinant expression vectors, or nucleic acid molecules of the invention. Preferably said kits are for use in the methods and uses as described herein, e.g. in the therapeutic methods as described herein, or are for use in the in vitro assays or methods as described herein. Preferably said kits comprise instructions for use of the kit components. Preferably said kits are for treating or preventing diseases as described elsewhere herein, and optionally comprise instructions for use of the kit components to treat or prevent such diseases.

As used throughout the entire application, the terms “a” and “an” are used in the sense that they mean “at least one”, “at least a first”, “one or more” or “a plurality” of the referenced components or steps, except in instances wherein an upper limit is thereafter specifically stated.

In addition, where the terms “comprise”, “comprises”, “has” or “having”, or other equivalent terms are used herein, then in some more specific embodiments these terms include the term “consists of” or “consists essentially of”, or other equivalent terms.

Lists “consisting of” various components and features as discussed herein can also refer to lists “comprising” the various components and features.

The term “avidity” as used herein describes the combined strength of multiple bond interactions between proteins. Avidity is thus distinct from affinity which describes the strength of a single bond. As such, avidity is the combined synergistic (co-operative) strength of bond affinities rather than the sum of bonds and is sometimes referred to as functional affinity or relative affinity or overall affinity.

As used herein, the term “about” or “around” refers to variation in the numerical value that can occur, for example, through typical experimental error in measuring or determining these values depending on the method which is used. In some embodiments, the term “about” or “around” means within 10% of the reported numerical value, preferably within 5% or 2% of the reported numerical value.

The term protein or polypeptide as used herein refers to any molecule consisting of or comprising any type of amino acid. Thus molecules containing natural and/or non-natural or modified or synthetic amino acids are included. Similarly, the term nucleic acid molecule or nucleic acid as used herein refers to any molecule consisting of or comprising any type of nucleotide. Thus molecules containing natural and/or non-natural or modified or synthetic nucleotides are included.

The terms “decrease” or “reduce” (or equivalent terms) as referred to herein includes any measurable decrease or reduction when compared with an appropriate control. Preferably such decreases or reductions (and indeed other decreases, reductions or negative effects as mentioned elsewhere herein) are significant reductions, preferably clinically significant or statistically significant reductions, for example with a probability value of <0.05, when compared to an appropriate control level or value. Appropriate controls would readily be identified by a person skilled in the art and might include for example levels of a parameter or functional property observed in the absence of a construct of the invention in comparison to the presence of said construct (e.g. compared to an untreated sample), or in the absence (or presence) of a particular feature of a construct of the invention in comparison to the presence (or absence), as appropriate, of said feature.

The terms “increase” or “enhance” (or equivalent terms) as referred to herein include any measurable increase or enhancement or improvement when compared with an appropriate control. Preferably such increases (and indeed other improvements or positive effects as mentioned elsewhere herein) are significant increases, preferably clinically significant or statistically significant increases, for example with a probability value of <0.05, when compared to an appropriate control level or value. Appropriate controls would readily be identified by a person skilled in the art and might include for example levels of a parameter or functional property observed in the absence of a construct of the invention in comparison to the presence of said construct (e.g. compared to an untreated sample), or in the absence (or presence) of a particular feature of a construct of the invention in comparison to the presence (or absence), as appropriate, of said feature.

The terms “bind to”, “can bind to” and equivalent terms as used herein for various molecules or entities includes the ability to specifically bind to the relevant target.

Treatment of disease or conditions in accordance with the present invention (for example treatment of pre-existing disease) includes cure of said disease or conditions, or any reduction or alleviation of disease (e.g. reduction in disease severity) or symptoms of disease.

As will be clear from the disclosure elsewhere herein, the methods and uses of the prevent invention are suitable for prevention of diseases as well as active treatment of diseases (for example treatment of pre-existing disease). Thus, prophylactic treatment is also encompassed by the invention. For this reason in the methods and uses of the present invention, treatment also includes prophylaxis or prevention where appropriate.

Such preventative (or protective) aspects can conveniently be carried out on healthy or normal or at risk subjects and can include both complete prevention and significant prevention. Similarly, significant prevention can include the scenario where severity of disease or symptoms of disease is reduced (e.g. measurably or significantly reduced) compared to the severity or symptoms which would be expected if no treatment is given.

Some of the sequences referred to herein are summarised in the Table below, along with relevant identifiers.

| SEQ ID | | |
|---|---|---|
| NO: | Description | Sequence |
| 1 | CD23a | meegqyseie elprrrccrr gtqivllglv taalwagllt |
| | | llllwhwdtt qslkqleera |
| | | arnvsqvskn leshhgdqma qksqstqisq eleelraeqq |
| | | rlksqdlels wnlnglqadl |
| | | ssfksqelne rneasdller lreevtklrm elqvssgfvc |
| | | ntcpekwinf qrkcyyfgkg |
| | | tkqwvharya cddmegqlvs ihspeeqdfl tkhashtgsw |
| | | iglrnldlkg efiwvdgshv |
| | | dysnwapgep tsrsqgedcv mmrgsgrwnd afcdrklgaw |
| | | vcdrlatctp pasegsaesm |
| | | gpdsrpdpdg rlptpsaplh s |
| 2 | CD23b | mnppsqeiee lprrrccrrg tqivllglvt aalwaglltl |
| | | lllwhwdttq slkqleeraa |
| | | rnvsqvsknl eshhgdqmaq ksqstqisqe leelraeqqr |
| | | lksqdlelsw nlnglqadls |
| | | sfksqelner neasdllerl reevtklrme lqvssgfvcn |
| | | tcpekwinfq rkcyyfgkgt |
| | | kqwvharyac ddmegqlvsi hspeeqdflt khashtgswi |
| | | glrnldlkge fiwvdgshvd |
| | | ysnwapgept srsqgedcvm mrgsgrwnda fcdrklgawv |
| | | cdrlatctpp asegsaesmg |
| | | pdsrpdpdgr lptpsaplhs |
| 3 | D48 to S321 | dtt qslkqleera |
| | | arnvsqvskn leshhgdqma qksqstqisq eleelraeqq |
| | | rlksqdlels wnlnglqadl |
| | | ssfksqelne rneasdller lreevtklrm elqvssgfvc |
| | | ntcpekwinf qrkcyyfgkg |
| | | tkqwvharya cddmegqlvs ihspeeqdfl tkhashtgsw |
| | | iglrnldlkg efiwvdgshv |
| | | dysnwapgep tsrsqgedcv mmrgsgrwnd afcdrklgaw |
| | | vcdrlatctp pasegsaesm |
| | | gpdsrpdpdg rlptpsaplh s |
| 4 | Q81 to S321 | qksqstqisq eleelraeqq rlksqdlels wnlnglqadl |
| | | ssfksqelne rneasdller lreevtklrm elqvssgfvc |
| | | ntcpekwinf qrkcyyfgkg |
| | | tkqwvharya cddmegqlvs ihspeeqdfl tkhashtgsw |
| | | iglrnldlkg efiwvdgshv |
| | | dysnwapgep tsrsqgedcv mmrgsgrwnd afcdrklgaw |
| | | vcdrlatctp pasegsaesm |
| | | gpdsrpdpdg rlptpsaplh s |
| 5 | L102 to S321 | iksqdlels wnlnglqadl |
| | | ssfksqelne rneasdller lreevtklrm elqvssgfvc |
| | | ntcpekwinf qrkcyyfgkg |
| | | tkqwvharya cddmegqlvs ihspeeqdfl tkhashtgsw |
| | | iglrnldlkg efiwvdgshv |
| | | dysnwapgep tsrsqgedcv mmrgsgrwnd afcdrklgaw |
| | | vcdrlatctp pasegsaesm |
| | | gpdsrpdpdg rlptpsaplh s |
| 6 | V159 to P290 | vc ntcpekwinf qrkcyyfgkg |
| | | tkqwvharya cddmegqlvs ihspeeqdfl tkhashtgsw |
| | | iglrnldlkg efiwvdgshv |
| | | dysnwapgep tsrsqgedcv mmrgsgrwnd afcdrklgaw |
| | | vcdrlatctp |
| 7 | C160-C288 | c ntcpekwinf qrkcyyfgkg |
| | | tkqwvharya cddmegqlvs ihspeeqdfl tkhashtgsw |
| | | iglrnldlkg efiwvdgshv |
| | | dysnwapgep tsrsqgedcv mmrgsgrwnd afcdrklgaw |
| | | vcdrlatc |
| 8 | F170-L277 | f qrkcyyfgkg |
| | | tkqwvharya cddmegqlvs ihspeeqdfl tkhashtgsw |
| | | iglrnldlkg efiwvdgshv |
| | | dysnwapgep tsrsqgedcv mmrgsgrwnd afcdrkl |
| 9 | S156 to S321 | sgfvc ntcpekwinf qrkcyyfgkg |
| | | tkqwvharya cddmegqlvs ihspeeqdfl tkhashtgsw |
| | | iglrnldlkg efiwvdgshv |
| | | dysnwapgep tsrsqgedcv mmrgsgrwnd afcdrklgaw |
| | | vcdrlatctp pasegsaesm |
| | | gpdsrpdpdg rlptpsaplh s |
| 10 | E133 to A292 | easdller lreevtklrm elqvssgfvc ntcpekwinf |
| | | qrkcyyfgkg |
| | | tkqwvharya cddmegqlvs ihspeeqdfl tkhashtgsw |
| | | iglrnldlkg efiwvdgshv |
| | | dysnwapgep tsrsqgedcv mmrgsgrwnd afcdrklgaw |
| | | vcdrlatctp pa |
| 11 | E133 to E298 | easdller lreevtklrm elqvssgfvc ntcpekwinf |
| | | qrkcyyfgkg |
| | | tkqwvharya cddmegqlvs ihspeeqdfl tkhashtgsw |
| | | iglrnldlkg efiwvdgshv |
| | | dysnwapgep tsrsqgedcv mmrgsgrwnd afcdrklgaw |
| | | vcdrlatctp pasegsae |
| 12 | E133 to S321 | easdller lreevtklrm elqvssgfvc ntcpekwinf |
| | | qrkcyyfgkg |
| | | tkqwvharya cddmegqlvs ihspeeqdfl tkhashtgsw |
| | | iglrnldlkg efiwvdgshv |
| | | dysnwapgep tsrsqgedcv mmrgsgrwnd afcdrklgaw |
| | | vcdrlatctp pasegsaesm |
| | | gpdsrpdpdg rlptpsaplh s |
| 13 | S156 to E298 | sgfvc ntcpekwinf qrkcyyfgkg |
| | | tkqwvharya cddmegqlvs ihspeeqdfl tkhashtgsw |
| | | iglrnldlkg efiwvdgshv |
| | | dysnwapgep tsrsqgedcv mmrgsgrwnd afcdrklgaw |
| | | vcdrlatctp pasegsae |
| 14 | W184 to A279 | wvharya cddmegqlvs ihspeeqdfl tkhashtgsw |
| | | iglrnldlkg efiwvdgshv dysnwapgep tsrsqgedcv |
| | | mmrgsgrwnd afcdrklga |
| 15 | S156 to A292 | sgfvc ntcpekwinf qrkcyyfgkg |
| | | tkqwvharya cddmegqlvs ihspeeqdfl tkhashtgsw |
| | | iglrnldlkg efiwvdgshv |
| | | dysnwapgep tsrsqgedcv mmrgsgrwnd afcdrklgaw |
| | | vcdrlatctp pa |
| 16 | Linker (base | GGGGS |
| | unit) | |
| 17 | Linker (x3 | GGGGSGGGGSGGGGS |
| | repeats) | |
| 18 | Linker (x6 | GGGGSGGGGSGGGGSGGGGSGGGGSGGGGS |
| | repeats) | |
| 19 | whole secreted | EASDLLERLREEVTKLRMELQVSSGFVCNTCPEKWI |
| | sequence of | NFQRKCYYFGKGTKQWVHARYACDDMEGQLVSIH |
| | hCD23-G4S3- | SPEEQDFLTKHASHTGSWIGLRNLDLKGEFIWVDGS |
| | mIgG2a Fc (signal | HVDYSNWAPGEPTSRSQGEDCVMMRGSGRWNDA |
| | peptide removed) | FCDRKLGAWVCDRLATCTPPASEGSAESMGPDSRP |
| | | DPDGRLPTPSAPLHSGGGGSGGGGSGGGGSASISAM |
| | | VRSPRGPTIKPCPPCKCPAPNLEGGPSVFIFPPKIKDV |
| | | LMISLSPIVTCVVVDVSEDDPDVQISWFVNNVEVHT |
| | | AQTQTHREDYNSTLRVVSALPIQHQDWMSGKAFA |
| | | CAVNNKDLPAPIERTISKPKGSVRAPQVYVLPPPEEE |
| | | MTKKQVTLTCMVTDFMPEDIYVEWTNNGKTELNY |
| | | KNTEPVLDSDGSYFMYSKLRVEKKNWVERNSYSCS |
| | | VVHEGLHNHHTTKSFSRTPGK |
| 20 | mouse kappa leader | MSVPTQVLGLLLLWLTDARCDGA |
| | (secretory signal | |
| | peptide) | |
| 21 | Whole ORF | MSVPTQVLGLLLLWLTDARCDGAEASDLLERLREE |
| | sequence of | VTKLRMELQVSSGFVCNTCPEKWINFQRKCYYFGK |
| | hCD23-G4S3- | GTKQWVHARYACDDMEGQLVSIHSPEEQDFLTKH |
| | mIgG2a Fc | ASHTGSWIGLRNLDLKGEFIWVDGSHVDYSNWAPG |
| | (including | EPTSRSQGEDCVMMRGSGRWNDAFCDRKLGAWV |
| | secretory signal | CDRLATCTPPASEGSAESMGPDSRPDPDGRLPTPSA |
| | peptide) | PLHSGGGGSGGGGSGGGGSASISAMVRSPRGPTIKP |
| | | CPPCKCPAPNLEGGPSVFIFPPKIKDVLMISLSPIVTC |
| | | VVVDVSEDDPDVQISWFVNNVEVHTAQTQTHRED |
| | | YNSTLRVVSALPIQHQDWMSGKAFACAVNNKDLP |
| | | APIERTISKPKGSVRAPQVYVLPPPEEEMTKKQVTLT |
| | | CMVTDFMPEDIYVEWTNNGKTELNYKNTEPVLDS |
| | | DGSYFMYSKLRVEKKNWVERNSYSCSVVHEGLHN |
| | | HHTTKSFSRTPGK |
| 22 | part of neck/stalk | EASDLLERLREEVTKLRMELQVS |
| | region | |
| 23 | human der CD23 | SGFVCNTCPEKWINFQRKCYYFGKGTKQWVHARY |
| | start | ACDDMEGQLVSIHSPEEQDFLTKHASHTGSWIGLR |
| | | NLDLKGEFIWVDGSHVDYSNWAPGEPTSRSQGEDC |
| | | VMMRGSGRWNDAFCDRKLGAWVCDRLATCTPPA |
| 24 | CD21 binding | SEGSAE |
| | region | |
| 25 | CD23 tail | SMGPDSRPDPDGRLPTPSAPLHS |
| 26 | mouse IgG2a Fc from | ASISAMVRSPRGPTIKPCPPCKCPAPNLEGGPSVFIFP |
| | Invivogen vector | PKIKDVLMISLSPIVTCVVVDVSEDDPDVQISWFVN |
| | mIgG2aet-Fc | NVEVHTAQTQTHREDYNSTLRVVSALPIQHQDWM |
| | (containing mutations | SGKAFACAVNNKDLPAPIERTISKPKGSVRAPQVYV |
| | L235E + E318A/K320A/ | LPPPEEEMTKKQVTLTCMVTDFMPEDIYVEWTNNG |
| | K322A with reference | KTELNYKNTEPVLDSDGSYFMYSKLRVEKKNWVE |
| | to the whole IgG heavy | RNSYSCSVVHEGLHNHHTTKSFSRTPGK |
| | chain sequence in order | |
| | to knock out binding to | |
| | the Fc-gamma | |
| | receptors CD16, CD32 | |
| | and CD64) | |
| 27 | IgG1_human | ASTKGPSVFPLAPSSKSTSGGTAALGCLVKDYFPEPVTV |
| | Fc\|P01857\|1- | SWNSGALTSGVHTFPAVLQSS |
| | 330 | GLYSLSSWTVPSSSLGTQTYICNVNHKPSNTKVDKKVE |
| | | PKSCDKTHTCPPCPAPELLGG |
| | | PSVFLFPPKPKDTLMISRTPEVTCVVVDVSHEDPEVKFN |
| | | WYVDGVEVHNAKTKPREEQYN |
| | | STYRWSVLTVLHQDWLNGKEYKCKVSNKALPAPIEKTI |
| | | SKAKGQPREPQVYTLPPSRDE |
| | | LTKNQVSLTCLVKGFYPSDIAVEWESNGQPENNYKTTPP |
| | | VLDSDGSFFLYSKLTVDKSRW |
| | | QQGNVFSCSVMHEALHNHYTQKSLSLSPGK |
| 28 | IgG2_human | ASTKGPSVFPLAPCSRSTSESTAALGCLVKDYFPEPVTV |
| | Fc\|P01859\|1- | SWNSGALTSGVHTFPAVLQSS |
| | 326 | GLYSLSSVVTVPSSNFGTQTYTCNVDHKPSNTKVDKTVE |
| | | RKCCVECPPCPAPPVAGPSVF |
| | | LFPPKPKDTLMISRTPEVTCVWDVSHEDPEVQFNWYVD |
| | | GVEVHNAKTKPREEQFNSTFR |
| | | VVSVLTVVHQDWLNGKEYKCKVSNKGLPAPIEKTISKTK |
| | | GQPREPQVYTLPPSREEMTKN |
| | | QVSLTCLVKGFYPSDISVEWESNGQPENNYKTTPPMLDS |
| | | DGSFFLYSKLTVDKSRWQQGN |
| | | VFSCSVMHEALHNHYTQKSLSLSPGK |
| 29 | IgG3_human | ASTKGPSVFPLAPCSRSTSGGTAALGCLVKDYFPEPVTV |
| | Fc\|P01860\|1- | SWNSGALTSGVHTFPAVLQSS |
| | 377 | GLYSLSSVVTVPSSSLGTQTYTCNVNHKPSNTKVDKRVE |
| | | LKTPLGDTTHTCPRCPEPKSC |
| | | DTPPPCPRCPEPKSCDTPPPCPRCPEPKSCDTPPPCPRC |
| | | PAPELLGGPSVFLFPPKPKDT |
| | | LMISRTPEVTCVVVDVSHEDPEVQFKWYVDGVEVHNAKT |
| | | KPREEQYNSTFRVVSVLTVLH |
| | | QDWLNGKEYKCKVSNKALPAPIEKTISKTKGQPREPQVY |
| | | TLPPSREEMTKNQVSLTCLVK |
| | | GFYPSDIAVEWESSGQPENNYNTTPPMLDSDGSFFLYSK |
| | | LTVDKSRWQQGNIFSCSVMHE |
| | | ALHNRFTQKSLSLSPGK |

All sequences in this Table and elsewhere herein are recited in the direction of N-terminal residue to C-terminal residue, or 5′ to 3′, in line with convention in this technical field. One or more, or any, of the above sequences, or fragments or variants thereof, for example sequences with at least 70%, 75%, 80% etc., identity thereto as described elsewhere herein, can be used in the constructs of the present invention. For example, SEQ ID NOs: 19 to 26 are used in the exemplified constructs together with linkers of SEQ ID NO:17 and linkers with 4× G4S repeats (i.e. GGGGS ×4).

The invention will be further described with reference to the following non-limiting Examples with reference to the following drawings in which:

FIG. 1: Depiction of “Biologic” comprising two sCD23 monomers attached via a linker to FcRn binding Fc fragment from an IgG together with a depiction of a mode of binding to IgE.

FIG. 2: The schematic depicts the predicted mechanism of action of the biologic. It demonstrates uptake of the biologic in complex with IgE through endocytosis or micro-pinocytosis. Within the early endosome, there is a reduction in intra-endosomal calcium and pH. The change in calcium concentration from the high levels found in serum to the much lower levels found in the endosome results in release of IgE by the biologic. The change of pH within the endosome to become acidic increases the affinity of IgG-Fc for FcRn, such that the biologic binds FcRn. Binding to FcRn permits the biologic to enter the recycling pathway to be returned to the serum. Meanwhile, the IgE cargo enters the lysosomal degradation pathway to be degraded.

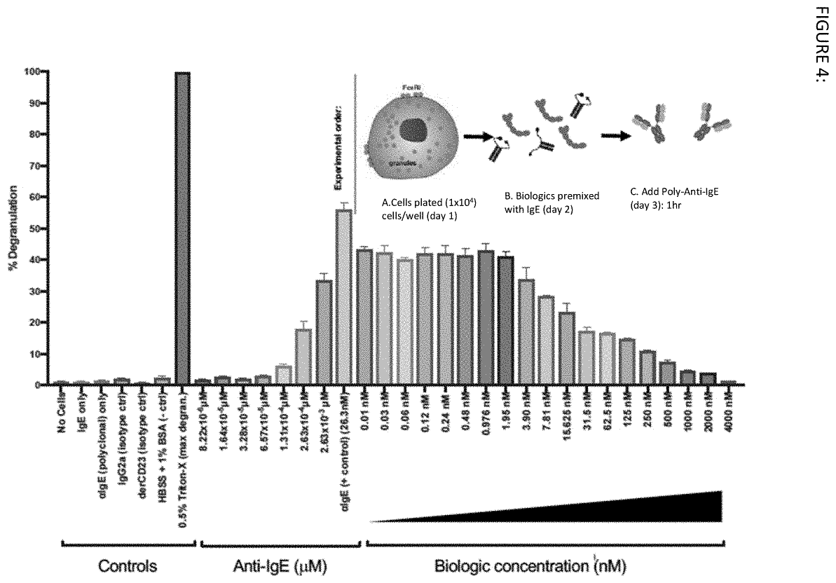

FIG. 3: An assay to assess the propensity of biologic (anti-IgE³) to potentiate degranulation of basophils pre-loaded and sensitized with IgE. Addition of a poly-clonal anti-IgE antibody to bind and cross-link surface FcεRI bound IgE resulted in degranulation as measured by β hexosaminidase release, which increased as the amount of cross-linking IgE was increased. In the presence of increasing concentration of biologic between 0.01 nM and 4 mM, there was no indication of basophil degranulation as measured by release of β hexosaminidase. A Triton X-100 control to completely lyse the basophils was used to indicate the maximum possible β hexosaminidase release (100%). This Figure shows that even at the highest concentration the biologic does not induce degranulation.

FIG. 4: An assay to assess the potential of the biologic (anti-IgE³) to inhibit IgE-mediated degranulation of basophilic RBL-SX38 cells. The cells were incubated in the presence of 1 nM IgE in the presence of a dose range of biologic overnight or up to 24 hours. The following day, a polyclonal anti-IgE was added in order to cross-link surface IgE-bound to FcεRI and potentiate the release of β hexosaminidase, which was subsequently measured as a means to quantify the level of cell degranulation. At biologic concentrations greater than, or equivalent to 1 nM IgE, there is a dose-dependent reduction in the amount of β hexosaminidase released by the RBL-SX38 cells. Addition of a poly-clonal anti-IgE antibody to bind and cross-link surface FcεRI bound IgE resulted in degranulation as measured by β hexosaminidase release, which increased as the amount of cross-linking IgE was increased. A Triton X-100 control to completely lyse the cells was used to indicate the maximum possible β hexosaminidase release (100%). This Figure shows that as biologic concentration is increased, IgE is prevented from binding to FcεRI, and sensitisation of basophils is inhibited.

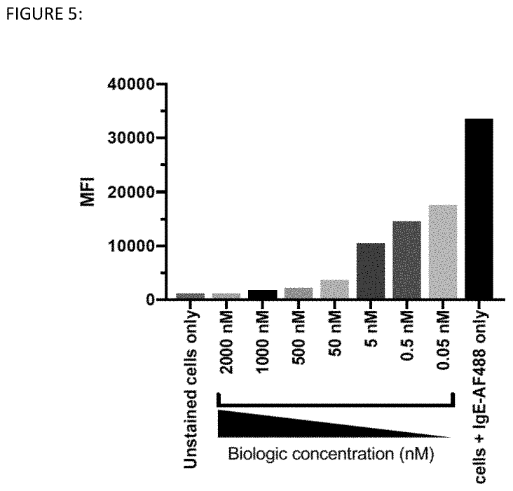

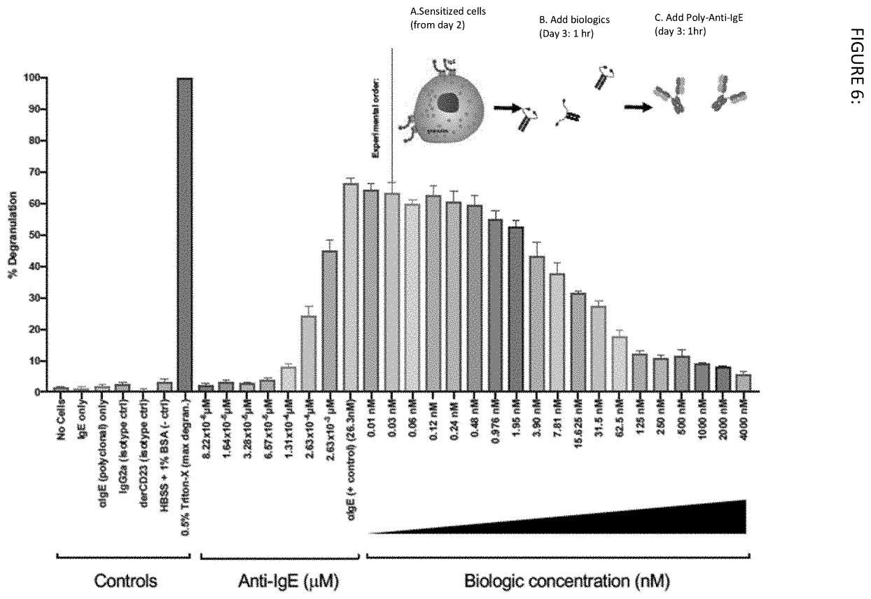

FIG. 5: The data shows the ability of biologic (anti-IgE³) to block IgE binding to RBL-SX38 cells expressing FcεRI. The cells were incubated with 1 nM IgE labelled with AF-488 either in the presence or absence of increasing concentrations of biologic between 0.05 to 2000 nM for 1 hour. The quantity of AF-488-labelled IgE present on the surface of the RBL-SX38 basophilic cells was quantified by FACS and presented as Mean Fluorescence Index. This Figure shows that as biologic concentration is increased, IgE is prevented from binding to FcεRI, and sensitisation of basophils is inhibited.

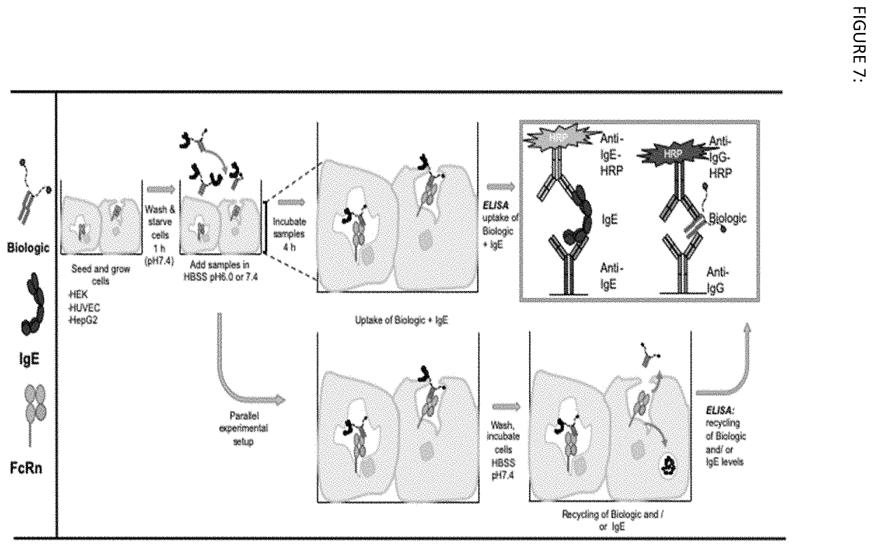

FIG. 6: The data shows the ability of the biologic (anti-IgE³) to block polyclonal anti-IgE induced RBL-SX38 basophilic cell degranulation when the cells have been pre-sensitized with FcεRI-bound IgE. RBL-SX38 cells were plated in appropriate medium and grown prior to the addition of IgE on day 2, then left 24 hours. On day 3, increasing quantities of biologic were then added to the cells and incubated with the cells for 1 hour, prior to the addition of a fixed quantity of cross-linking polyclonal anti-IgE to induce degranulation, as measured by the release of β hexosaminidase. Addition of a polyclonal anti-IgE antibody to bind and cross-link surface Fc□RI bound IgE resulted in degranulation as measured by β hexosaminidase release, which increased as the amount of cross-linking IgE was increased. A Triton X-100 control to completely lyse the cells was used to indicate the maximum possible β hexosaminidase release. This Figure shows that at high concentrations of biologic, degranulation was inhibited in presensitised basophils.

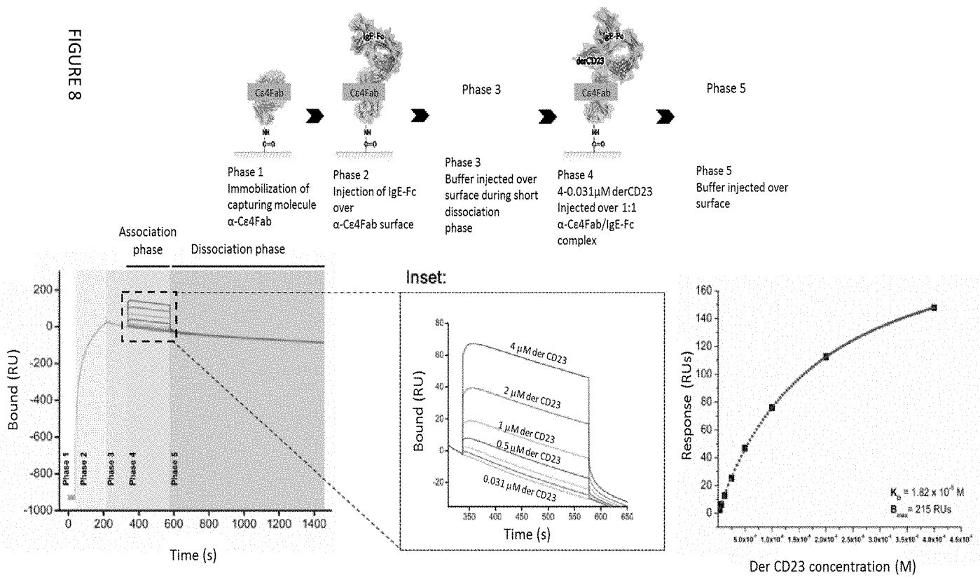

FIG. 7: The schematic describes the layout of the recycling and degradation assay, modified from Grevy's et al 2018. Briefly, HEK293 cells transfected with FcRn and β2 microglobulin were seeded and grown until a confluent intact monolayer was established. Cells were starved briefly prior to the addition of the test antibodies and proteins (IgE, biologic or biologic+protein) and then incubated in warm HBSS for 4 hours. The study was set up in parallel. To one half, following the incubation period, the supernatant was removed and the amount of IgE or biologic remaining was assessed by ELISA. The cells in these wells were then lysed and the intra-cellular uptake of IgE and biologic assessed by ELISA. In the other half of the study, the cells were washed extensively before a further 4-hour incubation period, to allow for ligand release back into the supernatant. Samples of supernatant were measured by ELISA for biologic or IgE, as well as the cells lysed to assess the amount internalised within the cell. The assay allows for the assessment of uptake of antibody-ligand complexes and the propensity of those complexes to be recycled, or to enter the lysosomal degradation pathway.

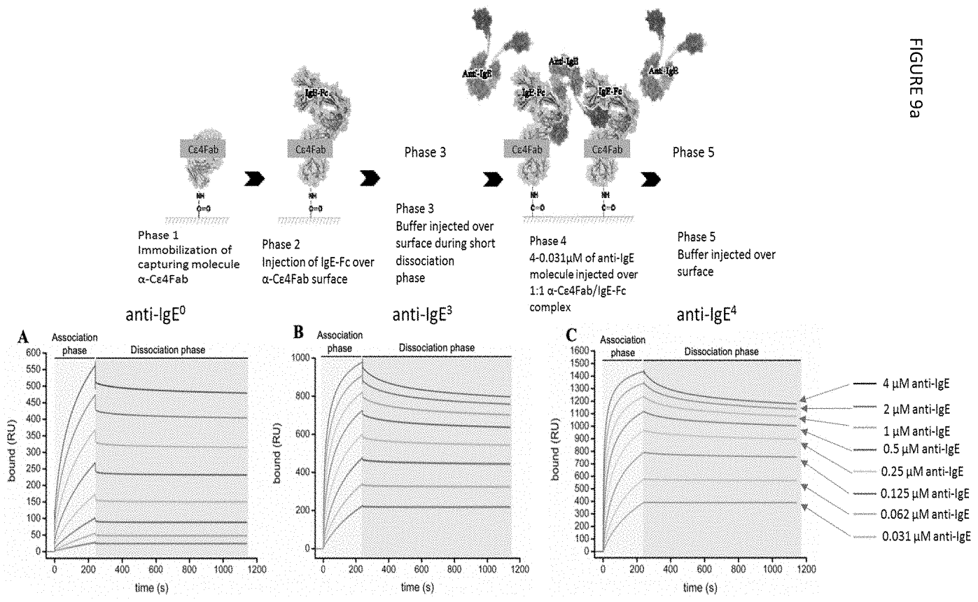

FIG. 8: The top panel shows a schematic representation of the experimental set-up for the surface plasmon resonance experiment. Below the schematic illustration is a sensorgram demonstrating binding of derCD23 monomer to IgE-Fc, which includes highlighting of the five different phases of the experiment. The inset corresponds to Phase 4 of the SPR binding profile shown in the top panel and demonstrates the binding profile of derCD23 across a range of different starting concentrations. Association and dissociation of derCD23 was rapid, and the interaction reached steady state within seconds of derCD23 injection. Double reference blank-subtracted data for derCD23 binding to α-Cε4 Fab captured IgE-Fc. Steady state binding curve analysis performed on the interaction between derCD23 and the 1:1 α-Cε4 Fab/IgE-Fc complex. The data fitted well to a one-to-one binding model over a concentration range of 0-4 μM, suggesting that only one derCD23-binding site was occupied on IgE, with an estimated K_{D} of 1.82×10⁻⁶ M.

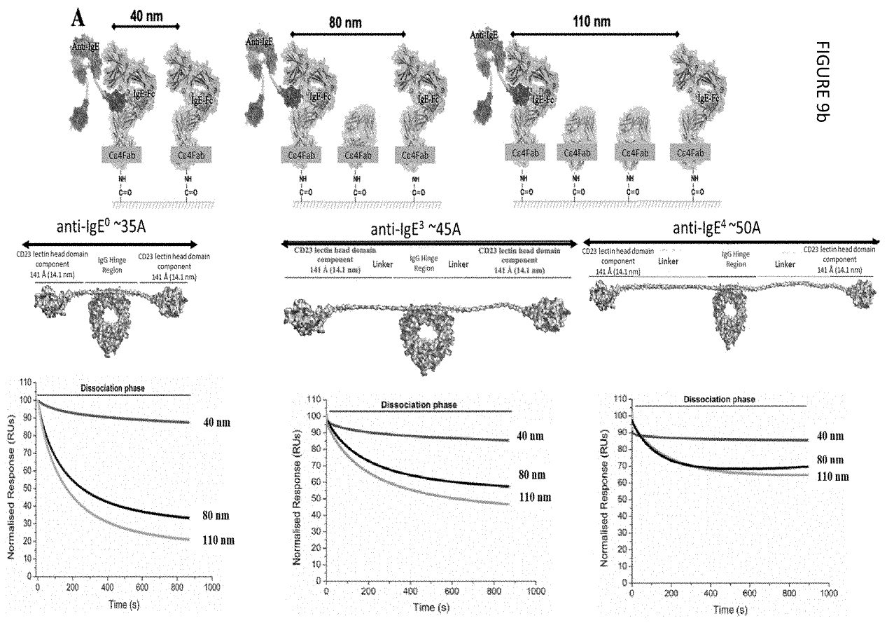

FIG. 9a: The top panel shows a schematic representation of the experimental set-up for the surface plasmon resonance experiment. SPR sensor surfaces were prepared by covalently conjugating α-Cε4 Fab via amine coupling (phase 1). Approximately 80 nM of IgE-Fc was injected over the α-Cε4 Fab surface, forming a 1:1 α-Cε4 Fab/IgE-Fc complex (phase 2). Following a short buffer injection, inducing a short dissociation phase, a range of concentrations in a two-fold dilution series of: anti-IgE⁰; anti-IgE³; and anti-IgE⁴; was flowed over the IgE-Fc, with 4 μM as the highest concentration. Lastly, approximately 800 s of buffer was flowed over the surface, inducing a dissociation phase. SPR sensorgrams depicting the binding and dissociation (phases 4 & 5) of the anti-IgE molecules to the 1:1 α-Cε4 Fab captured IgE-Fc complex. Blank subtracted sensorgrams for (A) anti-IgE⁰, (B) anti-IgE³ and (C) antidgE⁴ molecules binding to the 1:1 α-Cε4 Fab/IgE-Fc complex.

FIG. 9b: Dissociation phase comparison for the anti-IgE molecules when IgE-Fc is immobilised with increasing inter-molecular spacing is demonstrated by surface plasmon resonance. Molecular models for each of the biologic constructs; IgE⁰, IgE³ and IgE⁴ were constructed using the model building program Coot (Emsley et al., 2010). Images depicting the approximate structure (and inter CTLD separation) for each anti-IgE biologic were generated with PyMOL. IgE-Fc was immobilised at a concentration of 40 μM, which according to plating density calculations create an average molecular spacing of 110 nm. Similarly, an immobilised concentration of 80 nM and 160 μM were calculated to result in an average molecular spacing of 40 nm and 80 nm respectively. Following a short buffer injection, inducing a short dissociation phase, a range of concentrations in a two-fold dilution series of: anti-IgE⁰; anti-IgE³; and anti-IgE⁴; was flowed over the IgE-Fc, with 4 μM as the highest concentration. Lastly, approximately 800 s of buffer was flowed over the surface, inducing a dissociation phase. A comparison of the dissociation phase for each construct is depicted suggesting that linker length is a determinant of IgE-Fc binding properties.

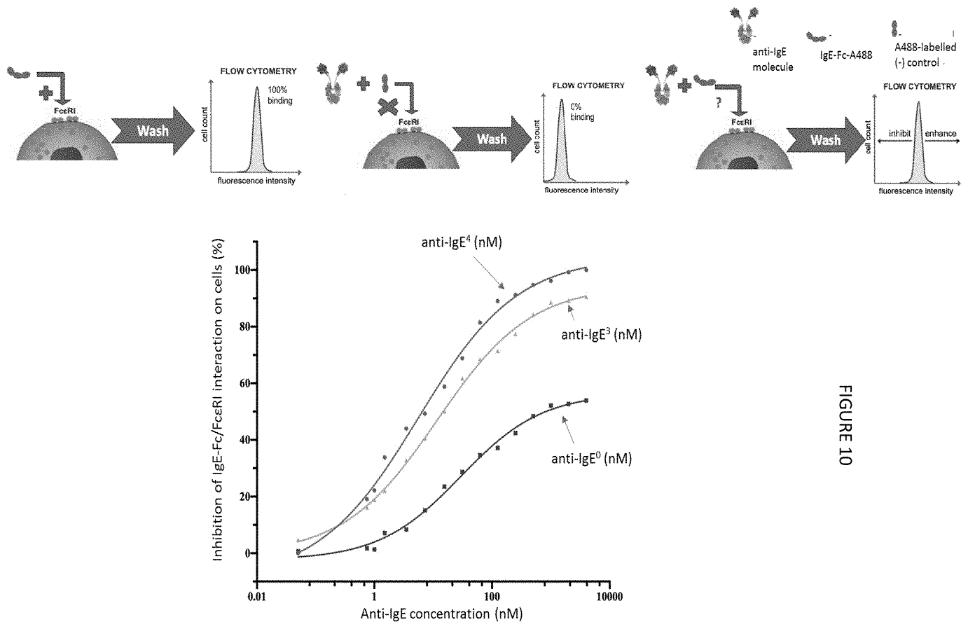

FIG. 10: The outline for the experiment is depicted schematically at the top of the Figure. IgE-Fc was fluorescently labelled with Alexa-488 (A488) and incubated with RBL SX-38 cells. The A488 fluorescence of single live cells was measured using flow cytometry. Observed A488 fluorescence intensity for binding of 1 nM IgE-Fc-A488 only was defined as 100% binding. A488 fluorescence intensities of single RBL SX-38 cells incubated with an A488-labelled negative control and 4000 nM of the three anti-IgE molecules were used to define 0% binding. The anti-IgE molecules, IgE⁰, IgE³ and IgE⁴ were incubated with IgE-Fc-A488 and RBL SX-38 cells at different concentrations (0-4000 nM) and their effect on the A488 fluorescence intensity, of single live cells binding to IgE-Fc-A488 was measured using flow cytometry. The study demonstrates the importance of linker length in determining the functional properties of the anti-IgE biologic.

### EXAMPLES

### Example 1: Cloning, Expression and Purification of Biologic Anti-IgE Construct

Method for cloning mouse kappa leader-CD23-(GGGGS)₃-Fc into pcDNA5-FRT The following sequence was synthesised as a double stranded gBlock DNA fragment by Integrated DNA Technologies (IDT):

(SEQ ID NO: 34)
atgagtgtgcccactcaggtcctggggttgctgctgctgtggcttacag
atgccagatgtgatggcgccgaagcttccgacctgctggaacggctgcg
ggaggaagtgaccaagctgcggatggaactgcaggtgtccagcggcttc
gtgtgcaacacctgccccgagaagtggatcaacttccagcggaagtgct
actacttcggcaagggcaccaagcagtgggtgcacgccagatacgcctg
cgacgacatggaaggccagctggtgtccatccacagccccgaggaacag
gacttcctgaccaagcacgccagccacaccggcagctggatcggcctgc
ggaacctggacctgaagggcgagttcatctgggtggacggcagccacgt
ggactacagcaactgggcccctggcgagcccacctccagaagccagggc
gaggactgcgtgatgatgcggggcagcggccggtggaacgacgccttct
gcgaccggaagctgggcgcctgggtgtgcgaccggctggccacctgcac
cccccctgccagcgagggcagcgccgagagcatgggccccgacagcagg
cccgaccccgacggcagactgcccacccccagcgcccctctgcacagcg
gcggcggcggcagcggcggcggcggcagcggcggcggcggcagcgccag
catatcggccatggttagatctcccagagggcccacaatcaagccctgt
cctccatgcaaatgcccagcacctaacctcgagggtggaccatccgtct
tcatcttccctccaaagatcaaggatgtactcatgatctccctgagccc
catagtcacatgtgtggtggtggatgtgagcgaggatgacccagatgtc
cagatcagctggtttgtgaacaacgtggaagtacacacagctcagacac
aaacccatagagaggattacaacagtactctccgggtggtcagtgccct
ccccatccagcaccaggactggatgagtggcaaggcgttcgcatgcgcg
gtcaacaacaaagacctcccagcgcccatcgagagaaccatctcaaaac
ccaaagggtcagtaagagctccacaggtatatgtcttgcctccaccaga
agaagagatgactaagaaacaggtcactctgacctgcatggtcacagac
ttcatgcctgaagacatttacgtggagtggaccaacaacgggaaaacag
agctaaactacaagaacactgaaccagtcctggactctgatggttctta
cttcatgtacagcaagctgagagtggaaaagaagaactgggtggaaaga
aatagctactcctgttcagtggtccacgagggtctgcacaatcaccaca
cgactaagagcttctcccggactccgggtaaatga

This was then cloned by PIPE cloning into pcDNA5-FRT (ThermoFisher). Briefly, the vector was linearised by PCR (Pfu, Promega) using the primers gtctgtgtgtgatcagtgtgaggctg (SEQ ID NO:35) and taagataaacctgcctccctccctcccagggctccatccagctgtg (SEQ ID NO:36), purified by gel extraction and treated with DpnI (ThermoFisher) to remove the original plasmid. The insert was amplified by PCR (Phusion Flash, ThermoFisher) from the gBlock using the primers tgatcacacacagacatgagtgtgcccactca (SEQ ID NO:37) and gagggaggcaggtttatcttatcatttacccggagtccgggaga (SEQ ID NO:38) which have overhangs homologous to the ends of the vector, then purified by gel extraction. Products were mixed in 1:1, 2:1 or 1:2 ratios, incubated at room temperature for 30 mins and then used to transform NEB10β competent E. coli (NEB). Colonies were grown up in LB-amp and the plasmid DNA miniprepped (Monarch kit, NEB), then sequenced in full (Eurofins).

The translated protein sequence is shown below and includes a secretory signal peptide labelled as ‘mouse kappa leader’ that is cleaved during processing in the mammalian HEK293 cells used for protein expression.

(SEQ ID NO: 21)
MSVPTQVLGLLLLWLTDARCDGAEASDLLERLREEVTKLRMELQVSSGF
VCNTCPEKWINFQRKCYYFGKGTKQWVHARYACDDMEGQLVSIHSPEEQ
DFLTKHASHTGSWIGLRNLDLKGEFIWVDGSHVDYSNWAPGEPTSRSQG
EDCVMMRGSGRWNDAFCDRKLGAWVCDRLATCTPPASEGSAESMGPDSR
PDPDGRLPTPSAPLHS [symbol] ASISAMVRSPRGPTIKP
CPPCKCPAPNLEGGPSVFIFPPKIKDVLMISLSPIVTCVVVDVSEDDPD
VQISWFVNNVEVHTAQTQTHREDYNSTLRVVSALPIQHQDWMSGKAFAC
AVNNKDLPAPIERTISKPKGSVRAPQVYVLPPPEEEMTKKQVTLTCMVT
DFMPEDIYVEWTNNGKTELNYKNTEPVLDSDGSYFMYSKLRVEKKNWVE
RNSYSCSVVHEGLHNHHTTKSFSRTPGK

Mouse Kappa Leader (bold), sCD23, (GGGGS)₃ linker (bold italic), mIgG-2AFc (underlined italic)

Co-Transfection of pcDNA5/FRT/CD23-IgGFc Vectors and pOG44 Flp-Recombinase Vector

As recommended for FuGene (Promega), briefly: the day before the transfection, half a 24-well plate (Flat round well, tissue culture 24-well Nunc™ plate, ThermoFisher) was plated at a density of 8×10⁴ cells/well with FlpIn HEK293 cells, and the second half at a density of 4×10⁴ cells/well in 500 μl of complete growth medium (DMEM+10% Fetal Bovine Serum). For a single protein transfection in duplicate, 0.11 ug of pcDNA5-FRT vector and 0.99 ug of pOG44 DNA (ThermoFisher) were added in a combined total volume of 52 μl of sterile deionized water. Using a 3:1 Fugene to DNA ratio, 3.3 μl of FuGene was carefully added. This was achieved by avoiding touching the sides of the microcentrifuge tube with the tip of the pipette. The solution was vortexed for 20 seconds, and spun-down to recover all the solution in the base of the microcentrifuge tube. After 10 minutes incubation at room temperature, 25 μl of complex was added per well of FlpIn HEK293 cells (one at the higher density and one at the lower cell density) and mixed thoroughly. Cells were returned to the 37° C. 5% CO₂ humidified incubator and after 48 hours the cells were split into 6-well plates containing complete media with 50 μg/ml hygromycin (ThermoFisher hygromycin B in PBS) for selection. A month later successfully transfected cells form loci in the wells which can be expanded, typically into a 1 L spinner flask or 5 L WAVE bioreactor and the culture supernatants harvested after two weeks.

Protein-G Affinity Purification

Cell supernatants were harvested and centrifuged at 4000×g for 15 minutes to remove cell debris. Supernatants were passed through 0.45 μm filters (Sartorius) and stored at 4° C. with 0.1% sodium azide (Sigma) until purification. The CD23-IgGFc fusion proteins were purified by affinity chromatography with a 5 ml HiTrap Protein-G HP column (GE Healthcare) using an ÄKTA Prime system (GE Healthcare). The column was equilibrated with 5 Column Volumes (CV) of washing buffer (PBS, pH 7.4). Filtered supernatant was loaded onto the column at a flow rate of 2 ml/min and the column washed with 10 CV washing buffer. The CD23-IgGFc fusion proteins were eluted with 0.1 M Glycine-HCl, pH 2.5 and 2.5 ml fractions were collected into tubes containing 0.5 ml 1M Tris-HCl pH 8.6 for neutralization.

Size-Exclusion Chromatography of Affinity Purified CD23-IgGFc Fusion Proteins

Size-exclusion chromatography was performed on a Gilson HPLC system using a Superdex™ 200 10/300 GL column (GE Healthcare), at a flow rate of 0.75 ml/min in PBS pH 7.4. The size-exclusion chromatography analysis showed no aggregation and confirmed the affinity column-purified product consists of monodisperse molecules of the expected size (˜100 KDa).

### Example 2: Assessment of the Effect of Biologic Anti-IgE on Basophil Degranulation

Degranulation assays were used to assess the propensity of IgE-sensitive effector cells such as basophils and mast cells to release intra-cellular mediators held within granules inside the cytoplasm. When allergen specific IgE on the surface of effector cells encounters its specific allergen in the environment, it permits cross-linking between the high affinity IgE receptor, FcεRI to activate downstream signalling events. This results in the release of intra-cellular granules containing inflammatory mediators into the local milieu resulting in a typical allergic reaction. The potential for an anti-IgE biologic to inhibit, or potentiate this response is evaluated in a series of modified basophil degranulation assays.

Materials & Methods

Basophil Degranulation Assay

Rat basophilic leukaemia cell line RBL-SX38 cells stably expressing the human tetrameric (αβγ₂) high-affinity IgE receptor, FcεRI [Dibbern, D A et al., J Immunol Methods 2003; 274: 37-45], (a kind gift from Prof. J-P. Kinet, Harvard University, Boston, Mass.) were stimulated by a variety of IgE-mediated triggers to assess degranulation, as measured by the release of β-hexosaminidase. The methodology used is essentially that described in Rudman et al Clin Exp Allergy 2011, 41(10): 1400-1413, and Weigand et al 1996, J. Immunol, 157:221-230, is briefly described here.

As controls, unstimulated cells were used. To quantify total β-hexosaminidase cellular content, cells were incubated with 0.5% Triton X-100+1% Bovine serum albumin (BSA) in a suitable buffer to complete lysis prior to quantification of β-hexosaminidase (100% release). As a negative control, unstimulated cells were incubated with 1% BSA in HBSS (+/−control IgG used at a concentration comparable to the test article) (0% baseline). A no cell control was also included.

RBL-SX38 basophilic cells were seeded at a density of 1×10⁴ cells/well in a 96-well plate in culture medium (DMEM, 10% FCS, 1.2 mg/mL Geneticin G418 (Invitrogen)) overnight, prior to sensitisation with the addition of 200 ng/mL IgE (NIP IgE, AbD Serotec, Kidlington, Oxford), isotype controls, or medium only and a further overnight incubation. Cells were washed 3× in stimulation buffer (HBSS+1% BSA) prior to stimulation for 1 hour at 37° C., either with control antibody, or rabbit polyclonal anti-IgE used to cross-link surface bound IgE (Dako). β-hexosaminidase was quantified from 50 μL culture supernatant, then diluted 1:1 in stimulation buffer before being transferred to a black 96-well plate. Each well on the plate already contained 50 μL of a fluorogenic substrate (1 mM 4-methylumbelliferyl N-acteyl-b-D-glucosaminide in 0.1% DMSO, 0.1% Triton X100, 200 mM citrate buffer pH4.5). Samples were incubated for 2 hours in the dark before being quenched with 100 μL 0.5M Tris. Plates were read with a Fluostar Omega microplate reader (350 nm excitation, 450 nm emission)(BMG Labtech, Offenburg, Germany). Degranulation was expressed as a percentage of Triton X-100 release and compared with unstimulated cells.

To Assess the Propensity for Anti-IgE Biologic to Induce Basophil Degranulation Alone The biologic construct was tested for its ability to potentiate IgE-mediated degranulation events through cross-linking of IgE already bound to the FcεRI IgE receptor.

Materials & Methods

RBL-SX38 basophilic cells were prepared and loaded with IgE over a 48-hour period as described in the above materials and methods (basophil degranulation assay) section. To the cells loaded with IgE, the biologic was added over a serial dilution range between 4 μM and 0.016 nM, incubated for 1 hour. Samples of the supernatant were then taken and processed as described to assess the concentration of β-hexosaminidase released as a signal of cell degranulation.

Results & Discussion

Following 1-hour incubation with a control cross-linking anti-IgE, RBL-SX38 basophilic cells were stimulated to release β-hexosaminidase in a dose-dependent response. By contrast, in the presence of increasing concentrations of biologic (using the same experimental conditions) there was no indication of any β-hexosaminidase release, indicating that the biologic was unable to potentiate basophil activation or degranulation in isolation (FIG. 3).

To Assess the Propensity for Anti-IgE Biologic to Block IgE from Binding FcεRI in a Competition Study and Prevent Degranulation of a Basophilic Cell Line

The studies explore the potential of the anti-IgE biologic to bind IgE and prevent it binding to the high affinity IgE receptor, FcεRI, so preventing IgE-dependent degranulation of a basophil cell-line, RBL-SX38.

Materials & Methods

RBL-SX38 basophilic cells were seeded as described above and left to incubate overnight. The following day, pre-mixed solutions comprising a set standard concentration of 200 ng/mL (1 nM) IgE were prepared with increasing concentrations of the biologic anti-IgE construct. The pre-mixed solutions were then immediately added to cells and left to incubate overnight, before being subjected to the stimulation protocol with polyclonal anti-IgE, as described in the degranulation assay section above.

Results & Discussion

The data shown in FIG. 4 demonstrate that the anti-IgE biologic was able to inhibit IgE mediated degranulation in a dose dependent manner, and is consistent with it having blocked binding of IgE to the high affinity IgE receptor, FcεRI.

To Demonstrate that Biologic Anti-IgE Prevents Binding of IgE to the High Affinity Receptor, FcεRI

These studies explore the potential of the anti-IgE biologic to bind IgE and prevent it binding to the high affinity IgE receptor, FcεRI.

Materials & Methods

RBL-SX38 basophilic cells were seeded as described above and left to incubate overnight. The following day, pre-mixed solutions comprising a set standard concentration of 200 ng/mL AlexaFluor-488-labelled IgE (1 nM) were prepared with increasing concentrations of the biologic anti-IgE construct. The pre-mixed solutions were then immediately added to cells and left to incubate for 1 hour before being washed twice and re-suspended in 1 mL of FACS buffer for analysis. Cells were analysed on an Attune N×T Acoustic Focusing Cytometer (Lasers: BRVX) (ThermoFisher) and the data was analysed in FlowJo version 10.2.

Results & Discussion

The data shown in FIG. 5 demonstrate that the anti-IgE biologic was able to bind IgE and dose-dependently prevent binding of IgE to the high affinity IgE receptor, FcεRI on the RBL-SX38 cells in a competition binding study. As the concentration of biologic anti-IgE was increased, fewer IgE molecules were able to bind surface FcεRI so demonstrating the ability of these molecules to inhibit IgE binding to its high affinity receptor.

To Demonstrate that Biologic Anti-IgE Prevents Degranulation of Basophils Already Pre-Sensitised with IgE Bound to the High Affinity Receptor FcεRI

IgE binds to FcεRI on the surface of mast cells and basophils. In the presence of multi-valent allergen, FcεRI-bound IgE cross-links the receptors to potentiate cell activation and the release of inflammatory cell mediators through a degranulation response. The biologic anti-IgE was tested for its ability to prevent the degranulation response of already pre-sensitized basophilic cells.

Materials & Methods

RBL-SX38 basophilic cells were seeded as described above and left to incubate overnight prior to addition of 200 ng/mL IgE (1 nM) as per the protocol described above and incubated for a further 24 hours. Increasing concentrations of the biologic anti-IgE construct were then added to the wells containing the cells and left to incubate for 1 hour, before being subjected to the stimulation protocol with 5000 ng/mL polyclonal anti-IgE, as described in the degranulation assay section above.

Results & Discussion

The data shown in FIG. 6 demonstrate that the anti-IgE biologic was able to dose-dependently prevent IgE mediated degranulation, as measured by β-hexosaminidase release when in modest excess (>1 nM), rapidly within 1 hour. Further increase of incubation time did not further change the level of inhibition of degranulation observed with the constructs (data not shown).

### Example 3: Recycling & Uptake Cellular Assays

Materials and Methods:

Preparation of HEK-mFcRn/β2m & HEK-hFcRn/β2m Cells

HEK293F (ThermoFisher) cells are a human embryonic kidney cell line. Cells were maintained in DMEM+10% Fetal Bovine Serum and transiently transfected with either mouse or human FcRn and β2m, using the Fugene (ThermoFisher) transfection reagent as per example 1 and were ready for use after ˜48 hours. Other cell lines such as HUVEC, HepG2, CACO2 and HMEC1 can also be successfully transfected in this way (not shown).

The FcRn and β2m expression vectors for mouse (mFcRnFix-pEGFP-N1 & mB2-M-PCB7) and human (hFcRnWT-pEGFP-N1 & hB2-M-PCB7) were a gift from Prof E.S. Ward and the FcRn vectors contain a cytoplasmic GFP which is additionally useful for FACS and fluorescence microscopy (not described).

### REFERENCES

mFcRnFix-pEGFP-N1 & mB2-M-PCB7
Engineering the Fc region of immunoglobulin G to modulate in vivo antibody levels Carlos Vaccaro, Jinchun Zhou, Raimund J Ober & E Sally Ward, Nat. Biotechnol., 23 (10):1283-1288. 2005.
hFcRnWT-pEGFP-N1 & hB2-M-PCB7
Visualizing the site and dynamics of IgG salvage by the MHC class I related receptor FcRn
R. J. Ober, C. Martinez, C. Vacarro, E. S. Ward, J. Immunol., vol. 172, pp. 2021-2029, 2004.

Cell Recycling Assay Protocol

The assay protocol is depicted in FIG. 7.

a. HEK293-FcRn/β2m cells are seeded and grown until confluent (95-100% confluency) −7.5×10⁵ seeded into 24-well plates per well (Costar) and cultured for 2 days in growth medium.

b. Media removed, and the cells were washed twice and starved for 1 h in Hank's balanced salt solution (HBSS) buffer (pH 7.4).

c. The protein of interest is diluted in HBSS (pH 7.4 or 6.0) and added to cells and incubated for 4 h to allow for uptake of the antibodies.

d. Medium is removed⁺ and cells are washed four times with ice cold HBSS (pH 7.4), thereafter fresh warm HBSS (pH 7.4) or growth medium without FBS and supplemented with MEM non-essential amino acids (ThermoFisher) was added.

e. Samples were incubated with fresh warm HBSS (pH 7.4) and collected at 4 h or overnight (for 4 h incubation), —this to allow for ligand release⁺⁺.

f. Cells are extensively washed with ice cold HBSS (pH 7.4) and lysed⁺⁺⁺.

g. The collected samples are analysed in ELISAs specific for IgG or IgE.

⁺Media taken off and read for remaining construct in solution

⁺⁺Media aspirated and read by ELISA determining amount of construct (ligand) released back into solution/media

⁺⁺⁺Cellular lysate analyzed for levels of construct internalized within cell via ELISA.

Preparation of Total Protein Lysates

Total protein lysates were obtained using the CelLytic M cell lysis Reagent (Sigma-Aldrich) or RIPA lysis buffer (ThermoFisher) supplied with a protease inhibitor cocktail (Sigma-Aldrich) or complete protease inhibitor tablets (Roche). The mixture was incubated with the cells on ice and a shaker for 10 min followed by centrifugation for 15 min at 10,000×g to remove cellular debris. Quantification of the amounts of IgG or IgE present in the lysates was done by ELISA as described below.

The derived values for recycling and residual amount for the biologic and IgE was used to calculate the amount being recycled and the amount retained within the cell.

Total IgG-Fc (Anti-Mouse) ELISA

IgG-Fc concentrations in cell culture supernatants were determined by ELISA using the following method.
a. First, the capture antibody, a goat anti-mouse IgG (Sigma), was diluted in carbonate-bicarbonate buffer to a final concentration of 1 μg/mL.
b. Next 100 μL of this coating solution was added to each well on a Maxisorp™ 96 well plate and incubated overnight at 4° C.
c. After overnight incubation, the coating solution was removed from the wells and 200 μL of blocking buffer, 2% Skim Milk/PBS+0.5% Tween®20 (PBS-T), was added to each well. Plates were incubated for 2 hours and then wells were washed twice with 250 μL of PBS-T.
d. Next, the IgG standard was diluted to 400 ng/mL in 50% culture media (same as cell culture media) and 50% PBS-T/1% Skim Milk (assay buffer) and serially diluted 1:2 in the well plate down to 0.78 ng/mL in duplicate so that each well had a final volume of 50 μL.
e. The remaining wells were given 25 μL of assay buffer and 25 μL of supernatants or diluted supernatants derived from cell cultures.
f. Standards and samples were incubated for 2 hours before wells were washed four times with 250 μL of PBS-T.
g. Next the secondary antibody, goat anti-mouse IgG-HRP (ThermoFisher), was diluted 1:1000 in assay buffer and 50 μL of this solution was added to each well. After a two hour incubation period, wells were washed four times with 250 μL PBS-T.
h. Next 50 μL of substrate, which was prepared by diluting 5 mg of OPD into 10 mL 1× Stable Peroxidase Substrate Buffer, was added to each well. The substrate was incubated for 15 minutes and the reaction was stopped by the addition of 50 μL of 1 M HCl to each well.
i. The absorbance of each well was determined using the Flurostar Omega (BMG Labtech) Spectrophotometer using an absorbance of 492 nm and a reference wavelength subtraction of 650 nm. The standard curve fitting was performed using GraphPad Prism© software with a 4-parameter curve fit with no weighting using a minimum of 6 points on the standard curve (Findlay and Dillard 2007).

Total IgE Detection ELISA

Reagents & Buffers

Polyclonal Rabbit anti-human IgE (Dako, A0094)

Peroxidase-conjugated goat anti-human IgE (Sigma, A9667)

IgE standard (WHO 75/502) (stock concentration 1 mg/ml, stored at −20° C.)

TMB ‘Substrate Reagent Pack’ (R&D, DY999, 4° C.)

Carbonate Buffer, pH 9.2 (4 ml 0.2M sodium carbonate (2.2 g/100 ml)+46 ml sodium bicarbonate (1.68 g/100 ml), to 200 ml with H₂O)

1% BSA/PBS

Wash buffer (0.05% Tween 20/PBS)

Protocol
1) Coating Plates
   a. Dilute anti-human IgE coating antibody 1:7000 in carbonate buffer
   b. OPTIONAL: dilute antigens to 5 μg/ml in carbonate buffer
   c. Add 100 μl/well diluted coating antibody (and antigens if applicable)
   d. Seal plate and incubate at 4° C. overnight
2) Wash and Block wells
   a. Flick out coating antibody
   b. Add 200 μL wash buffer per well
   c. Flick out and blot on tissue paper to remove excess wash buffer
   d. Repeat b and c an additional four times
   e. Add 100 μl/well 1% BSA/PBS
   f. Cover plate with lid and incubate for 1 hour at room temperature
3) Wash and add Supernatants and Standards
   a. Wash plate five times as described in step 2b-2d
   b. Dilute standard to 800 ng/ml (15 μl stock+210 μl 1% BSA/PBS)
   c. Add 50 μl 1% BSA/PBS to wells 2-12 of the standard row/s
   d. Add 50 μl of standard to the wells 1 and 2 of the standard row/s
   e. Mix well and transfer 50 μl sequentially to create a two-fold dilution (leaving the final well as a blank)
   f. Add 50 μl/well of samples (including +ve and −ve controls) in duplicate
   g. Seal plate and incubate overnight at 4° C. or for 2 hours at room temperature on a shaking platform
4) Wash and add detector
   a. Wash plate five times as described in step 2b-2d
   b. Dilute peroxidase-conjugated detection antibody to 1:500 in 1% BSA/PBS
   c. Add 100 μl/well
   d. Seal plate and incubate at room temperature for 1 hour on a shaking platform
5) Substrate Solution
   a. Wash plate five times as described in step 2b-2d
   b. Mix an equal volume of colour reagent A with colour reagent B
   c. Add 50 μl/well
   d. Incubate in dark for around 5-10 minutes
   e. Stop the reaction with 50 μl/well 3M sulphuric acid
6) Reading plates
   a. Read plate immediately after development
   b. Reference filter 450 nm

Results and Discussion

The capability for IgE alone to be taken up by cells and undergo lysosomal degradation, or to be recycled via the FcRn or potentially equivalent recycling and recovery pathways was assessed in an assay modified from that published by Grevy's et al 2018.

Grevys A, Nilsen J, Sand K M K, Daba M B, Øynebråten I, Bern M, McAdam M B, Foss S, Schlothauer T, Michaelsen T E, Christianson G J, Roopenian D C, Blumberg R S, Sandlie I, Andersen J T. A human endothelial cell-based recycling assay for screening of FcRn targeted molecules. Nat Commun. 2018 Feb. 12; 9(1):621

TABLE 1
Assessment of the recycling potential of IgE alone

| | 1 nM IgE | % IgE |
|---|---|---|
| IgE | Extracellular supernatant (remaining) | 99.00% |
| only | Uptake | 1.00% |
| | Extracellular supernatant (remaining prior to buffer change) | 99.80% |
| | Recycling | 0.00% |
| | Intracellular retention | 0.00% |
| | Undetected (degraded) | 0.20% |

Table shows percentage of IgE in each location

TABLE 2
Assessment of the recycling potential of biologic anti-IgE to capture IgE
and its capacity to internalise IgE and the efficiency of biologic recycling

| Biologic concentration: | | | | | | | | |
|---|---|---|---|---|---|---|---|---|
| 0.01 nM | 0.05 nM | 0.5 nM | 1 nM | 5 nM | 50 nM | 500 nM | 1000 nM | 2000 nM |

Biologic alone

| | | | | | | | | | | |
|---|---|---|---|---|---|---|---|---|---|---|
| Biologic | Extracellular | 5.45% | 2.00% | 1.65% | 3% | 6.00% | 18% | 26% | 28% | 31% |
| only | supernatant | | | | | | | | | |
| | (remaining) | | | | | | | | | |
| | Uptake | 93.50% | 97.00% | 95.00% | 95.55% | 93.45% | 82% | 74% | 72% | 69% |
| | Extracellular | 4.50% | 1.50% | 1.00% | 2.50% | 5.00% | 12.00% | 15.00% | 25.00% | 28.50% |
| | supernatant | | | | | | | | | |
| | (remaining | | | | | | | | | |
| | prior to buffer | | | | | | | | | |
| | change) | | | | | | | | | |
| | Recycling | 94.00% | 97.00% | 98.00% | 97.50% | 92.50% | 85.00% | 85% | 75% | 71% |
| | Intracellular | 1.00% | 1.00% | 0.50% | 0.00% | 1.50% | 1.50% | 0.00% | 0% | 0.5% |
| | retention | | | | | | | | | |
| | Undetected | 0.50% | 0.50% | 0.50% | 0% | 1.00% | 1.50% | 0% | 0.00% | 0.00% |
| | (degraded) | | | | | | | | | |

Table shows the percentage of biologic remaining in each location
Biologic + 1 nM IgE

| | | | | | | | | | | |
|---|---|---|---|---|---|---|---|---|---|---|
| Biologic + | Extracellular | 8% | 5.00% | 1.00% | — | — | — | — | — | — |
| 1 nM IgE | supernatant | | | | | | | | | |
| | (IgE remaining) | | | | | | | | | |
| | IgE Uptake | 92% | 95.00% | 99.00% | 100% | 100% | 100% | 100% | 100% | 100% |
| | Extracellular | 7.00% | 4.50% | 1.00% | — | — | — | — | — | — |
| | supernatant | | | | | | | | | |
| | (IgE remaining | | | | | | | | | |
| | prior to buffer | | | | | | | | | |
| | change) | | | | | | | | | |
| | IgE Recycling | — | — | — | — | — | — | — | — | — |
| | IgE | 92% | 94.00% | 98.00% | 95.00% | 93.00% | 90.00% | 79% | 65% | 52% |
| | Intracellular | | | | | | | | | |
| | retention | | | | | | | | | |
| | IgE Undetected | 1.00% | 1.50% | 1.00% | 5.00% | 7.00% | 10.00% | 21.00% | 35% | 48% |
| | (degraded) | | | | | | | | | |

Table shows the percentage of IgE in each location

TABLE 3
Assessment of the recycling potential of Omalizumab anti-IgE to capture IgE
and its capacity to internalise IgE and the efficiency of antibody recycling

| Omalizumab concentration: | | | | | | | | |
|---|---|---|---|---|---|---|---|---|
| 0.01 nM | 0.05 nM | 0.5 nM | 1 nM | 5 nM | 50 nM | 500 nM | 1000 nM | 2000 nM |

Omalizumab Only

| | | | | | | | | | | |
|---|---|---|---|---|---|---|---|---|---|---|
| Omalizumab | Extracellular | 0.50% | 0.80% | 1.50% | 1.15% | 2.00% | 7.55% | 15.25% | 25.00% | 36.15% |
| only | supernatant | | | | | | | | | |
| | (remaining) | | | | | | | | | |
| | Uptake | 99.50% | 98.00% | 96.00% | 97.50% | 96.55% | 92.00% | 81.45% | 72.00% | 62.50% |
| | Extracellular | 0.50% | 0.60% | 0.55% | 1.50% | 1.55% | 5.00% | 12.45% | 18.50% | 28.00% |
| | supernatant | | | | | | | | | |
| | (remaining | | | | | | | | | |
| | prior to buffer | | | | | | | | | |
| | change) | | | | | | | | | |
| | Recycling | 80.00% | 81.00% | 80.00% | 82.00% | 84.00% | 74.00% | 65.00% | 52.45% | 50.50% |
| | Intracellular | 19.50% | 18.40% | 19.45% | 15.50% | 13.45% | 14.00% | 7.55% | 12.00% | 1.5% |
| | retention | | | | | | | | | |
| | Undetected | 0.00% | 0.00% | 0.00% | 1.00% | 1.00% | 7.00% | 15.00% | 17.00% | 20.00% |
| | (degraded) | | | | | | | | | |

Table shows the percentage Omalizumab in each location
Omalizumab + 1 nM IgE

| | | | | | | | | | | |
|---|---|---|---|---|---|---|---|---|---|---|
| Omalizumab + | Extracellular | — | — | — | — | — | — | — | — | — |
| 1 nM IgE | supernatant | | | | | | | | | |
| | (IgE remaining) | | | | | | | | | |
| | IgE Uptake | 100% | 100% | 100% | 100% | 100% | 100% | 100% | 100% | 100% |
| | Extracellular | — | — | — | — | — | — | — | — | — |
| | supernatant | | | | | | | | | |
| | (IgE remaining | | | | | | | | | |
| | prior to buffer | | | | | | | | | |
| | change) | | | | | | | | | |
| | IgE Recycling | 42.00% | 45.00% | 48.00% | 50.55% | 51.00% | 53.00% | 55.00% | 50.65% | 51.15% |
| | IgE | 23.50% | 27.55% | 30.15% | 29.55% | 30.10% | 29.55% | 28.95% | 29.95% | 30.15% |
| | Intracellular | | | | | | | | | |
| | retention | | | | | | | | | |
| | IgE Undetected | 34.50% | 27.45% | 21.85% | 19.90% | 18.90% | 17.45% | 16.05% | 19.40% | 18.70% |
| | (degraded) | | | | | | | | | |

Table shows the percentage of IgE in each location

The data described in Table 1 demonstrate that in the absence of biologic-anti-IgE (example 1), IgE remains in the supernatant of HEK293-mFcRn cell culture with very little cellular uptake after 4 hours incubation with the cells prior to washing the cells. Following washing, there was no evidence of IgE being recycled, or being retained within the cells.

Assessment of Biologic Anti-IgE Effect on IgE Uptake, Cellular Retention and Recycling

Table 2 shows that increasing concentrations of the biologic anti-IgE alone, without IgE, were assessed in the HEK293-mFcRn/β2m recycling assay. The construct demonstrated rapid uptake by the FcRn endocytic transport mechanism such that at biologic concentrations between 0.01-5.00 nM>93% of the biologic was taken up from the medium by the transfected HEK293 cells within the 4-hour incubation period. In the presence of 1 nM IgE, there was complete removal of IgE from the cell culture medium within the 4 hour incubation window when in presence of the biologic anti-IgE between 1-2000 nM. At concentrations below 1 nM, when IgE was in excess of biologic anti-IgE, there was 8%, 5% and 1% of IgE remaining when incubated for 4 hours with 0.01 nM, 0.05 nM and 0.5 nM biologic anti-IgE respectively (Table 2).

Of the IgE taken up, the majority of IgE was retained within the cell with no IgE found in the recycled fraction after 4 hours. The undetected fraction of IgE, not recovered in either the cell incubation medium, nor in the cell lysate, is believed to be degraded.

Assessment of Omalizumab Anti-IgE Effect on IgE Uptake, Cellular Retention and Recycling

The data in Table 3 demonstrates that omalizumab is efficiently taken up by HEK293-hFcRn/β2m transfected cells, such that little remains in the supernatant 4 hours post-addition. Following the buffer change and a further 4 hours incubation, between 50 to 84% of omalizumab was recovered in the cell medium, with the remainder being retained within the cell.

When incubated in the presence of 1 nM IgE plus increasing concentration of omalizumab, after 4 hours incubation no IgE could be detected in the extra-cellular supernatant.

Following the exchange of buffers and washing of the cells with buffer, warmed medium was added to the HEK293 cells as described in the materials and methods above. After 4 hours incubation, the extra-cellular supernatant was removed and the presence of IgE measured, whilst the HEK293-hFcRn/β2m cells were lysed and the intra-cellular quantity of IgE quantitated in a suitable ELISA assay. From the studies, it can be observed that between 42-55% of the IgE was recovered in the extra-cellular supernatant depending on the concentration of omalizumab tested. It is thought this may be a consequence of the stable IgE-omalizumab complex being recycled through the endosomal recycling pathway, which may account for the longevity of IgE-anti-IgE complexes observed in patients treated with omalizumab. Of the remaining IgE, between 23-30% could be measured in the cell lysate, with the remainder (between 16-34% undetected, potentially degraded) (Table 3).

The studies demonstrate the biologic anti-IgE to be a more effective agent for removal of IgE than omalizumab. Whilst biologic anti-IgE efficiently bound IgE and permitted cellular uptake by HEK293-hFcRn cells, there was no detectable IgE in the extra-cellular supernatant following the washing and incubation protocol, suggesting that the IgE did not leave the cell, as confirmed by cell lysis and measurement of intra-cellular IgE levels. By contrast, omalizumab is unable to efficiently release IgE within the endosome, so the IgE-omalizumab complex gets recycled back to the circulation with less than 50% of the IgE being retained within the cell when omalizumab is dosed in molar excess. These data suggest that the calcium sensitive binding mechanism inherent within biologic anti-IgE is a highly efficient mechanism to release the bound target (IgE), whilst still permitting recycling of the Biologic anti-IgE itself, as evidenced by the efficiency of biologic anti-IgE when dosed such that IgE was in vast molar excess (Table 2).

### Example 4: Evaluation of Biologic Anti-IgE Binding to IgE by Surface Plasmon Resonance Using the BIACore

Methods and Materials:

BIACore Studies: General Surface Plasmon Resonance Protocol

Immobilisation was performed by direct amine coupling to carboxymethylated sensor chip surface (CM5 chips, GE Healthcare). The carboxymethylated dextran surface of each CM5 chip was activated by a 420 second injection of 0.1 M N-hydroxysuccinimide (NHS) and 0.4 M 1-ethyl-3-(3-dimethylaminopropyl)-carbodiimide (EDC) at a 1:1 ratio in deionised water. The NHS/EDC solution reacts with free carboxyl-groups present on the chip and results in the generation of reactive succinimide esters that can react with surface exposed lysine residues of proteins, thus immobilising them on the surface. Proteins were injected over the NHS/EDC activated surface at a concentration of 10 μg/ml in 10 mM sodium acetate pH 5.0 in 60-300 second pulses, until the desired level of immobilisation was achieved. Any remaining active carboxymethylated groups were blocked by 1 M ethanolamine, pH 8.5, which was injected over the chip for 600 seconds. Reference cells were prepared using the same procedure, except that buffer was injected over the surface instead of protein. All immobilisations were performed at 25° C. with a flow rate of 20 μl/min.

BIACore Binding Study: derCD23 Binding to IgE-Fc

SPR experiments were performed to determine the effect of derCD23 on the interaction between IgE-Fc and α-Cε4 Fab, and steady-state analysis was employed to quantify these effects in terms of K_{D} and Bₘₐₓ. An α-Cε4 Fab CM5 sensor surface was prepared using amine-coupling and a mock amine-coupled surface was used as a reference-subtraction control. 80 nM IgE-Fc was then injected to generate an immobilised 1:1 α-Cε4 Fab/IgE-Fc complex. Following a short SPR buffer injection over the α-Cε4 Fab coupled surface to initiate a short dissociation phase, a two-fold serial titration of derCD23, from 4000 nM to 31 nM, was injected over the 1:1 α-Cε4 Fab/IgE-Fc complex. The derCD23 injection was then followed by a dissociation phase, and regeneration of the α-Cε4 Fab captured IgE-Fc surface (FIG. 8). Injections were performed at a flow rate of 25 μl·min⁻¹ in a running buffer of 10 mM HEPES, pH 7.4, 150 mM NaCl, 4 mM CaCl₂, and 0.005% (v/v) surfactant P-20 (GE Healthcare). All experiments were run in duplicate and gave highly reproducible results using a BIACore T200 instrument (GE Healthcare), with monophasic kinetic fitting giving rise to a K_{D} of 1.8×10⁻⁶ M.

BIACore Binding Study: Anti-IgE Biologic Construct Binding to IgE-Fc

The interaction of CD23 binding to α-Cε4 Fab-captured IgE-Fc, and the binding curves for the interactions between the three anti-IgE biologics and α-Cε4 Fab captured IgE-Fcs were assessed. The test articles in this experiment were biologic anti-IgE molecules, which comprise a pair of CD23 monomers, but in which the length of linker between the IgE binding component (the CD23 monomer) and the FcRn binding component (IgGFc) was varied between having no linker)(anti-IgE⁰), to having 3 (anti-IgE³), or 4 (anti-IgE⁴), repeats of the (G4S) linker sequence. A 1:1 α-Cε4 Fab/IgE-Fc complex was immobilised on a CM5 sensor surface. A two-fold serial titration of the anti-IgE molecules, from 4000 nM to 31 nM, was injected over the α-Cε4 Fab captured IgE-Fc, followed by a dissociation phase (FIG. 9a A-C), and regeneration of the surface. The change in SPR response was then used to measure the ability of the α-Cε4 Fab captured IgE-Fc to bind the anti-IgE molecules. Data fitting using biphasic kinetic models gave rise to two K_{D} values, K_{D1} 1-2×10⁻⁶ M and K_{D2} 1-4×10⁻⁸M, with the longer linkers producing the lower concentration K_{D} in each case.

BIACore Binding Study: Effect of Varying IgE-Fc Immobilisation Levels on Anti-IgE Molecule Binding Characteristics

Ligand density may affect to what extent an SPR experiment measures intrinsic or functional affinity. At high ligand densities, it is possible that a multivalent analyte may simultaneously bind two or more ligands. If the kinetics of the interaction sites are the same and independent, the first interaction will be dependent on the intrinsic affinity of the site. The association of the subsequent sites is favoured because of the high local concentration of analyte. In performing this set of experiments, sensor chip surfaces were prepared by covalently immobilising α-Cε4 Fab on the chip surface at a density that would ensure the formation of a 1:1 complex between IgE-Fc and α-Cε4 Fab. Three different concentrations of IgE-Fc (80 nM, 160 pM and 40 pM) were injected over the immobilised α-Cε4 Fab capturing molecule, giving rise to average molecular spacings of 40 nm, 80 nm and 110 nm, respectively. The average molecular spacing measurements were chosen based on the assumption that at lower immobilised levels of IgE-Fc, the anti-IgE molecules would behave less bivalently, and a monophasic interaction would be favoured. Varying concentrations of anti-IgE molecules, (4000 nM-31 nM) were injected over the α-Cε4 Fab/IgE-Fc surfaces.

The SPR response (resonance units) was used to measure the specific binding of anti-IgE biologics to α-Cε4 Fab captured IgE-Fc. Following each injection, there was an 800 s dissociation phase, and the α-Cε4 Fab captured IgE-Fc was then regenerated by three 60 s pulses of 10 mM glycine pH 2.5, and one pulse of 5 mM NaOH to regenerate the surface for the next cycle. Injections were performed at a flow rate of 25 μl·min⁻¹ in a running buffer of 10 mM HEPES, pH 7.4, 150 mM NaCl, 4 mM CaCl₂, and 0.005% (v/v) surfactant P-20. These experimental binding measurements were performed at 25° C. In all cases, standard double referencing data subtraction methods were used, and kinetic fits were performed using Origin software (OriginLab).

Results & Discussion

The data in FIG. 8 clearly demonstrates that CD23 monomer is able to bind IgE with relatively low affinity. The data in FIG. 9a demonstrates that CD23 monomers, arranged as pairs, are able to bind IgE with improved affinity compared to a single monomer, and that the introduction of a linker between CD23 monomeric component and the FcRn binding component shows improved binding. The plots shown in FIG. 9b show that for each of the anti-IgE molecules, larger separation between the immobilised IgE molecules leads to faster dissociation of the complexes that are formed by binding to IgE, and that increasing the linker length of the anti-IgE biologics, reduces this effect.

### Example 5: Evaluation of Anti-IgE Biologics with Varying Linker Length on Ability to Inhibit IgE-Mediated Basophil Degranulation

It is well established that IgE binding to the high affinity receptor FcεRI and the consequent cross-linking of bound IgE, in the presence of allergen, causes activation of effector cells such as mast cells and basophils, triggering the release of inflammatory mediators, including histamine to cause an allergic response. This study investigated the potential effect of the introduction of linkers to alter the spatial reach of IgE-binding CD23 monomers, organised as pairs, to bind IgE and prevent IgE-mediated activation and degranulation of basophilic effector cells.

Methods and Materials:

Basophil Degranulation Assay

The assay methods and materials for the basophil degranulation assays were as described in Example 2. The test articles in this Example were biologic anti-IgE molecules, which comprise a pair of CD23 monomers, but in which the length of linker between the IgE binding component (the CD23 monomer) and the FcRn binding component (IgGFc) was varied between having no linker (anti-IgE⁰), to having 3 (anti-IgE³), or 4 (anti-IgE⁴), repeats of the (G4S) linker sequence. This has the effect of extending the spatial reach of the IgE binding component.

Results & Discussion:

Each of the biologic anti-IgE's tested was able to inhibit IgE mediated degranulation by effectively blocking the interaction between IgE and the high affinity IgE receptor, FcεRI, expressed on the surface of RBL-SX38 human basophilic cell line. The potency and efficacy of each of the anti-IgE biologics differed. Biologic anti-IgE⁰, which comprises a pair of CD23 monomers but no linker between the IgE binding component (the CD23 monomer) and the FcRn binding component (IgGFc), was able to partially inhibit IgE mediated degranulation of basophils, but with only a maximal 50% efficacy. The introduction of a linker sequence between the CD23 monomer and the IgG-Fc markedly increased both efficacy and potency, reaching ˜90% efficacy when the linker comprised 3 repeats of the G4S linker, and reaching 100% efficacy, when the linker length was increased to a (G4S)₄ repeat. Accordingly, the observed IC₅₀'s demonstrated increased potency with increasing linker length, decreasing from >300 nM for anti-IgE⁰, to between 10-30 nM on the addition of linkers.

1. A protein construct comprising:
a) at least two monomers each of which comprises a C-type lectin domain of CD23, wherein each monomer can bind to IgE; and
b) an entity which can bind to the neonatal Fc receptor (FcRn);
wherein said protein construct comprises a linker, and wherein said linker is used to link said monomer comprising a C-type lectin domain of CD23 to said entity which can bind to FcRn,
wherein said C-type lectin domain of CD23 corresponds to S156 to S321 of SEQ ID NO:1 (SEQ ID NO:9), or E133 to S321 of SEQ ID NO:1 (SEQ ID NO:12), or a fragment thereof, or an equivalent sequence in a non-human species of CD23, or a sequence with at least 80% identity thereto.

2. The protein construct of claim 1, wherein said construct contains two monomers, or more than two monomers, preferably 4 or 6 monomers.

3. The protein construct of claim 1 or claim 2, wherein said C-type lectin domain of CD23 comprises or corresponds to V159-P290 of SEQ ID NO:1 (SEQ ID NO:6) or C160-C288 of SEQ ID NO:1 (SEQ ID NO:7) or F170-L277 of SEQ ID NO:1 (SEQ ID NO:8), or an equivalent sequence in a non-human species of CD23, or a sequence with at least 80% identity thereto.

4. The protein construct of any one of claims 1 to 3, wherein said C-type lectin domain of CD23 comprises or corresponds to S156 to A292 of SEQ ID NO:1 (SEQ ID NO:15), preferably E133 to A292 of SEQ ID NO:1 (SEQ ID NO:10), or comprises or corresponds to S156 to C288 of SEQ ID NO:1 (SEQ ID NO:31), or a fragment thereof, or an equivalent sequence in a non-human species of CD23, or a sequence with at least 80% identity thereto.

5. The protein construct of any one of claims 1 to 4, wherein said C-type lectin domain of CD23 comprises or corresponds to S156 to E298 of SEQ ID NO:1 (SEQ ID NO:13), preferably E133 to E298 of SEQ ID NO:1 (SEQ ID NO:11), or a fragment thereof, or an equivalent sequence in a non-human species of CD23, or a sequence with at least 80% identity thereto.

6. The protein construct of any one of claims 1 to 5, wherein each monomer binds to IgE with an affinity of 0.1-3 μM.

7. The protein construct of any one of claims 1 to 6, wherein said entity which can bind to FcRn comprises an Fc region, preferably an IgG-Fc region, or a fragment or variant thereof, or albumin or a fragment or variant thereof, or a binding protein for an IgG antibody or albumin, or a binding protein for FcRn.

8. The protein construct of claim 7, wherein said entity which can bind to FcRn comprises an IgG1-Fc region or a fragment or variant thereof, or human serum albumin or a fragment or variant thereof, or a binding protein for an IgG1 antibody or human serum albumin.

9. The protein construct of claim 7 or claim 8, wherein said binding protein comprises an antibody or antibody fragment, preferably a sdAb, or comprises a non-immunoglobulin based single domain binding protein, preferably a fibronectin or fibronectin-based molecule, an affimer, an ankyrin repeat protein, a lipocalin, a human A-domain, a staphylococcal Protein A, a thioredoxin, a gamma-B-crystallin, or a ubiquitin based molecule.

10. The protein construct of any one of claims 1 to 9, wherein said linker is a peptide linker.

11. The protein construct of any one of claims 1 to 10, wherein said binding of each monomer of part a) of the construct to IgE and/or said binding of part b) of the construct to FcRn is sensitive to endosomal conditions.

12. The protein construct of claim 11, wherein said binding of part a) of the construct to IgE is reduced at pH 6.0 or 6.5 compared to pH 7.4, or is reduced at endosomal calcium levels compared to serum calcium levels.

13. The protein construct of claim 11 or claim 12, wherein said binding of part b) of the construct to FcRn is increased at pH 6.0 or 6.5 compared to pH 7.4, or is increased at endosomal calcium levels compared to serum calcium levels.

14. The protein construct of any one of claims 1 to 13, wherein the at least two monomers result in increased avidity of binding to IgE compared to the sum of binding affinities of the individual monomers.

15. One or more nucleic acid molecules comprising nucleotide sequences that encode the protein construct of any one of claims 1 to 14; or
one or more expression vectors comprising such nucleic acid molecules; or one or more host cells comprising said expression vectors, nucleic acid molecules or protein constructs of any one of claims 1 to 14.

16. A method of producing the protein construct of any one of claims 1 to 14, said method comprising the steps of (i) culturing a host cell comprising one or more of the expression vectors or one or more of the nucleic acid sequences as defined in claim 15 under conditions suitable for the expression of the encoded protein construct; and optionally (ii) isolating or obtaining the expressed protein construct from the host cell or from the growth medium/supernatant.

17. A method of producing the protein construct of any one of claims 1 to 14, said method comprising the steps of (i) contacting an affinity matrix to which IgE Fc has been immobilised with a construct of any one of claims 1 to 14 under conditions such that said construct binds to the IgE Fc on the affinity matrix; and (ii) eluting the construct from the affinity matrix under conditions such that the construct no longer binds to the IgE Fc on the affinity matrix.

18. The method of claim 17, wherein in step (i) such conditions are those corresponding to serum calcium or pH levels, preferably calcium levels of 1 to 2 mM, or a pH of at or about pH 7.4; and/or in step (ii) such conditions are those corresponding to endosomal calcium or pH levels, preferably calcium levels of 3-30 μM or a pH of at or about pH 5.0 to 6.5.

19. A composition, preferably a pharmaceutically acceptable composition, comprising a protein construct of any one of claims 1 to 14, or one or more nucleic acid molecules or expression vectors of claim 15.

20. The protein construct of any one of claims 1 to 14 or the one or more nucleic acid molecules or expression vectors of claim 15 for use in therapy, preferably for use in anti-IgE therapy or for use in the treatment or prevention of an IgE related disease or condition.

21. Use of the protein construct of any one of claims 1 to 14 or the one or more nucleic acid molecules or expression vectors of claim 15, in the manufacture of a medicament or composition for use in anti-IgE therapy or in the treatment or prevention of an IgE related disease or condition.

22. A method of treatment or prevention of an IgE related disease or condition, wherein said method comprises the step of administering to a patient in need thereof a therapeutically effective amount of the protein construct of any one of claims 1 to 14 or the one or more nucleic acid molecules or expression vectors of claim 15.

23. (canceled)


SEQUENCE LISTING (38 sequences)

SEQ ID NO: 1 - protein, 321 aa, Human
MEEGQYSEIEELPRRRCCRRGTQIVLLGLVTAALWAGLLTLLLLWHWDTTQSLKQLEERAARNVSQVSKNLESHHGDQMAQKSQSTQISQELEELRAEQQRLKSQDLELSWNLNGLQADLSSFKSQELNERNEASDLLERLREEVTKLRMELQVSSGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAESMGPDSRPDPDGRLPTPSAPLHS

SEQ ID NO: 2 - protein, 320 aa, Human
MNPPSQEIEELPRRRCCRRGTQIVLLGLVTAALWAGLLTLLLLWHWDTTQSLKQLEERAARNVSQVSKNLESHHGDQMAQKSQSTQISQELEELRAEQQRLKSQDLELSWNLNGLQADLSSFKSQELNERNEASDLLERLREEVTKLRMELQVSSGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAESMGPDSRPDPDGRLPTPSAPLHS

SEQ ID NO: 3 - protein, 274 aa, Human
DTTQSLKQLEERAARNVSQVSKNLESHHGDQMAQKSQSTQISQELEELRAEQQRLKSQDLELSWNLNGLQADLSSFKSQELNERNEASDLLERLREEVTKLRMELQVSSGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAESMGPDSRPDPDGRLPTPSAPLHS

SEQ ID NO: 4 - protein, 241 aa, Human
QKSQSTQISQELEELRAEQQRLKSQDLELSWNLNGLQADLSSFKSQELNERNEASDLLERLREEVTKLRMELQVSSGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAESMGPDSRPDPDGRLPTPSAPLHS

SEQ ID NO: 5 - protein, 220 aa, Human
LKSQDLELSWNLNGLQADLSSFKSQELNERNEASDLLERLREEVTKLRMELQVSSGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAESMGPDSRPDPDGRLPTPSAPLHS

SEQ ID NO: 6 - protein, 132 aa, Human
VCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTP

SEQ ID NO: 7 - protein, 129 aa, Human
CNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATC

SEQ ID NO: 8 - protein, 108 aa, Human
FQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKL

SEQ ID NO: 9 - protein, 166 aa, Human
SGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAESMGPDSRPDPDGRLPTPSAPLHS

SEQ ID NO: 10 - protein, 160 aa, Human
EASDLLERLREEVTKLRMELQVSSGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPA

SEQ ID NO: 11 - protein, 166 aa, Human
EASDLLERLREEVTKLRMELQVSSGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAE

SEQ ID NO: 12 - protein, 189 aa, Human
EASDLLERLREEVTKLRMELQVSSGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAESMGPDSRPDPDGRLPTPSAPLHS

SEQ ID NO: 13 - protein, 143 aa, Human
SGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAE

SEQ ID NO: 14 - protein, 96 aa, Human
WVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGA

SEQ ID NO: 15 - protein, 137 aa, Human
SGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPA

SEQ ID NO: 16 - protein, 5 aa, Artificial sequence
  Linker (base unit)
GGGGS

SEQ ID NO: 17 - protein, 15 aa, Artificial sequence
  Linker (x3 repeats)
GGGGSGGGGSGGGGS

SEQ ID NO: 18 - protein, 30 aa, Artificial sequence
  Linker (x6 repeats)
GGGGSGGGGSGGGGSGGGGSGGGGSGGGGS

SEQ ID NO: 19 - protein, 445 aa, Artificial sequence
  whole secreted sequence of hCD23-G4S3-mIgG2a Fc (signal peptide removed)
EASDLLERLREEVTKLRMELQVSSGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAESMGPDSRPDPDGRLPTPSAPLHSGGGGSGGGGSGGGGSASISAMVRSPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 20 - protein, 23 aa, Mouse
MSVPTQVLGLLLLWLTDARCDGA

SEQ ID NO: 21 - protein, 468 aa, Artificial sequence
  whole ORF sequence of hCD23-G4S3-mIgG2a Fc (including secretory signal peptide)
MSVPTQVLGLLLLWLTDARCDGAEASDLLERLREEVTKLRMELQVSSGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAESMGPDSRPDPDGRLPTPSAPLHSGGGGSGGGGSGGGGSASISAMVRSPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 22 - protein, 23 aa, Human
EASDLLERLREEVTKLRMELQVS

SEQ ID NO: 23 - protein, 137 aa, Human; identical to SEQ ID NO: 15

SEQ ID NO: 24 - protein, 6 aa, Human
SEGSAE

SEQ ID NO: 25 - protein, 23 aa, Human
SMGPDSRPDPDGRLPTPSAPLHS

SEQ ID NO: 26 - protein, 241 aa, Artificial sequence
  mutant mouse IgG2a Fc
ASISAMVRSPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 27 - protein, 330 aa, Human [sequence omitted]

SEQ ID NO: 28 - protein, 326 aa, Human
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 29 - protein, 377 aa, Human
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 30 - protein, 5 aa, Human
EGSAE

SEQ ID NO: 31 - protein, 133 aa, Human
SGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATC

SEQ ID NO: 32 - protein, 133 aa, Canine
NGSECNTCPEKWLNFQRKCYYFGEEPKKWIQARFACSKLQGRLASIHSQEEQDFLARYANKKGTWIGLRDLDREGEFIWMDENPLNYSNWRPGEPNNGGQGEDCVMMQGSGQWNDAFCGSSLDGWVCDRLATC

SEQ ID NO: 33 - protein, 245 aa, Canine
KTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 34 - DNA, 1407 bp, Artificial sequence
  Artificial sequence
atgagtgtgcccactcaggtcctggggttgctgctgctgtggcttacagatgccagatgtgatggcgccgaagcttccgacctgctggaacggctgcgggaggaagtgaccaagctgcggatggaactgcaggtgtccagcggcttcgtgtgcaacacctgccccgagaagtggatcaacttccagcggaagtgctactacttcggcaagggcaccaagcagtgggtgcacgccagatacgcctgcgacgacatggaaggccagctggtgtccatccacagccccgaggaacaggacttcctgaccaagcacgccagccacaccggcagctggatcggcctgcggaacctggacctgaagggcgagttcatctgggtggacggcagccacgtggactacagcaactgggcccctggcgagcccacctccagaagccagggcgaggactgcgtgatgatgcggggcagcggccggtggaacgacgccttctgcgaccggaagctgggcgcctgggtgtgcgaccggctggccacctgcaccccccctgccagcgagggcagcgccgagagcatgggccccgacagcaggcccgaccccgacggcagactgcccacccccagcgcccctctgcacagcggcggcggcggcagcggcggcggcggcagcggcggcggcggcagcgccagcatatcggccatggttagatctcccagagggcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggcgttcgcatgcgcggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 35 - DNA, 26 bp, Artificial sequence
  primer
gtctgtgtgtgatcagtgtgaggctg

SEQ ID NO: 36 - DNA, 46 bp, Artificial sequence
  primer
taagataaacctgcctccctccctcccagggctccatccagctgtg

SEQ ID NO: 37 - DNA, 32 bp, Artificial sequence
  primer
tgatcacacacagacatgagtgtgcccactca

SEQ ID NO: 38 - DNA, 44 bp, Artificial sequence
  primer
gagggaggcaggtttatcttatcatttacccggagtccgggaga